(19) United States
(12) Patent Application Publication
(10) Pub. No.: US 2021/0389297 A1
(43) Pub. Date: Dec. 16, 2021

(54) New Apparatus and Methods for Disease Detection and Treatment

(71) Applicant: AnPac Bio-Medical Science Co., Ltd., Tortola (VG)

(72) Inventors: Chris C. Yu, Conneautville, PA (US); Xuedong Du, Shanghai (CN)

(73) Assignee: AnPac Bio-Medical Science Co., Ltd., Tortola (VG)

(21) Appl. No.: 17/282,770

(22) PCT Filed: Oct. 7, 2019
(86) PCT No.: PCT/US2019/055060

§ 371 Date: Apr. 4, 2021

Related U.S. Application Data
(63) Continuation of application No. PCT/US2019/028785, filed on Apr. 23, 2019.
(60) Provisional application No. 62/865,816, filed on Jun. 24, 2019.
(60) Provisional application No. 62/830,354, filed on Apr. 5, 2019.
(60) Provisional application No. 62/818,909, filed on Mar. 15, 2019.
(60) Provisional application No. 62/776,605, filed on Dec. 7, 2018.
(60) Provisional application No. 62/741,843, filed on Oct. 5, 2018.

(51) Int. Cl. G01N 33/487; G01N 27/02

(52) U.S. Cl. CPC G01N 33/48728; G01N 27/02

(57) ABSTRACT

The invention relates to apparatus and methods for apparatus for simultaneously detecting presence or monitoring progression of two or more types of cancer in a biological subject, comprising: a channel or chamber in which the biological subject passes through; and at least one detector placed partially or completely alongside one or more side walls of the channel or chamber, wherein the at least one detector is configured to detect at least one bio-physical or physical property of the biological subject. The invention also relates to apparatus and methods for apparatus for treating the detected diseases or cancers.

### CROSS-REFERENCE TO RELATED APPLICATIONS

This application claims priority to U.S. Application No. 62/741,843, filed Oct. 5, 2018, U.S. Application No. 62/776,605, filed Dec. 7, 2018, U.S. Application No. 62/818,909, filed Mar. 15, 2019, U.S. Application No. 62/830,354, filed Apr. 5, 2019, PCT Application No. PCT/US2019/028785, filed Apr. 23, 2019, and U.S. Application No. 62/865,816, filed Jun. 24, 2019, the contents of all of which are incorporated herein by reference in their entirety.

### BACKGROUND OF THE INVENTION

Many diseases are difficult to be detected by a single approach or methodology. In particular, many serious diseases with high morbidity and mortality, including cancer and heart diseases, are difficult to diagnose at an early stage with high sensitively, specificity and efficiency, by using one piece of detection equipment. Current disease diagnosis devices typically detect and rely on a single type of macroscopic data and information such as body temperature, blood pressure, or scanned images of the body. For example, to detect serious diseases such as cancer, each of the diagnosis apparatus commonly used today is based on one imaging technology, such as x-ray, CT scan, or nuclear magnetic resonance (NMR). While used in combination, these diagnosis apparatuses provide various degrees of usefulness in disease diagnosis. However, each of them alone cannot provide accurate, conclusive, efficient, and cost-effective diagnosis of such serious diseases as cancer at an early stage, and is difficult to detect multiple types of cancer at the same time. Further, many of the existing diagnosis apparatus have a large size and are invasive with large footprint, such as x-ray, CT scan, or nuclear magnetic resonance (NMR).

Even the newly emerged technologies such as those deployed in DNA tests usually rely on a single diagnosis technology and cannot provide a comprehensive, reliable, accurate, conclusive, and cost-effective detection for a serious disease. In recent years, there have been some efforts in using nano technologies for various biological applications, with most of the work focused on one type of gene mapping and moderate developments in the field of disease detection. For instance, Pantel et al. discussed the use of a MicroEelectroMechanical Systems (MEMS) sensor for detecting cancer cells in blood and bone marrow in vitro (see, e.g., Klaus Pantel et al., Nature Reviews, 2008, 8, 329); Kubena et al. disclose in U.S. Pat. No. 6,922,118 the deployment of MEMS for detecting biological agents; and Weissman et al. disclose in U.S. Pat. No. 6,330,885 utilizing MEMS sensor for detecting accretion of biological matter.

In sum, to date, most of above described technologies have been limited to isolated diagnosis technology for sensing, using systems of relatively simple constructions and large dimensions but often with limited functions, and lack sensitivities and specificities. Further, the existing technologies require multiple times detection by multiple apparatus. This will increase costs and affect achieved degree of sensitivity and specificity as well.

Current cancer screening and prognosis IVD methods typically include bio-markers, circulating tumor cells (CTC), and genomics (such as circuiting tumor-DNA (ct-DNA)). While each of the above-mentioned technology offer a number of advantages, they also have a number of limitations, which include inability to simultaneously detect multiple types of cancer, inability to detect cancer early, relatively low sensitivity and specificity, and in some cases, inability to detect certain types of cancer (for example, esophageal cancer and brain tumor). Bio-markers are not effective for early stage cancer detection, but are limited for cancer types.

In the case of CTC and ct-DNA, signals occur only after solid tumor has been formed, making early stage cancer detection relatively. See, e.g., Ji et al., J Clin Oncol 33, 2015; Xuedong Du et al., J Clin Oncol 33, 2015; Jiang et al., J Clin Oncol 33, 2015; Tao et al., J Clin Oncol 33, 2015; Bettegowda et al., Science Translational Med., 2014, 6 (224):224; Phallen et al., Science Translational Med., 2017, 9 (403): 2415; Khoo et al., Science Advances, 2016, 2 (7):e1600274; Garcia-Murillas et al., Sci. Translational Med., 2015, 7 (302): 302; Abbosh et al., Nature, 2017, 545 (7655):446-451; and Herbst et al., Nature, 2018, 553 (7689):446.

To date, conventional cancer screening, detection and/or treatment methods lack markers or signals to effectively target or impact multiple types of cancer at the same time (e.g., in one test or treatment).

These drawbacks call for novel solutions that provide reliable and flexible diagnosis apparatus using multiple diverse technologies and bring improved accuracy, sensitivity, specificity, efficiency, non-invasiveness, practicality, conclusive, and speed in early-stage disease detection, particularly in screening or detection of multiple cancer types, at reduced costs.

### SUMMARY OF THE INVENTION

The present invention in general relates to a novel technology for detecting disease, in which a number of different classifications of biological information are collected in a device and processed or analyzed.

It also relates to a novel technology for assessing risk levels of disease and cancer occurrence, and differentiating healthy individuals from possible disease or cancer individuals.

In traditional technology, typically only one level of biological information is collected (one dimensional), while in this novel technology, at least two levels (classifications) of information can be collected (seven dimensional, or seven factor interactions). Compared with traditional technology which typically focuses on one parameter or one level (for example, bio-marker at protein level), signal and information collected in this novel technology can be collected in a number of forms, and non-linearly amplified. There are additional 2-factor and three-factor interactions which can be collected and analyzed, which maybe missing in other technologies, since they typically only measure one type of biological information.

Existing cancer screening tests and treating methods lack the ability to effectively detect and/or impact multiple types of cancer at the same time (e.g., in one test or treatment). Compared with those traditional technologies which typically focus on one type of disease in one test, a significantly increased number of diseases - particularly cancers or pre-cancer diseases (e.g., over 20 types of cancer)—can be simultaneously detected, or even treated, by this novel technology.

Specifically, this novel technology can be used for cancer screening, assisting in diagnosis, prognosis, and follow-up tests with improved sensitivity and specificity, ability to detect cancer early, ability to detect major diseases, pre-cancer diseases and over 20 types of cancer, cost effective, and no side effects. Particularly, the present invention provides novel apparatus and methods for simultaneously detecting and/or treating the presence or monitoring the progression of multiple types of diseases, including cancer.

The novel technology offers several advantages that cannot be achieved by the traditional technology: (1) ability proven so far to detect as many as over 20 cancer types in one test, including some cancer types which cannot be detected by other in vitro tests (e.g., esophageal cancer, cerebral cancer), covering over 80% of all cancer incidences; (2) capability of early stage cancer detection; (3) high sensitivity and specificity (75%˜90% on over 20 types of cancer); (4) no side effects; (5) high speed, fully automated operations without human intervention; (6) statistical difference between cancer group and non-cancer disease group including inflammation—significantly lower false positives (higher specificity); (7) easy process, no difference between fasting blood testing and non-fasting blood testing, and (8) highly cost effective.

In addition, compared to conventional technologies, the novel cancer treatment technology of this invention has a number of major, unexpected advantages—including, e.g., lower costs, far less side efforts, easier recovery, capability of cancer prevention, improved survival rate, and ease of use and likely widespread clinical uses. Notably, the novel cancer treatment of this invention can use a low dosage and/or a weak field and/or energy for treatment.

Accordingly, one aspect of this invention relates to an apparatus for simultaneously detecting presence or monitoring progression of two or more types of cancer in a biological subject, comprising: a channel or chamber in which the biological subject passes through; at least one detector placed partially or completely alongside one or more side walls of the channel or chamber, wherein the at least one detector is configured to detect at least one bio-physical or physical property of the biological subject, and the detected bio-physical or physical property is collected for analysis to determine whether each of the two or more types of cancer is likely to be present with the biological subject or to determine the status of each cancer, thereby providing the ability to simultaneously determine or monitor progression of the two or more types of cancer.

In some embodiments, the bio-physical property comprises a mechanical property, an acoustical property, an optical property, an electrical property, an electro-magnetic property, or an electro-mechanical property.

In some further embodiments, electronic property can be surface charge, surface potential, resting potential, electrical current, electrical field distribution, surface charge distribution, cell electronic properties, cell surface electronic properties, dynamic changes in electronic properties, dynamic changes in cell electronic properties, dynamic changes in cell surface electronic properties, dynamic changes in surface electronic properties, electronic properties of cell membranes, dynamic changes in electronic properties of membrane surface, dynamic changes in electronic properties of cell membranes, membrane ion channel properties, resting membrane potential, trans-membrane potential, depolarized trans-membrane potential, electrical dipole, electrical quadruple, oscillation in electrical signal, electrical current, capacitance, three-dimensional electrical or charge cloud distribution, electrical properties at telomere of DNA and chromosome, DNA surface charge, DNA surrounding media electrical properties, quantum mechanical effects, capacitance, or impedance. For example, the bio-physical property may be quantum mechanical effects that affect gene replications and mutations.

In some embodiments, the bio-physical property comprises a trans-membrane potential, a membrane voltage, a membrane potential, membrane ion channel properties, resting membrane potential, trans-membrane potential, depolarized trans-membrane potential, a zeta potential, an impedance, an optical reflective index, an optical refractive index, potassium ions, sodium ions, chloride ions, nitride ions, calcium ions, an electro-static force, an electro-static force acting on cells, an electro-static force acting on DNA double helix, an electro-static force acting on RNA, an electrical charge on cell membrane, an electrical charge on DNA double helix, an electrical charge on RNA, quantum effects, near-field electrical properties, near-field electro-magnetic properties, membrane bilayer properties, ion types and/or concentrations, ion permeability, electrical current, electrical conductance, capacitance, or electrical resistance.

In some embodiments, the apparatus detects one or more bio-physical or physical properties each selected from the group consisting of electrolyte, potassium ion concentration, sodium ion concentration, chloride ion concentration, calcium ion concentration and distribution, net charge in extracellular region, ion concentration in extracellular, gravitational field, mechanical field, quantum mechanical field, magnetic field, electro-magnetic field, electrical field, electrical current, electrical resistance, electrical conductance, capacitance, membrane potential, membrane ion channel properties, resting membrane potential, trans-membrane potential, depolarized trans-membrane potential, magnetic field, electrical field, electro-magnetic field, and quantum field in the biological subject.

In some further embodiments, the apparatus directly or indirectly measures the quantum mechanical effects; and/or directly or indirectly measures ions or ion levels in a liquid sample of the biological subject; and/or directly or indirectly measures ion levels or concentrations by a bio-chemistry or electrode method; and/or directly or indirectly measures potassium ions; and/or directly or indirectly measures concentration of potassium ions; and/or directly or indirectly measures ion permeability.

In some embodiments, the apparatus directly or indirectly measures one or more ions selected from potassium ions, sodium ions, chloride ions, nitride ions and calcium ions; and/or directly or indirectly measures the concentration(s) of one or more ions selected from the group consisting of potassium ions, sodium ions, chloride ions, nitride ions and calcium ions.

In some embodiments, the biophysical physical property is related to and responsible for cell to cell interactions, cell signal, cell surface properties, cell electro-static force, cell repulsive force, DNA surface properties, DNA surface charge, DNA surrounding media electrical properties, quantum mechanical effects, gene mutation frequencies, or quantum mechanical effects.

In some embodiments, the biological subject is a liquid sample. For example, the biological subject may include a body fluid or tissue. More specifically, the body fluid may comprise whole blood, serum, plasma, sweat, tear, or urine.

In some embodiments, the bio-physical or physical property is a non-cancerous signal and exists in the biological subject from a healthy individual.

In some embodiments, the bio-physical or physical property exists and can be detected in both the biological subject from a healthy individual, the biological subject from a non-cancer disease patient, and the biological subject from a patient with each of the two or more types of cancer; and the detected properties between the healthy individual, the non-cancer disease patient, and the patient with the cancer are different.

In some embodiments, the bio-physical or physical property exits in each of the two or more types of cancer, and is able to differentiate a normal sample from abnormal samples with the cancers.

In some embodiments, the determination is by comparing the biophysical information of the detected biological subject with the same biological information of a confirmed disease-free or diseased biological subject.

In some embodiments, the status of each of the cancers comprises health stage, non-cancer disease stage, pre-cancer stage, early stage cancer stage, and mid to late stage cancer stage, with statistically significant detection or monitoring between any of the two stages.

In some other embodiments, the apparatus simultaneously can presence or monitoring progression of three or more types of cancer in the biological subject.

In some embodiments, the cancer types may comprise lung cancer, liver cancer, colorectal cancer, breast cancer, gastric cancer, esophageal cancer, brain tumor, prostate cancer, blood cancer, intestine cancer, stomach cancer, cervical cancer, ovarian cancer, rectum cancer, colon cancer, nasopharyngeal cancer, cardiac carcinoma, uterine cancer, oophoroma, pancreatic cancer, or circulating tumor cells.

In some other embodiments, an additional device is used for adjusting the physical or bio-physical property in the biological subject. For instance, the physical or bio-physical property may be first measured and then adjusted.

In some embodiments, such physical or bio-physical property comprises a mechanical property, an acoustical property, an optical property, an electrical property, an electro-magnetic property, or an electro-mechanical property. More specifically, the electrical property may comprise electrical current, electrical conductance, capacitance, electrical resistance, ion types and/or concentrations, or quantum mechanical effect.

In some embodiments, the additional device adjusts the current to a higher value, adjusts the electrical conductance to a higher value, adjusts the electrical resistance to a lower value, or alters the quantum mechanical effect.

In some embodiments, a reagent is injected into blood to adjust bio-physical properties in the blood. For example, the reagent may contain ions, oxidizers, and components to impacting electrical properties of the blood. Such electrical property may comprise electrical current, electrical conductance, capacitance, electrical resistance, ion types and/or concentrations, or quantum mechanical effect.

In some embodiments, the reagent is a drug capable of adjusting the biological properties in the blood. In some further embodiments, the drug is capable of releasing, upon intake, ions and charged components and capable of adjusting electrical properties of the blood. Such property may comprise electrical current, electrical conductance, capacitance, electrical resistance, ion types and/or concentrations, or quantum mechanical effect.

In some other embodiments, at least one bio-marker is added to the liquid sample for physical or bio-physical property and related properties to be measured. In some embodiments, the bio-marker provides at least some indicative information of risks of two or more types of cancer occurrence at a given organ and location.

In still some other embodiments, the detected property is analyzed in conjunction with information and data obtained from test(s) comprising of bio-marker tests, genomics tests, circulating tumor DNA, circulating free tumor DNA, and circulating tumor cell tests, and overall cancer risks and location(s) of possible cancer occurrence are obtained.

In some embodiments, the apparatus comprises an ion injector, configured to add a desired amount of ions to the biological subject. For instance, the ions may comprise potassium ions, sodium ions, chloride ions, nitride ions, or calcium ions. Particularly, the ions may include potassium ions.

In some embodiments, the apparatus comprises one or more channels, and the one or more channels comprise one or more detectors; wherein the one or more detectors are configured to detect one or more bio-physical or physical properties of the biological subject.

In some embodiments, the apparatus further comprises one or more ion injectors on sidewalls, and each of the one or more ion injectors is configured to add a desired amount of ions to the biological subject. The ions added by ion injectors can be the same or different; and the bio-physical or physical properties detected by different detectors can be the same or different.

In some embodiments, the detector comprises one or more sensors placed partially or completely alongside one or more side walls of the channel or chamber. Each sensor may be independently a thermal sensor, optical sensor, acoustical sensor, biological sensor, chemical sensor, electro-mechanical sensor, electro-chemical sensor, electro-optical sensor, electro-thermal sensor, electro-chemical-mechanical sensor, bio-chemical sensor, bio-mechanical sensor, bio-optical sensor, electro-optical sensor, bio-electro-optical sensor, bio-thermal optical sensor, electro-chemical optical sensor, bio-thermal sensor, bio-physical sensor, bio-electro-mechanical sensor, bio-electro-chemical sensor, bio-electro-optical sensor, bio-electro-thermal sensor, bio-mechanical-optical sensor, bio-mechanical thermal sensor, bio-thermal-optical sensor, bio-electro-chemical-optical sensor, bio-electro-mechanical optical sensor, bio-electro-thermal-optical sensor, bio-electro-chemical-mechanical sensor, physical sensor, mechanical sensor, piezo-electrical sensor, piezo-electro photronic sensor, piezo-photronic sensor, piezo-electro optical sensor, bio-electrical sensor, bio-marker sensor, electrical sensor, magnetic sensor, electromagnetic sensor, image sensor, or radiation sensor.

More specifically, the thermal sensor may include a resistive temperature micro-sensor, a micro-thermocouple, a thermo-diode and thermo-transistor, and a surface acoustic wave (SAW) temperature sensor; the image sensor may include a charge coupled device (CCD) or a CMOS image sensor (CIS); the radiation sensor may include a photoconductive device, a photovoltaic device, a pyro-electrical device, or a micro-antenna; the mechanical sensor may include a pressure micro-sensor, micro-accelerometer, flow meter, viscosity measurement tool, micro-gyrometer, or micro flow-sensor; the magnetic sensor may include a magneto-galvanic micro-sensor, a magneto-resistive sensor, a magneto diode, or magneto-transistor; the biochemical sensor may include a conductimetric device, a bio-marker, a bio-marker attached to a probe structure, or a potentiometric device.

In some embodiments, at least one sensor is a probing sensor and applies a probing or disturbing signal to the biological subject. In some further embodiments, at least another sensor, different from the probing sensor, is a detection sensor and detects a response from the biological subject upon which the probing or disturbing signal is applied.

In some other embodiments, a read-out circuitry is connected to at least one sensor and transfers data from the sensor to a recording device. The connection between the read-out circuit and the sensor may be digital, analog, optical, thermal, piezo-electrical, piezo-photronic, piezo-electrical photronic, opto-electrical, electro-thermal, opto-thermal, electric, electromagnetic, electromechanical, or mechanical.

In some embodiments, the chamber or channel has a length ranging from 1 micron to 50,000 microns, from 1 micron to 15,000 micron, from 1 micron to 10,000 microns, from 1.5 microns to 5,000 microns, or from 3 microns to 1,000 microns.

In some embodiments, the chamber or channel has a width or height ranging from 0.1 micron to 100 microns; from 0.1 micron to 25 microns, from 1 micron to 15 microns, or from 1.2 microns to 10 microns.

In some embodiments, at least four sensors are located on one side, two opposite sides, or four sides of the interior surface of the chamber or channel.

In some embodiments, an application specific integrated circuit chip is internally bonded to or integrated into the detector.

In some embodiments, the apparatus is fabricated by integrated circuit technologies.

Another aspect of this invention relates to a method for screening or detecting the presence or progression of two or more types of cancer in a biological subject, comprising measuring a physical or biophysical property at a microscopic level of cells in the biological subject with an apparatus of this invention, wherein information related to the measured property of the cells in the biological subject is detected by the detector and collected for analysis to determine whether each of the cancers is likely to be present with the biological subject or to determine the status of each cancer, thereby providing the ability to simultaneously determine or monitor progression of the two or more types of cancer.

In some embodiments, the measured property is collected for analysis to simultaneously determine or monitor progression of three or more types of cancer.

Still a further aspect of this invention provides an apparatus for treating a biological subject, comprising: a channel or chamber in which the biological subject passes through; and at least one transducer placed partially or completely in the channel or chamber; wherein the transducer is configured to transmit at least one bio-physical property, bio-physical energy, material or element onto the biological subject, thereby providing a simultaneous treatment of two or more types of cancer in the biological subject.

In some embodiments, the biological subject is a liquid sample of a mammal. The biological subject may be a blood sample, a urine sample, or a sweat sample of the mammal. More specifically, the biological subject may comprise blood, proteins, red blood cells, while blood cells, T cells, other cells, gene mutations, quantum mechanical effects, DNA, RNA, or other biological entities.

In some embodiments, the cancers comprise lung cancer, liver cancer, colorectal cancer, breast cancer, gastric cancer, esophageal cancer, brain tumor, prostate cancer, blood cancer, intestine cancer, stomach cancer, cervical cancer, ovarian cancer, rectum cancer, colon cancer, nasopharyngeal cancer, cardiac carcinoma, uterine cancer, oophoroma, pancreatic cancer, or circulating tumor cells.

In some embodiments, the at least one bio-physical property, bio-physical energy, material or element comprises a mechanical property or energy, an acoustical property or energy, an optical property or energy, an electrical property or energy, an electro-magnetic property or energy, or an electro-mechanical property or energy.

In some embodiments, the at least one electrical property or energy comprises electrical current, electrical voltage, electrical field, electro-magnetic field, electrical conductance, capacitance, electrical resistance, net electrical charge in extracellular region, membrane potential, membrane polarization, membrane ion channel properties, resting membrane potential, trans-membrane potential, depolarized trans-membrane potential, ion concentrations, electro-static force and charge on DNA double helix and RNA double helix, or quantum mechanical effect.

In some embodiments, the at least one bio-physical property, bio-physical energy, material or element comprises a trans-membrane potential, a membrane voltage, a membrane potential, a membrane ion channel property, resting membrane potential, trans-membrane potential, depolarized trans-membrane potential, a zeta potential, an impedance, an optical reflective index, an optical refractive index, potassium ions, sodium ions, chloride ions, nitride ions, calcium ions, an electro-static force, an electro-static force acting on cells, an electro-static force acting on DNA double helix, an electro-static force acting on RNA, an electrical charge on cell membrane, an electrical charge on DNA double helix, an electrical charge on RNA, quantum effects, near-field electrical properties, near-field electro-magnetic properties, membrane bilayer properties, ion types and/or concentrations, ion permeability, electrical current, electrical conductance, capacitance, or electrical resistance.

In some embodiments, the transmitted bio-physical property or energy adjusts the current of the biological subject to a higher value, adjusts the electrical conductance of the biological subject to a higher value, adjusts the electrical resistance of the biological subject to a lower value, or alters the quantum mechanical effect of the biological subject.

In some embodiments, the at least one transducer is placed alongside a side wall of the channel or chamber, and is configured to apply a pulsed electrical voltage to the biological subject passing through the channel.

In some further embodiments, the biological subject is the blood sample, and the applied voltage is configured to impact an electrical field, charge distribution, membrane ion channel properties, resting membrane potential, trans-membrane potential, depolarized trans-membrane potential, or membrane potential of the blood sample.

In some other embodiments, the transducer is a generator configured to apply at least one type of energy or field onto the biological subject.

In some further embodiment, the generator is looped around the channel in which the biological subject flows through or stays statically for a desired period of time. The energy may comprise physical energy, bio-physical energy, bio-chemical energy, electrical energy, electro-magnetic energy, magnetic energy, optical energy, acoustic energy, thermal energy, mechanical energy, gravitational field energy, quantum mechanical energy, or radiation energy. Also, the energy can be applied in constant, alternating, or pulsed form.

In some embodiments, the channel applying the energy is a coil surrounding channel.

In some embodiments, the apparatus comprises at least one optical energy generator, acoustic energy generator, mechanical force generator, gravitational field generator, quantum mechanical field generator, electrical field generator, electro-magnetic field generator, electrical voltage generator, thermal energy generator, or radiation energy generator mounted along the channel.

In some embodiments, the field comprises electrical field, magnetic field, electro-magnetic field, quantum field, mechanical force field, or gravitational field.

In still some other embodiments, the apparatus includes at least one ion injector connecting to the channel, wherein the ion injector is configured to add a desired amount of ions to the biological subject.

In some embodiments, the apparatus comprises one or more channels, and the one or more channels comprise one or more transducers on sidewalls, and one or more ion injectors connecting to the one or more channels, optionally through one or more small openings; wherein at least one transducer is configured to transmit a bio-physical energy to the biological subject, and the at least one ion injector is configured to add a desired amount of ions to the biological subject. In some further embodiments, the biological subject can be the blood sample. Moreover, the bio-physical energy can be an electrical pulse; and the added ions may comprise potassium ions. Such an apparatus can enhance an electrical conductivity of the blood sample, a net ion concentration, an electrolyte concentration, a net cell surface charge, a net DNA surface charge, a net RNA surface charge, a net protein surface charge, a net electrical charge in the blood sample, membrane ion channel properties, resting membrane potential, trans-membrane potential, depolarized trans-membrane potential, a membrane potential, a membrane voltage, or a polarization of membrane potential.

In some embodiments, the apparatus further includes at least one detector placed partially or completely alongside one or more side walls of the channel or chamber, wherein the at least one detector is configured to detect at least one bio-physical or physical property of the biological subject.

In some embodiments, the bio-physical or physical signal exist and can be detected in both the biological subject from a healthy individual, the biological subject from a non-cancer disease patient, and the biological subject from a patient with any of the cancers to be treated; and the detected signals between the healthy individual, the non-cancer disease patient, and the patient with the cancers are different.

In some embodiments, the apparatus comprises one or more channels, and one or more detectors, one or more ion injectors, and one or more generators in or around the channels; wherein the one or more generators are configured to apply at least one type of energy or field onto the biological subject; the one or more detectors are configured to detect one or more bio-physical or physical properties of the biological subject, and each of the one or more ion injectors is configured to add a desired amount of ions to the biological subject.

In some embodiments, the apparatus is capable of simultaneously impacting two or more cancer statuses, each involving converting at least a property of the biological subject from a cancer state to an earlier cancer stage, a pre-cancer state, a non-cancer disease state or a healthy state; converting at least one property of the biological subject from a pre-cancer state to a non-cancer disease state or healthy state; or converting at least one property of the biological subject from a non-cancer disease state to a healthy state.

In some embodiments, the property comprises a physical property, bio-physical property, bio-chemical property, protein property, cellular property, molecular property, genomic property, or immunology property. In some other embodiments, the property comprises an optical property, acoustical property, thermal property, quantum property, gravitational property, mechanical property, electrical property, magnetic property, or electro-magnetic property.

In some embodiments, the apparatus is configured to impact at least one of the following properties in the biological subject: electrolyte concentration and distribution, potassium ion concentration and distribution, sodium ion concentration and distribution, chloride ion concentration and distribution, calcium ion concentration and distribution, net charge in extracellular region, ion concentration in extracellular, gravitational field, mechanical field, quantum mechanical field, magnetic field, electro-magnetic field, electrical field, electrical current, electrical resistance, electrical conductance, capacitance, membrane ion channel properties, resting membrane potential, trans-membrane potential, depolarized trans-membrane potential, membrane voltage, and membrane potential.

In some embodiments, the apparatus is fabricated by integrated circuit technologies. The integrated circuit technologies may comprise thin film deposition, lithography, etch, diffusion, ion implantation, annealing, cleaning, or polishing processes.

In some embodiments, the apparatus comprises semiconductor, electrically insulating, and electrically conductive materials. More specifically, the apparatus may comprise a material selected from group consisting of silicon, germanium, glass, silicon dioxide, silicon nitride, poly-silicon, tungsten, aluminum, copper, gold, and silicon carbide.

Also within the scope of this invention is a method for treating or slowing progression of two or more types of cancer in a patient, comprising administering to the patient thereof a therapeutic agent that enhances or increase the level of a biophysical property at the microscopic level of the patient, wherein the level of the biophysical property simultaneously impacts the status of each of the two or more type cancers.

In some embodiments, the therapeutic agent is administered orally or by intravenous injection.

In some embodiments, the biophysical property is an electronic property. For instance, the electronic property is surface charge, surface potential, resting potential, electrical current, electrical field distribution, surface charge distribution, cell electronic properties, cell surface electronic properties, dynamic changes in electronic properties, dynamic changes in cell electronic properties, dynamic changes in cell surface electronic properties, dynamic changes in surface electronic properties, membrane ion channel properties, resting membrane potential, trans-membrane potential, depolarized trans-membrane potential, electronic properties of cell membranes, dynamic changes in electronic properties of membrane surface, dynamic changes in electronic properties of cell membranes, electrical dipole, electrical quadruple, oscillation in electrical signal, electrical current, capacitance, three-dimensional electrical or charge cloud distribution, electrical properties at telomere of DNA and chromosome, DNA surface charge, DNA surrounding media electrical properties, quantum mechanical effects, capacitance, or impedance.

In some embodiments, physical or bio-physical detection method may be combined with other detection methods to achieve unique and much more comprehensive detection performance results. Examples of other detection methods to be combined include but not limited to bio-chemistry, immunological, genomic, circulating tumor cell, and imaging detection technologies. Physical or bio-physical detection tends to be simpler and cost effective tests, which gives more fundamental, earlier and overall (more global in terms of covering common properties of more types of cancer/tumor, such as brain tumor and esophageal cancer) information about cancer occurrence, while other tests tend to be more complex and provide additional, more specific information - such as more specific and/or detailed information about a given type of cancer and its location. The combination tests can yield non-linearly more information. For instance, physical or bio-physical cancer detection method can be combined with bio-marker tests to obtain both (a) earlier stage cancer or pre-cancer information (with physical or bio-physical detection method), as well as more specific cancer type information including location or organ information (with bio-marker tests). In an immunological measurement, bio-markers may include alpha-fetoprotein, prostate specific antigen, arcinoembryonic antigen, carbohydrate antigen 50, carbohydrate antigen 242, carbohydrate antigen 125, carbohydrate antigen 153, carbohydrate antigen 199, and carbohydrate antigen 724. The above combination test approach can be called platform approach. In this work, it is called CDA (cancer differentiation analysis) platform, while physical or bio-physical detection approach alone is called CDA method.

In some embodiments, the cancers may include lung cancer, liver cancer, colorectal cancer, breast cancer, gastric cancer, esophageal cancer, brain tumor, prostate cancer, blood cancer, intestine cancer, stomach cancer, cervical cancer, ovarian cancer, rectum cancer, colon cancer, nasopharyngeal cancer, cardiac carcinoma, uterine cancer, oophoroma, pancreatic cancer, or circulating tumor cells.

In still another aspect, this invention provides a therapeutic agent for treating or slowing progression of two or more types of cancer in a patient, comprising a component that alters or enhances an electronic property of the patient.

Examples of the component include electrolytes or components which release electrolytes. The component may enhance electrical current, electro-static charges on DNA surfaces, and/or electrical conductance, reduces electrical resistance, and/or alters quantum mechanical effect.

The present invention can detect the presence of multiple types of diseases, including multiple types of cancer, with a single test. Examples of cancer comprise breast cancer, lung cancer, esophageal cancer, intestine cancer, cancer related to blood, liver cancer, stomach cancer, cervical cancer, ovarian cancer, rectum cancer, colon cancer, nasopharyngeal cancer, cardiac carcinoma, uterine cancer, oophoroma, pancreatic cancer, prostate cancer, brain tumor, and circulating tumor cells; examples of the inflammatory disease include acne vulgaris, asthma, autoimmune diseases, autoinflammatory diseases, celiac disease, chronic prostatitis, diverticulitis, glomerulonephritis, hidradenitis suppurativa, hypersensitivities, inflammatory bowel diseases, interstitial cystitis, otitis, pelvic inflammatory disease, reperfusion injury, rheumatic fever, rheumatoid arthritis, sarcoidosis, transplant rejection, and tasculitis; examples of the lung disease include asthma, chronic obstructive pulmonary disease, chronic bronchitis, emphysema, acute bronchitis, cystic fibrosis, pneumonia, tuberculosis, pulmonary edema, acute respiratory distress syndrome, pneumoconiosis, interstitial lung disease, pulmonary embolism, and pulmonary hypertension; examples of the diabetes include Type 1 diabetes, Type 2 diabetes, and gestational diabetes; examples of the heart disease include coronary artery disease, enlarged heart (cardiomegaly), heart attack, irregular heart rhythm, atrial fibrillation, heart rhythm disorders, heart valve disease, sudden cardiac death, congenital heart disease, heart muscle disease (cardiomyopathy), dilated cardiomyopathy, hypertrophic cardiomyopathy, restrictive cardiomyopathy, pericarditis, pericardial effusion, marfan syndrome, and heart murmurs; examples of the liver disease include fascioliasis, hepatitis, alcoholic liver disease, fatty liver disease (hepatic steatosis), hereditary diseases, Gilbert's syndrome, cirrhosis, primary biliary cirrhosis, primary sclerosing cholangitis, and Budd-Chiari syndrome; examples of the gastric disease include gastritis, gastric polyp, gastric ulcer, benign tumor of stomach, acute gastric mucosa lesion, antral gastritis, and gastric stromal tumors; examples of the biliary disease include calculus of bile duct, cholecystolithiasis, cholecystitis, cholangiectasis, cholangitis, and gallbladder polyps; the cardiovascular disease comprises coronary artery disease, peripheral arterial disease, cerebrovascular disease, renal artery stenosis, aortic aneurysm, cardiomyopathy, hypertensive heart disease, heart failure, pulmonary heart disease, cardiac dysrhythmias, endocarditis, inflammatory cardiomegaly, myocarditis, valvular heart disease, congenital heart disease, rheumatic heart disease, coronary artery disease, peripheral arterial disease, cerebrovascular disease, and renal artery stenosis.

A further aspect of this invention provides an apparatus for treating a disease of the biological subject, comprising a coil surrounding channel, in which the biological subject passes through, wherein the coil surrounding channel is configured to apply at least one type of energy or field onto the biological subject.

Ins some embodiments, the coil surrounding channel comprises a multiple-layer structure. For instance, the coil may comprise: a top conductive layer, a middle layer further comprising conductive plugs, and a bottom conductive layer; wherein the top conductive layer, the conductive plugs, and the bottom conductive layer are surrounded by one or more insulating materials; and the conductive plugs connects the top conductive layer and the bottom conductive layer, thereby forming the coil surrounding the channel.

In some embodiments, the apparatus simultaneously treats two or more types of cancer. Examples of the cancers include, but are not limited to, lung cancer, liver cancer, colorectal cancer, breast cancer, gastric cancer, esophageal cancer, brain tumor, prostate cancer, blood cancer, intestine cancer, stomach cancer, cervical cancer, ovarian cancer, rectum cancer, colon cancer, nasopharyngeal cancer, cardiac carcinoma, uterine cancer, oophoroma, pancreatic cancer, or circulating tumor cells.

In some embodiments, the energy comprises physical energy, bio-physical energy, bio-chemical energy, electrical energy, electro-magnetic energy, magnetic energy, optical energy, acoustic energy, thermal energy, mechanical energy, gravitational field energy, quantum mechanical energy, or radiation energy. The energy can be applied in constant, alternating, or pulsed form.

In some embodiments, the field comprises electrical field, magnetic field, electro-magnetic field, quantum field, mechanical force field, or gravitational field.

In some embodiments, the apparatus further includes at least one detector placed partially or completely in the channel, wherein the at least one detector is configured to detect at least one bio-physical or physical signal of the biological subject; and an ion injector connecting to the channel, wherein the ion injector is configured to add a desired amount of ions to the biological subject.

In some embodiments, the apparatus includes one or more coil surrounding channels, one or more detectors, and one or more ion injectors; wherein the one or more coil surrounding channels are configured to apply at least one type of energy or field onto the biological subject; the one or more detectors are configured to detect one or more bio-physical or physical properties of the biological subject, and each of the one or more ion injectors is configured to add a desired amount of ions to the biological subject.

In some embodiments, the apparatus is capable of simultaneously impacting two or more cancer statuses, each involving converting at least a property of the biological subject from a cancer state to an earlier cancer stage, a pre-cancer state, a non-cancer disease state or a healthy state; converting at least one property of the biological subject from a pre-cancer state to a non-cancer disease state or healthy state; or converting at least one property of the biological subject from a non-cancer disease state to a healthy state.

For instance, the property comprises a physical property, bio-physical property, bio-chemical property, protein property, cellular property, molecular property, genomic property, or immunology property. The property may also comprise an optical property, acoustical property, thermal property, gravitational property, mechanical property, quantum property, electrical property, magnetic property, or electro-magnetic property.

In some embodiments, the apparatus is configured to impact at least one of the following properties in the biological subject: electrolyte concentration and distribution, potassium ion concentration and distribution, sodium ion concentration and distribution, chloride ion concentration and distribution, calcium ion concentration and distribution, net charge in extracellular region, ion concentration in extracellular, gravitational field, mechanical field, quantum mechanical field, magnetic field, electro-magnetic field, electrical field, electrical current, electrical resistance, electrical conductance, capacitance, and membrane ion channel properties, resting membrane potential, trans-membrane potential, depolarized trans-membrane potential, membrane voltage, membrane potential.

The biological subject can be a liquid sample of a mammal, such as a blood sample, a urine sample, or a sweat sample of the mammal. Such a biological subject may include blood, proteins, red blood cells, while blood cells, T cells, other cells, gene mutations, quantum mechanical effects, DNA, RNA, or other biological entities.

In some embodiments, the apparatus is fabricated by integrated circuit technologies, including, e.g., thin film deposition, lithography, etch, diffusion, ion implantation, annealing, cleaning, or polishing processes. The apparatus may be made of semiconductor, electrically insulating, and/or electrically conductive materials.

Still within the scope of this invention is a method of fabricating a micro-device for treating a disease, wherein the method uses microelectronics process and comprises the following step: fabricating a coil surrounding channel by semiconductor or integrated circuit process, wherein the coil surrounding channel is configured to apply at least one type of energy or field onto a biological subject passing through or staying in the channel.

For instance, the method may comprise the following steps: providing a substrate; depositing material A onto the surface region of the substrate, and patterning or etching material A to form first recessed areas; depositing material B onto the surface region of material A and filing into the first recessed areas; etching or polishing material B to remove material B from the top surface of material A and leaving sufficient amount of material B in the recessed areas coplanar with the top surface of material A; depositing material C onto the surfaces of materials A and B to form a thin layer; depositing layer material D onto the surface of material C, and patterning material D to form second recessed areas; depositing sacrificial material E, and etching or polishing material E to remove material E from the top surface of material D and leaving sufficient amount of material E in the second recessed areas coplanar with the top surface of material D; depositing material F onto the surfaces of materials D and E, and using an etching process to etch a small hole through material F; using a further etching process to etch off sacrificial material E, thereby forming micro-channels in material D, depositing material G onto the surface of material F to form a thin etch stop layer; depositing material H onto the surface of material G; using pattering and etching processes to form trench areas in material H, and deep hole areas through materials G, F, D, and C stopping on the top surface of material B; and depositing material I into the trench areas and the deep hole areas, thereby contacting material B, wherein material I and material B together form a coil, which surrounds the micro-channels formed in material D.

In some embodiments, the substrate is a semiconductor or insulating substrate.

In some embodiments, material A is an insulating material. Material B can be a conductive material, and can be polished via chemical mechanical polishing (CMP). Material C may comprise silicon nitride or poly-silicon.

In some embodiments, material D may be an insulating material, optionally being the same as material A. In some embodiments, material E is polished via chemical mechanical polishing (CMP).

Still in some embodiments, material F is an insulating material, optionally same as material A. Materials A, D, and F may be the same insulating material. Material G may also comprise silicon nitride or poly-silicon, and optionally being the same as material C.

In some embodiments, material H is an insulating material, optionally the same material as material A. Materials A, D, F, and H may be the same material.

In some embodiments, the formation of the trench areas and the deep hole areas further comprises: patterning and etching material H to form the trench areas, and then further patterning and etching to form the deep hole areas through materials G, F, D, and C, stopping on the top surface of material B. For instance, the hole area is etched by different etch chemistries, e.g., with one chemistry for materials G and C, and another chemistry for materials F and D.

Alternatively, the trench areas and the deep hole areas are formed by using a double lithography exposure, for trench area and deep hole area respectively, and doing a double-etch, thereby etching the trench area first and then the hole area.

In some embodiments, material I is a conductive material, optionally being the same as material B.

Still in another aspect, the present application provides a method of fabricating a micro device with a coil surrounding channel, comprising the following steps: providing a substrate; depositing material A1 onto the surface region of the substrate, and patterning or etching material A1 to form first recessed areas; depositing material B1 onto the surface region of material A1 and filing into the first recessed areas; etching or polishing material B1 to remove material B1 from the top surface of material A1 and leaving sufficient amount of material B1 in the recessed areas coplanar with the top surface of material A1 ; depositing material C1 onto the surfaces of materials A1 and B1 to form a thin layer; depositing layer material D1 onto the surface of material C1 , and patterning material D1 to form second recessed areas; depositing sacrificial material E1 , and etching or polishing material E1 to remove material E from the top surface of material D1 and leaving sufficient amount of material E1 in the second recessed areas coplanar with the top surface of material D1 ; depositing material F onto the surfaces of materials D1 and E1, and using an etching process to etch a small hole through material F1; using a further etching process to etch off sacrificial material E1 , thereby forming micro-channels in material D1, depositing material G1 onto the surface of material F1 to form a thin etch stop layer; depositing material H1 onto the surface of material G1 ; using pattering and etching processes to form trench areas in material H1, and deep hole areas through materials G1, F1, D1, and C1 stopping on the top surface of material B1; and depositing material I1 into the trench areas and the deep hole areas, thereby contacting material B, wherein material I1 and material B1 together form a coil, which surrounds the micro-channels formed in material D1.

In some embodiments, the coil surrounding channel is configured to apply at least one type of energy or field onto a biological subject passing through or staying in the channel. In some embodiments, the substrate is a semiconductor or insulating substrate.

In some embodiments, each of materials A1 , D1, F1, and H1 is an insulating material (optionally the same material). Each of materials B1 and I1 can be a conductive material (optionally the same material). At least one of materials C1 and G1 may comprise nitride or poly-silicon. Materials C1 and G1 may be the same.

In some embodiments, at least one of materials B1 and El is polished via chemical mechanical polishing (CMP).

In some embodiments, the formation of the trench areas and the deep hole areas further comprises: patterning and etching material H1 to form the trench areas, and then further patterning and etching to form the deep hole areas through materials G1, F1, D1, and C1, stopping on the top surface of material B 1. The hole area may be etched by different etch chemistries, e.g., with one chemistry for materials G1 and C1, and another chemistry for materials F1 and D1.

Alternatively, the trench areas and the deep hole areas are formed by using a double lithography exposure, for trench area and deep hole area respectively, and doing a double-etch, thereby etching the trench area first and then the hole area.

As used herein, the term “biomarker” means a measurable indicator of the severity or presence of some disease state, but more generally a biomarker is anything that can be used as an indicator of a particular disease state or some other physiological state of an organism. A biomarker can be a substance that is introduced into an organism as a means to examine organ function or other aspects of health. For example, rubidium chloride is used in isotopic labeling to evaluate perfusion of heart muscle. It can also be a substance whose detection indicates a particular disease state, for example, the presence of an antibody may indicate an infection. More specifically, a biomarker indicates a change in expression or state of a protein that correlates with the risk or progression of a disease, or with the susceptibility of the disease to a given treatment. Biomarkers can be specific cells, molecules, or genes, gene products, enzymes, or hormones.

As used herein, the term “or” is meant to include both “and” and “or”. It may be interchanged with “and/or.”

As used herein, a singular noun is meant to include its plural meaning. For instance, a micro device can mean either a single micro device or multiple micro-devices.

As used herein, the term “patterning” means shaping a material into a certain physical form or pattern, including a plane (in which case “patterning” would also mean “planarization”).

As used herein, the term “a biocompatible material” refers to a material that is intended to interface with a living organism or a living tissue and can function in intimate contact therewith. When used as a coating, it reduces the adverse reaction a living organism or a living tissue has against the material to be coated, e.g., reducing the severity or even eliminating the rejection reaction by the living organism or living tissue. As used herein, it encompasses both synthetic materials and naturally occurring materials. Synthetic materials usually include biocompatible polymers, made either from synthetic or natural starting materials, whereas naturally occurring biocompatible materials include, e.g., proteins or tissues.

As used herein, the term “a biological subject” or “a biological sample” for analysis or test or diagnosis refers to the subject to be analyzed by a disease detection apparatus. It can be a single cell, a single biological molecular (e.g., DNA, RNA, or protein), a single biological subject (e.g., a single cell or virus), any other sufficiently small unit or fundamental biological composition, a sample of a subject's or patient's organ or tissue that may having a disease or disorder, or a patient.

As used herein, the term “disease” is interchangeable with the term “disorder” and generally refers to any abnormal microscopic property or condition (e.g., a physical condition) of a biological subject (e.g., a mammal or biological species).

As used herein, the term “subject” generally refers to a mammal, e.g., a human person.

As used herein, the term “microscopic level” refers to the subject being analyzed by the disease detection apparatus of this invention is of a microscopic nature and can be a single cell, a single biological molecular (e.g., DNA, RNA, or protein), a single biological subject (e.g., a single cell or virus), and other sufficiently small unit or fundamental biological composition.

As used herein, an “apparatus” or a “micro-device” or “micro device” can be any of a wide range of materials, properties, shapes, and degree of complexity and integration. The term has a general meaning for an application from a single material to a very complex device comprising multiple materials with multiple sub units and multiple functions. The complexity contemplated in the present invention ranges from a very small, single particle with a set of desired properties to a fairly complicated, integrated unit with various functional units contained therein. For example, a simple micro-device could be a single spherical article of manufacture of a diameter as small as 100 angstroms with a desired hardness, a desired surface charge, or a desired organic chemistry absorbed on its surface. A more complex micro device could be a 1 millimeter device with a sensor, a simple calculator, a memory unit, a logic unit, and a cutter all integrated onto it. In the former case, the particle can be formed via a fumed or colloidal precipitation process, while the device with various components integrated onto it can be fabricated using various integrated circuit manufacturing processes. In some places, a micro-device or micro device represents a sub-equipment unit.

As used herein, the term “parameter” refers to a particular detection target (e.g., a property of microscopic level, physical property such as hardness, viscosity, current, or voltage, or chemical property such as pH value) of the biological subject to be detected, and can include micro-level property.

As used herein, the term “level” refers to chemical composition (including biochemical composition such as protein, genetic materials, e.g., DNA and RNA), cellular classification, or molecular classification of the biological subject to be detected.

As used herein, the term “component” refers a lower division or building block of a level described above. For instance, a protein level can include such components as alpha-feto protein or sugar protein; and the level of a cellular classification can include such components as surface voltage and membrane composition.

As used herein, if not specifically defined, a “channel” or “chamber” can be either an inter-unit channel or an intra-unit channel.

Biological subjects that can be detected by the apparatus include, e.g., blood, urine, saliva, tear, and sweat. The detection results can indicate the possible occurrence or presence of a disease (e.g., one in its early stage) in the biological subject.

As used herein, the term “absorption” typically means a physical bonding between the surface and the material attached to it (absorbed onto it, in this case). On the other hand, the word “adsorption” generally means a stronger, chemical bonding between the two. These properties are very important for the present invention as they can be effectively used for targeted attachment by desired micro devices for measurement at the microscopic level.

As used herein, the term “contact” (as in “the first micro-device contacts a biologic entity”) is meant to include both “direct” (or physical) contact and “non-direct” (or indirect or non-physical) contact. When two subjects are in “direct” contact, there is generally no measurable space or distance between the contact points of these two subjects; whereas when they are in “indirect” contact, there is a measurable space or distance between the contact points of these two subjects.

As used herein, the term “probe” or “probing,” in addition to its dictionary meaning, could mean applying a signal (e.g., an acoustic, optical, magnetic, chemical, electrical, electro-magnetic, bio-chemical, bio-physical, or thermal signal) to a subject and thereby stimulating the subject and causing it to have some kind of intrinsic response.

As used herein, the term “thermal property” refers to temperature, freezing point, melting point, evaporation temperature, glass transition temperature, or thermal conductivity.

As used herein, the term “optical property” refers to reflection, optical absorption, optical scattering, wave length dependent properties, color, luster, brilliance, scintillation, or dispersion.

As used herein, the term “electrical property” refers to surface charge, surface potential, electrical field, charge distribution, electrical field distribution, resting potential, action potential, or impedance of a biological subject to be analyzed.

As used herein, the term “magnetic property” refers to diamagnetic, paramagnetic, or ferromagnetic.

As used herein, the term “electromagnetic property” refers to property that has both electrical and magnetic dimensions.

As used herein, the term “acoustical property” refers to the characteristics found within a structure that determine the quality of sound in its relevance to hearing. It can generally be measured by the acoustic absorption coefficient. See, e.g., U.S. Pat. No. 3,915,016, for means and methods for determining an acoustical property of a material; T. J. Cox et al., Acoustic Absorbers and Diffusers, 2004, Spon Press.

As used herein, the term “biological property” is meant to generally include chemical and physical properties of a biological subject.

As used herein, the term “chemical property” refers to pH value, ionic strength, or bonding strength within the biological sample.

As used herein, the term “physical property” refers to any measurable property the value of which describes a physical system's state at any given moment in time. The physical properties of a biological sample may include, but are not limited to absorption, albedo, area, brittleness, boiling point, capacitance, color, concentration, density, dielectrical, electrical charge, electrical conductivity, electrical impedance, electrical field, electrical potential, emission, flow rate, fluidity, frequency, inductance, intrinsic impedance, intensity, irradiance, luminance, luster, malleability, magnetic field, magnetic flux, mass, melting point, momentum, permeability, permittivity, pressure, radiance, solubility, specific heat, strength, temperature, tension, thermal conductivity, flow rate, velocity, viscosity, volume, surface area, shape, and wave impedance.

As used herein, the term “mechanical property” refers to strength, hardness, flow rate, viscosity, toughness, elasticity, plasticity, brittleness, ductility, shear strength, elongation strength, fracture stress, or adhesion of the biological sample.

As used herein, the term “disturbing signal” has the same meaning as “probing signal” and “stimulating signal.”

As used herein, the term “disturbing unit” has the same meaning as “probing unit” and “stimulating unit.”

As used herein, the term “conductive material” (or its equivalent “electrical conductor”) is a material which contains movable electrical charges. A conductive material can be a metal (e.g., copper, silver, or gold) or non-metallic (e.g., graphite, solutions of salts, plasmas, or conductive polymers). In metallic conductors, such as copper or aluminum, the movable charged particles are electrons (see electrical conduction). Positive charges may also be mobile in the form of atoms in a lattice that are missing electrons (known as holes), or in the form of ions, such as in the electrolyte of a battery.

As used herein, the term “electrically insulating material” (also known as “insulator” or “dielectric”) refers to a material that resists the flow of electrical current. An insulating material has atoms with tightly bonded valence electrons. Examples of electrically insulating materials include glass or organic polymers (e.g., rubber, plastics, or Teflon).

As used herein, the term “semiconductor” (also known as “semiconducting material”) refers to a material with electrical conductivity due to electron flow (as opposed to ionic conductivity) intermediate in magnitude between that of a conductor and an insulator. Examples of inorganic semiconductors include silicon, silicon-based materials, and germanium. Examples of organic semiconductors include such aromatic hydrocarbons as the polycyclic aromatic compounds pentacene, anthracene, and rubrene; and polymeric organic semiconductors such as poly(3-hexylthiophene), poly(p-phenylene vinylene), polyacetylene and its derivatives. Semiconducting materials can be crystalline solids (e.g., silicon), amorphous (e.g., hydrogenated amorphous silicon and mixtures of arsenic, selenium and tellurium in a variety of proportions), or even liquid.

As used herein, the term “biological material” has the same meaning of “biomaterial” as understood by a person skilled in the art. Without limiting its meaning, biological materials or biomaterials can generally be produced either in nature or synthesized in the laboratory using a variety of chemical approaches utilizing organic compounds (e.g., small organic molecules or polymers) or inorganic compounds (e.g., metallic components or ceramics). They generally can be used or adapted for a medical application, and thus comprise whole or part of a living structure or biomedical device which performs, augments, or replaces a natural function. Such functions may be benign, like being used for a heart valve, or may be bioactive with a more interactive functionality such as hydroxyl-apatite coated hip implants. Biomaterials can also be used every day in dental applications, surgery, and drug delivery. For instance, a construct with impregnated pharmaceutical products can be placed into the body, which permits the prolonged release of a drug over an extended period of time. A biomaterial may also be an autograft, allograft, or xenograft which can be used as a transplant material. All these materials that have found applications in other medical or biomedical fields can also be used in the present invention.

As used herein, the term “microelectronic technology or process” generally encompasses the technologies or processes used for fabricating micro-electronic and optical-electronic components. Examples include lithography, etching (e.g., wet etching, dry etching, or vapor etching), oxidation, diffusion, implantation, annealing, film deposition, cleaning, direct-writing, polishing, planarization (e.g., by chemical mechanical polishing), epitaxial growth, metallization, process integration, simulation, or any combinations thereof. Additional descriptions on microelectronic technologies or processes can be found in, e.g., Jaeger, Introduction to Microelectronic Fabrication, 2^{nd} Ed., Prentice Hall, 2002; Ralph E. Williams, Modern GaAs Processing Methods, 2^{nd} Ed., Artech House, 1990; Robert F. Pierret, Advanced Semiconductor Fundamentals, 2^{nd} Ed., Prentice Hall, 2002; S. Campbell, The Science and Engineering of Microelectronic Fabrication, 2^{nd} Ed., Oxford University Press, 2001, the contents of all of which are incorporated herein by reference in their entireties.

As used herein, the term “selective” as included in, e.g., “patterning material B using a microelectronics process selective to material A”, means that the microelectronics process is effective on material B but not on material A, or is substantially more effective on material B than on material B (e.g., resulting in a much higher removal rate on material B than on material A and thus removing much more material B than material A).

As used herein, the term “carbon nano-tube” generally refers to as allotropes of carbon with a cylindrical nanostructure. See, e.g., Carbon Nanotube Science, by P. J. F. Harris, Cambridge University Press, 2009, for more details about carbon nano-tubes.

Through the use of a single micro-device or a combination of micro-devices integrated into a disease detection apparatus, the disease detection capabilities can be significantly improved in terms of sensitivity, specificity, speed, cost, apparatus size, functionality, and ease of use, along with reduced invasiveness and side-effects. A large number of micro-device types capable of measuring a wide range of microscopic properties of biological sample for disease detection can be integrated and fabricated into a single detection apparatus using micro-fabrication technologies and novel process flows disclosed herein. While for the purposes of demonstration and illustration, a few novel, detailed examples have been shown herein on how microelectronics or nano-fabrication techniques and associated process flows can be utilized to fabricate highly sensitive, multi-functional, and miniaturized detection devices, the principle and general approaches of employing microelectronics and nano-fabrication technologies in the design and fabrication of high performance detection devices have been contemplated and taught, which can and should be expanded to various combination of fabrication processes including but not limited to thin film deposition, patterning (lithography and etch), planarization (including chemical mechanical polishing), ion implantation, diffusion, cleaning, various materials, and various process sequences and flows and combinations thereof.

### BRIEF DESCRIPTIONS OF THE FIGURES

FIG. 1 (a) illustrates a set of traditional detection apparatus each of which detects and relies on a single detection technology. FIG. 1 (b) and FIG. (c) are illustration of a detection apparatus of this invention where multiple sub-equipment units are integrated.

FIG. 2 is a schematic illustration of a detection apparatus of this invention which comprises multiple sub-equipment units, a delivery system, and a central control system.

FIG. 3 is a perspective illustration of a detection apparatus of this invention in which a biological sample placed in it or moving through it can be tested.

FIG. 4 illustrates an apparatus of the present invention which comprises two slabs each of which is fabricated with one or more detection or probing units.

FIG. 5 is a perspective, cross-sectional illustration of a detection apparatus of this invention with multiple micro-devices placed at a desired distance for time of flight measurements with enhanced sensitivity, specificity, and speed, including time dependent or dynamic information.

FIG. 6 is a perspective illustration of a novel set of microscopic probes, included in a detection apparatus of this invention, for detecting various electronic or magnetic states, configurations, or other properties of a biological sample (e.g., a cell).

FIG. 7 is a perspective illustration of a novel four-point probe, included in a detection apparatus of this invention, for detecting weak electronic signal in a biological sample (e.g., a cell).

FIG. 8 illustrates a fluid delivery system, which is a pretreatment part for the detection apparatus, and it delivers a sample or auxiliary material at a desired pressure and speed into a device.

FIG. 9 illustrates how a micro-device in a disease detection apparatus of this invention can communicate, probe, detect, and optionally treat and modify biological subjects at a microscopic level.

FIG. 10 illustrates another micro-device or sub-equipment that can detect the optical properties of the biological subject with a set of optical sensors.

FIG. 11 illustrates another micro-device or sub-equipment that can separate biological subjects of different geometric size and detect their properties respectively.

FIG. 12 illustrates a micro-device or sub-equipment that can measure the acoustic property of a biological subject.

FIG. 13 illustrates a micro-device or sub-equipment that can measure the internal pressure of a biological subject.

FIG. 14 illustrates a micro-device or sub-equipment that has concaves between the probe couples, in the bottom or ceiling of the channel.

FIG. 15 illustrates another micro-device or sub-equipment that has concaves of a different shape from those illustrated in FIG. 14.

FIG. 16 illustrates a micro-device or sub-equipment that has a stepped channel.

FIG. 17 illustrates a micro-device or sub-equipment that has a set of thermal meters.

FIG. 18 illustrates a micro-device or sub-equipment that includes a carbon nano-tube as the channel with DNA contained therein.

FIG. 19 illustrates a micro-device or sub-equipment that includes a detecting device and an optical sensor.

FIG. 20 illustrates an integrated apparatus of this invention that includes a detecting device and a logic circuitry.

FIG. 21 illustrated a micro-device or sub-equipment that includes a detecting device and a filter.

FIG. 22 illustrates how apparatus of this invention can be used to measure a DNA′ geometric factors.

FIG. 23 illustrates an apparatus of this invention with a cover atop the trench to form a channel.

FIG. 24 is a diagram of sub-equipment unit for detecting a disease in a biological subject.

FIG. 25 shows an example of a sample filtration unit.

FIG. 26 shows another example of a sample filtration unit.

FIG. 27 is a diagram of a pre-processing unit of an apparatus of this invention.

FIG. 28 is a diagram of an information processing unit of an apparatus of this invention.

FIG. 29 shows the integration of multiple signals which results in cancellation of noise and enhancement of signal to noise ratio.

FIG. 30 shows a novel disease detection method in which at least one probe object is launched at a desired speed and direction toward a biological subject, resulting in a collision.

FIG. 31 shows a process of this invention for detecting a biological subject using disease detection apparatus.

FIG. 32 shows another embodiment of disease detection process wherein diseased and healthy biological subjects are separated and the diseased biological subjects are delivered to further test.

FIG. 33 shows an arrayed biological detecting device wherein a series of detecting devices fabricated into an apparatus.

FIG. 34 shows another embodiment of a disease detection device of the current invention including inlet and outlet of the device, the channel where the biological subject passes through, and detection devices aligned along the walls of the channel.

FIG. 35 shows an example of the apparatus of this invention packaged and ready for use.

FIG. 36 shows another example of the apparatus of this invention that is packaged and ready for use.

FIG. 37 shows yet another example of the apparatus of this invention that is packaged and ready for use.

FIG. 38 shows an apparatus of this invention that has a channel (trench) and an array of micro sensors.

FIG. 39 shows another apparatus of this invention comprising several “sub-devices.”

FIG. 40 shows an example of the apparatus of this invention which includes an application specific integrated circuit (ASIC) chip with I/O pads.

FIG. 41 is a diagram of the underlying principal of the apparatus of this invention which functions by combining various pre-screening and detection methods in unobvious ways.

FIG. 42 shows cross-sectional and outside views of a channel into which a biological subject can flow.

FIG. 43 shows a biological subject to be detected passing through a channel aligned with detectors along its passage in an apparatus of this invention.

FIG. 44 is a view of the apparatus of this invention showing one or two sorting units therein.

FIG. 45 shows an apparatus of this invention with a high number of desired structures fabricated simultaneously on the same chip.

FIG. 46 shows another novel device layout for sorting, screening, separating, probing and detecting diseased biological entities, in which a desired component or multiple components through the middle channel into the middle chamber can play a wide range of roles.

FIG. 47 shows that, compared with multiple stand-alone detection apparatuses, an apparatus of this invention with multiple sub-units of different functions and technologies assembled or integrated has a significantly reduced apparatus volume or size, therefore reduced costs since many common hardware (e.g., a sample handling unit, a sample measurement unit, a data analysis unit, a display, a printer, etc.) can be shared in an integrated apparatus.

FIG. 48 shows that when multiple sub-units with different functions and technologies are assembled into one apparatus, a more diverse functionality, improved detection functionality, sensitivity, detection versatility, and reduced volume and cost can be achieved, where a number of common utilities including, e.g., input hardware, output hardware, sample handling unit, sample measurement unit, data analysis unit and data display unit can be shared.

FIG. 49 shows a number of different classifications of biological information are collected in a device and processed in the novel technology.

FIG. 50 shows measured information in this novel technology includes protein, cellular and molecular level information, or combination of them.

FIG. 51 shows signals from different biological classifications may interact, combine, and/or amplify to enhance signal in this novel technology.

FIG. 52 shows detected signal in this novel technology as a function of cancer cell concentration. Signal increases with increasing amount of cancer cells.

FIG. 53 shows detected signal in this novel technology as a function of a bio-marker level. Signal increases with increasing level of bio-marker.

FIG. 54 shows Advantage of this novel technology compared with traditional bio-marker (AFP) for liver cancer. Using 58 confirmed liver cancer samples, sensitivity of this novel technology is 79.3%, while that of AFP is 55.9%.

FIG. 55 shows the results of detected signal CDA before and after adding molecular level reaction triggering agent.

FIG. 56 shows the numbers of actual samples tested by this invention and the unexpected results achieved or shown by these tests.

FIG. 57 shows the results of a multi-level detection system of this invention.

FIG. 58 shows the CDA values of the control group, non-cancer disease group and cancer group.

FIG. 59 shows the relationship between disease state and detected cell signaling properties and/or cell media properties.

FIG. 60 shows a scheme of cells, proteins, and genetic components (DNA, RNA, etc.) and their surrounding liquid media (e.g., blood).

FIG. 61 shows scanning curves of control (healthy) and lung cancer cell lines.

FIG. 62 shows a typical scanning curve for control (healthy) whole blood sample.

FIG. 63 shows scanning curves for control (healthy) whole blood sample and liver cancer whole blood sample.

FIG. 64 shows scanning curves for control (healthy), disease, and liver cancer whole blood samples.

FIG. 65 shows comparison of claimed technology in this application versus circulating tumor cell (CTC) and circulating tumor (cancer) DNA (ct-DNA). In this technology, signal exists for all groups starting with healthy group and rises rapidly with disease group, pre-cancer group and cancer group, with a high signal to noise ratio (schematically each dot represents signal, the higher the signal, the more dots), while CTC and ct-DNA technologies only have signal in cancer stage II, with a very weak signal, and expected poor signal to noise ratio).

FIG. 66 shows that CDA technology is a multi-level and multi-parameter test that can also be carried out in conjunction with other tests including bio-markers (protein level), CTC (cellular level), and/or ct-DNA and other DNA based tests (genetic tests).

FIG. 67 shows a schematic of a proposed model, in which shift in bio-physical properties such as electrical properties cause changes at cellular, protein, and molecular (gene) levels which result in changes at immunity and inflammation, and likelihood (or less likelihood) of diseases and cancer occurrence.

FIG. 68 shows that as CDA increases and electrical current, conductance, ion level, membrane potential and polarization decrease, a number of cellular level (cell signaling, cell repulsion, resting potential and cell surface charge decrease) and molecular level (DNA surface charge decrease, quantum mechanical effect change, and DNA mutation increases) properties degrade, resulting in increased disease and cancer occurrence.

FIG. 69 shows the CDA value (a value based on the measured properties claimed in this patent application and after data analysis) for control (healthy) group, non-cancer disease group, and cancer group. The DCA value becomes progressively higher from healthy stage, to non-cancer disease group, and to cancer group.

FIG. 70 shows that as electrical current and conductance decrease (ion (e.g., potassium, chloride, sodium, and calcium) concentration or net ion concentration or charge decreases), a number of cellular level (cell signaling, cell repulsion, resting potential, membrane potential and cell surface charge decrease) properties change and degrade.

FIG. 71 shows the changes in electrical properties of DNA surrounding media and/or DNA surface charge between health and cancer cases.

FIG. 72 shows that the CDA technology has higher sensitivity and specificity than traditional CT imaging.

FIG. 73 shows that the CDA values appear to correlate with mutation frequency for (a) healthy, (b) lung cancer just after diagnosis and before surgery, and (c) after surgery and treatment individuals/groups.

FIG. 74 shows use of the CDA technology for prognosis of a targeted drug treatment of small cell lung cancer at three stages, i.e., after diagnosis, after phase 1 treatment, and after phase 2 treatment.

FIG. 75 shows a schematic of cell membranes with intracellular and extracellular regions, with decreasing membrane potential and net charge Q in extracellular region.

FIG. 76 shows a schematic of membranes of two cells showing membrane potential, intracellular space, and extracellular space.

FIG. 77 shows a schematic of an apparatus of this invention for treating a disease, using physical or bio-physical approaches.

FIG. 78 shows a schematic of another apparatus of this invention for treating a disease, using physical or bio-physical approaches.

FIG. 79 illustrate the change of the property of the biological subject after the disease treatment of this invention.

FIGS. 80A-80O illustrate an apparatus for treating a disease, with a coil surrounding channel that is capable of applying an energy onto the biological subject.

### DETAILED DESCRIPTION OF THE INVENTION

While existing cancer screening tests and treating methods lack the ability to effectively detect and/or impact multiple types of cancer at the same time (e.g., in one test), the present invention provides a novel technology that can simultaneously detect (or even treat) a significantly increased number of diseases—including cancers or pre-cancer diseases (e.g., over 20 types of cancer)—with higher speed, higher sensitivity and specificity (75%˜90% on over 20 types of cancer), simpler process, reduced costs, and no side effects. Compared to conventional technologies, the novel cancer treatment technology of this invention has a number of major, unexpected advantages—including, e.g., lower costs, far less side efforts, easier recovery, capability of cancer prevention, improved survival rate and ease of use. The cancer treatment of this invention can use a low dosage and/or a weak field and/or energy for treatment.

One aspect of the present invention relates to apparatus for detecting a disease in a biological subject in vivo or in vitro (e.g., human being, an organ, a tissue, or cells in a culture). Each apparatus comprises a delivery system, at least two sub-equipment units, and optionally a central control system. Each sub-equipment is capable of measuring at least a microscopic property of a biological sample. Accordingly, the apparatus of this invention can detect different parameters of the biological subject and provide accuracy, sensitivity, specificity, efficiency, non-invasiveness, practicality, conclusive, and speed in early-stage disease detection at reduced costs. In addition, the apparatus of this invention has some major advantages, such as reducing effective foot print (e.g., defined as function per unit space), reducing space for the medical devices, reducing overall cost, and providing conclusive and effective diagnosis by one device.

The delivery system can be a fluid delivery system. By the constant pressure fluid delivery system, microscopic biological subjects can be delivered onto or into one or more desired sub-equipment units of the apparatus.

As a key component of the apparatus, the micro-device should include means to perform at least the function of addressing, controlling, forcing, receiving, amplifying, or storing information from each probing address. As an example, the apparatus can further include a central control system for controlling the biological subject matter to be transported to one or more desired sub-equipment units and reading and analyzing a detected data from each sub-equipment unit. The central control system includes a controlling circuitry, an addressing unit, an amplifier circuitry, a logic processing circuitry, a memory unit, an application specific chip, a signal transmitter, a signal receiver, or a sensor.

In some embodiments, the fluid delivering system comprises a pressure generator, a pressure regulator, a throttle valve, a pressure gauge, and distributing kits. As examples of these embodiments, the pressure generator can include a motor piston system and a bin containing compressed gas; the pressure regulator (which can consist of multiple regulators) can down-regulate or up-regulate the pressure to a desired value; the pressure gauge feeds back the measured value to the throttle valve which then regulates the pressure to approach the target value.

The biological fluid to be delivered can be a sample of a biological entity to be detected for disease or something not necessarily to be detected for disease. In some embodiments, the fluid to be delivered is liquid (e.g., a blood sample or a lymph sample). The pressure regulator can be a single pressure regulator or multiple pressure regulators which are placed in succession to either down-regulate or up-regulate the pressure to a desired level, particularly when the initial pressure is either too high or too low for a single regulator to adjust to the desired level or a level that is acceptable for an end device or target.

Optionally, the apparatus includes additional features and structures to deliver a second liquid solution containing at least an enzyme, protein, oxidant, reducing agent, catalyst, radio-active component, optical emitting component, or ionic component. This second liquid solution can be added to the sample to be measured before or during sorting of the biological subject sample to be measured, or before or during the measurement (i.e., detection) of the biological subject sample, for the purposes of further enhancing the apparatus' measurement sensitivity.

In some other embodiments, the system controller includes a pre-amplifier, a lock-in amplifier, an electrical meter, a thermal meter, a switching matrix, a system bus, a nonvolatile storage device, a random access memory, a processor, or a user interface. The interface can include a sensor which can be a thermal sensor, a flow meter, an optical sensor, an acoustic detector, a current meter, an electrical sensor, a magnetic sensor, an electro-magnetic sensor, a pH meter, a hardness measurement sensor, an imaging device, a camera, a piezo-electrical sensor, a piezo-photronic sensor, a piezo-electro photronic sensor, an electro-optical sensor, an electro-thermal sensor, a bio-electrical sensor, a bio-marker sensor, a bio-chemical sensor, a chemical sensor, an ion emission sensor, a photo-detector, an x-ray sensor, a radiation material sensor, an electrical sensor, a voltage meter, a thermal sensor, a flow meter, or a piezo- meter.

In still some other embodiments, apparatus of this invention further includes a biological interface, a system controller, a system for reclaiming or treatment medical waste. The reclaiming and treatment of medical waste can be performed by the same system or two different systems.

Another aspect of this invention provides apparatus for interacting with a cell, which include a device for sending a signal to the cell and optionally receiving a response to the signal from the cell.

In some embodiments, the interaction with the cell can be probing, detecting, sorting, communicating with, treating, or modifying with a coded signal that can be a thermal, optical, acoustical, biological, chemical, electro-mechanical, electro-chemical, electro-optical, bio-electro-optical, bio-thermal optical, electro-chemical optical, electro-chemical-mechanical, bio-chemical, bio-mechanical, bio-electro-mechanical, bio-electro-chemical, bio-electro-chemical-mechanical, electric, magnetic, electro-magnetic, physical, or mechanical signal, or a combination thereof.

In some other embodiments, the device or the sub-equipment unit contained in the apparatus can include multiple surfaces coated with one or more elements or combinations of elements, and a control system for releasing the elements. In some instances, the control system can cause release of the elements from the device surface via an energy including but not limited to thermal energy, mechanical energy, gravitational field energy, quantum mechanical energy, optical energy, acoustic energy, electrical energy, electro-magnetic energy, magnetic energy, radiation energy, or mechanical energy in a controlled manner. The energy can be in the pulsed form at desired frequencies.

In some other embodiments, the device or the sub-equipment unit contained in the apparatus includes a first component for storing or releasing one element or a combination of elements onto the surface of the cell or into the cell; and a second component for controlling the release of the elements (e.g., a circuitry for controlling the release of the elements). The elements can be a biological component, a chemical compound, ions, catalysts, Ca, C, Cl, Co, Cu, H, I, Fe, Mg, Mn, N, O, P, F, K, Na, S, Zn, or a combination thereof. The signal, pulsed or constant, can be in the form of a released element or combination of elements, and it can be carried in a liquid solution, gas, or a combination thereof. In some instances, the signal can be at a frequency ranging from about 1×10⁻⁴ Hz to about 100 MHz or ranging from about 1×10⁻⁴ Hz to about 10 Hz, or at an oscillation concentration ranging from about 1.0 nmol/L to about 10.0 mmol/L. Also, the signal comprises the oscillation of a biological component, a chemical compound, Ca, C, Cl, Co, Cu, H, I, Fe, Mg, Mn, N, O, P, F, K, Na, S, Zn, or a combination thereof, e.g., at desired oscillating frequencies.

In some embodiments, the signal to be sent to the cell can be in the form of oscillating element, compound, or an oscillating density of a biological component, and a response to the signal from the cell is in the form of oscillating element, compound, or an oscillating density of a biological component.

In some embodiments, the device or the sub-equipment unit can be coated with a biological film, e.g., to enhance compatibility between the device and the cell.

In some other embodiments, the device or the sub-equipment unit can include components for generating a signal to be sent to the cell, receiving a response to the signal from the cell, analyzing the response, processing the response, and interfacing between the device and the cell.

Still another aspect of this invention provides devices or sub-equipment units each including a micro-filter, a shutter, a cell counter, a selector, a micro-surgical kit, a timer, and a data processing circuitry. The micro-filter can discriminate abnormal cells by a physical property (e.g., dimension, shape, or velocity), mechanical property, electric property, magnetic property, electro-magnetic, thermal property (e.g., temperature), optical property, acoustical property, biological property, chemical property, electro-chemical property, bio-chemical property, bio-electro-chemical property, bio-electro-mechanical property, or electro-mechanical property. The devices each can also include one or more micro-filters. Each of these micro-filters can be integrated with two cell counters, one of which is installed at the entrance of each filter well, while the other is installed at the exit of each filter well. The shape of the micro-filter's well is rectangle, ellipse, circle, or polygon; and the micro-filter's dimension ranges from about 0.1 μm to about 500 μm or from about 5 um to about 200 um. As used herein, the term “dimension” means the physical or feature size of the filter opening, e.g., diameter, length, width, or height. The filter can be coated with a biological or bio-compatible film, e.g., to enhance compatibility between the device and the cell.

In addition to separation of biological entity by its size and other physical features, the filter can also contain additional features and functions to perform biological entity separation via other properties, which comprise of mechanical property, electric property, magnetic property, electro-magnetic, thermal property (e.g., temperature), optical property, acoustical property, biological property, chemical property, electro-chemical property, bio-chemical property, bio-electro-chemical property, bio-electro-mechanical property, and electro-mechanical property.

In some embodiments of these devices, the shutter sandwiched by two filter membranes can be controlled by a timer (thus time shutter). The timer can be triggered by the cell counter. For instance, when a cell passes through the cell counter of the filter entrance, the clock is triggered to reset the shutter to default position, and moves at a preset speed towards the cell pathway, and the timer records the time as the cell passes through the cell counter at the exit.

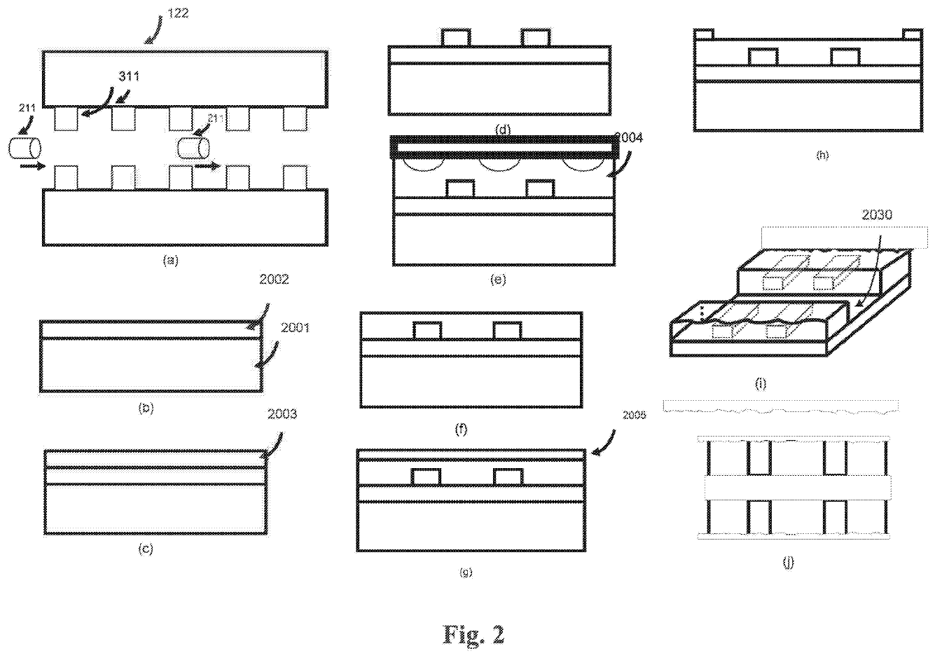

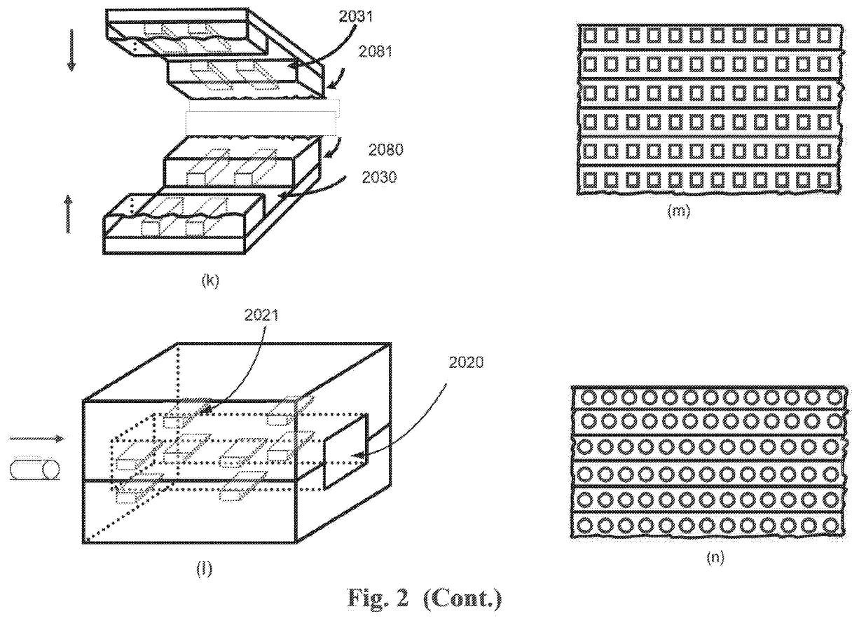

Still a further aspect of this invention provides methods for fabricating a micro-device with micro-trench and probe embedded in the micro-trench's sidewalls. A micro-trench is an unclosed tunnel (see, e.g., FIG. 2(i), 2030), which can be coupled with another upended symmetric trench (see, e.g., FIG. 2(k), 2031) to form a closed channel (see, e.g., FIG. 2(l), 2020). The method may include chemical vapor deposition, physical vapor deposition, or atomic layer deposition to deposit various materials on a substrate (where the substrate can be a semiconductor material such as silicon, or an insulating material such as glass or silicon dioxide material); patterning the deposited layer(s) utilizing methods comprising of lithography, etch, and chemical mechanical polishing to form desired features (such as a trench); chemical mechanical planarization for surface planarization; chemical cleaning for particle removal; diffusion or ion implantation for doping elements into specific layers; or thermal anneal to reduce the crystal defects and activate diffused ions. An example of such method includes: depositing a first material onto a substrate; depositing a second material onto the first material and patterning the second material by a microelectronic process (e.g., lithography, etch) to form a detecting tip; depositing a third material on the second material and then planarize the third material by a polishing process; depositing a fourth material on the third material and patterning the fourth material first by a microelectronic process (e.g., lithography, etch) and then by a microelectronic process (e.g., another etch) to remove a portion of the third material and optionally a portion of the first material while this etch is typically selective to the second material (lower etch rate for the second material), in which the fourth material serves as a hardmask. A hardmask generally refers to a material (e.g., inorganic dielectric or metallic compound) used in semiconductor processing as an etch mask in lieu of polymer or other organic “soft” materials. In one embodiment, a channel is formed in the substrate layer (such as a silicon or a silicon dioxide or glass layer) or in the layer(s) above the substrate layer, with at least one probe (such as gold, tungsten, aluminum, silver, copper, or nickel conductive probing tip) being formed on the wall of the channel to probe desired biological sample properties (such as physical, bio-physical, or bio-chemical properties).

In some embodiments, the method further includes coupling two devices or sub-equipment units that are thus fabricated and symmetric (i.e., a flipped mirror) to form a detecting device with channels. The entrance of each channel can be optionally bell-mouthed, e.g., such that the size of channel's opening end (the entrance) is larger than the channel's body, thereby making it easier for a cell to enter the channel. The shape of each channel's cross-section can be rectangle, ellipse, circle, or polygon. The micro-trenches of the coupled two micro-devices can be aligned by the module of alignment marks designed on the layout of the micro-device. The dimension of the micro-trench can range from about 0.1 um to about 500 um.

Alternatively, the method can also include covering the micro-trench of the micro-device with a flat panel. Such a panel can comprise or be made with silicon, SiGe, SiO₂, Al₂O₃, quartz, low optical loss glasses, or other optical materials. Examples of other potentially suitable optical materials include acrylate polymer, AgInSbTe, synthetic alexandrite, arsenic triselenide, arsenic trisulfide, barium fluoride, CR-39, cadmium selenide, caesium cadmium chloride, calcite, calcium fluoride, chalcogenide glass, gallium phosphide, GeSbTe, germanium, germanium dioxide, glass code, hydrogen silsesquioxane, Iceland spar, liquid crystal, lithium fluoride, lumicera, METATOY, magnesium fluoride, agnesium oxide, negative index metamaterials, neutron super-mirror, phosphor, picarin, poly(methyl methacrylate), polycarbonate, potassium bromide, sapphire, scotophor, spectralon, speculum metal, split-ring resonator, strontium fluoride, yttrium aluminum garnet, yttrium lithium fluoride, yttrium orthovanadate, ZBLAN, zinc selenide, and zinc sulfide.

In other embodiments, the method can further include integrating three or more sub-equipment units or devices thus fabricated to yield an enhanced device with an array of the channels.

Another aspect of this invention relates to a set of novel process flows for fabricating micro-devices (including micro-probes and micro-indentation probes) for their applications in disease detection by measuring microscopic properties of a biological sample. The micro-devices can be integrated into detection apparatus of this invention as sub-equipment units to measure one or more properties at microscopic levels. For example, a cancerous cell may have a different hardness (harder), density (denser), and elasticity than a normal cell.

Another aspect of this invention is to involve in cellular communications and regulate cellular decision or response (such as differentiation, dedifferentiation, cell division and cell death) with fabricated signals generated by the micro-devices disclosed herein. This could be further employed to detect and treat diseases.

Another aspect of the current application is that the inventive method or measured parameter in the method is a function of at least two levels F (level 1, level 2), where level 1 can be a biological entity such as protein and level 2 can be another biological entity such as genetics, where the measured signal strength of F (level 1, level 2) is greater than the sum of the signal containing only level 1 information f (level 1) and the signal containing only level 2 information f (level 2):

Signal strength of F (level 1, level 2)>signal strength of f (level 1)+signal strength of f (level 2)

The above novel feature and property can be extended to a measured parameter which is a function containing many levels F (level 1, level 2, level 3 level n). One novel and unobvious feature of this innovation is that the measured signal in a parameter containing multiple biological levels is synergistically enhanced over the measured signals with each signal containing a single biological level only. With this approach, the typically weak detection signal in disease detection such as cancer detection (especially in early stage cancer detection) can be effectively enhanced or magnified, making early disease detection possible and more effective.

To further enhance measurement capabilities, multiple micro-devices can be implemented into a piece of detection apparatus as sub-equipment units employing the time of flight technique, in which at least one probing micro-device and one sensing micro-device placed at a preset, known distance. The probing micro-device can apply a signal (e.g., a voltage, a charge, an electrical field, a laser beam, a thermal pulse, a train of ions, or an acoustic wave) to the biological sample to be measured, and the detection (sensing) micro-device can measure response from or of the biological sample after the sample has traveled a known distance and a desired period of time. For instance, a probing micro-device can apply an electrical charge to a cell first, and then a detection (sensing) micro-device subsequently measures the surface charge after a desired period of time (T) has lapsed and the cell has traveled a certain distance (L).

The micro-devices or the sub-equipment units contained in the apparatus of this invention can have a wide range of designs, structures, functionalities, flexibilities, and applications due to their diverse properties, high degree of flexibilities, and ability of integration, miniaturization, and manufacturing scalability. They include, e.g., a voltage comparator, a four point probe, a calculator, a logic circuitry, a memory unit, a micro cutter, a micro hammer, a micro shield, a micro dye, a micro pin, a micro knife, a micro needle, a micro thread holder, micro tweezers, a micro laser, a micro optical absorber, a micro mirror, a micro wheeler, a micro filter, a micro chopper, a micro shredder, micro pumps, a micro absorber, a micro signal detector, a micro driller, a micro sucker, a micro tester, a micro container, a signal transmitter, a signal generator, a friction sensor, an electrical charge sensor, a temperature sensor, a hardness detector, an acoustic wave generator, an optical wave generator, a heat generator, a micro refrigerator and a charge generator.

Further, it should be noted that advancements in manufacturing technologies have now made fabrications of a wide range of micro-devices and integration of various functions onto the same device highly feasible and cost effective. The typical human cell size is about 10 microns. Using state-of-the-art integrated circuit fabrication techniques, the minimum feature size defined on a micro-device can be as small as 0.1 micron or below. Thus, it is ideal to utilize the disclosed micro-devices for biological applications.

In terms of materials for the micro-devices in the apparatus of this invention, the general principle or consideration is the material's compatibility with a biological entity. Since the time in which a micro-device is in contact with a biological sample (e.g., a cell) may vary, depending on its intended application, a different material or a different combination of materials may be used to make the micro-device. In some special cases, the materials may dissolve in a given pH in a controlled manner and thus may be selected as an appropriate material. Other considerations include cost, simplicity, ease of use and practicality. With the significant advancements in micro fabrication technologies such as integrated circuit manufacturing technology, highly integrated devices with minimum feature size as small as 0.1 micron can now be made cost-effectively and commercially. One good example is the design and fabrication of micro electro mechanical devices (MEMS), which now are being used in a wide variety of applications in the electronics industry and beyond.

Good disease (cancer and non-cancer) detection results in terms of measurement sensitivity and specificity have been obtained on multiple types of cancer tested, demonstrating validity of the apparatus of this invention for improved ability to detect diseases (e.g., cancers), particularly in their early stages. The present invention provides novel “Cancer Differentiation Analysis” (CDA) liquid biopsy technology. The experimental results have also shown that multiple cancer types can be detected using the disclosed apparatus, which itself is an improvement over many existing detection apparatuses.

Specifically, studies utilizing the apparatus of this invention have been carried out on multiple types of cancer and non-cancer diseases (including an inflammatory disease, diabetes, a lung disease, a heart disease, a liver disease, a gastric disease, a biliary disease, or a cardiovascular disease). In these studies, whole blood samples were used within 5 days after being obtained and/or properly transported/stored in a 0.5-20° C. refrigerated environment. The samples of the control group were obtained from healthy people confirmed by physical examinations with normal AFP and CEA values (in normal ranges).

TABLE 1
Data from the Test for Lung Diseases

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA | Control | 981 | 54 | 22-91 | 59 | 61 | 36.55 | 36.20 | 7.18 |
| | Lung | 95 | 71 | 21-90 | 65 | 67 | 45.75 | 45.66 | 22.67 |
| | Disease | | | | | | | | |
| | Pulmonary | 75 | 67 | 21-85 | 65 | 66 | 45.78 | 45.83 | 9.08 |
| | infection | | | | | | | | |
| | Pneumonia | 14 | 79 | 22-87 | 61 | 63 | 44.49 | 45.25 | 9.21 |
| | Chronic | 4 | 100 | 73-90 | 81 | 81 | 45.63 | 43.55 | 6.56 |
| | obstructive | | | | | | | | |
| | pulmonary | | | | | | | | |
| | disease | | | | | | | | |
| | Tuberculosis | 2 | 100 | 65-66 | 66 | 66 | 53.87 | 53.87 | 11.92 |

TABLE 2
Data from Tests for Diabetes

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA (rel. | Control | 981 | 54 | 22-91 | 59 | 61 | 36.55 | 36.20 | 7.18 |
| units) | Diabetes | 62 | 55 | 37-86 | 62 | 62 | 44.31 | 45.01 | 12.47 |
| | Type-2 | 39 | 49 | 37-86 | 61 | 62 | 47.08 | 46.45 | 13.34 |
| | Diabetes | | | | | | | | |
| | Unclear | 23 | 65 | 43-86 | 63 | 62 | 39.62 | 41.92 | 9.32 |
| | types | | | | | | | | |

TABLE 3
Data from Tests for Heart Diseases

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA (rel. | Control | 981 | 54 | 22-91 | 59 | 61 | 36.55 | 36.20 | 7.18 |
| units) | Heart | 54 | 45 | 21-105 | 73 | 75 | 44.24 | 44.43 | 11.97 |
| | Disease | | | | | | | | |
| | Coronary | 26 | 38 | 50-94 | 71 | 70 | 41.99 | 42.70 | 13.39 |
| | disease | | | | | | | | |
| | Other | 14 | 57 | 61-91 | 76 | 76 | 46.88 | 47.73 | 6.86 |
| | heart | | | | | | | | |
| | disease | | | | | | | | |
| | Heart failure | 9 | 44 | 74-105 | 82 | 80 | 48.60 | 45.41 | 14.58 |
| | Arrhythmia | 5 | 20 | 21-85 | 62 | 70 | 40.69 | 44.18 | 9.11 |

TABLE 4
Data from Tests for Liver Diseases

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA (rel. | Control | 981 | 54 | 22-91 | 59 | 61 | 36.55 | 36.20 | 7.18 |
| units) | Liver | 160 | 68 | 24-87 | 55.56 | 53.50 | 44.29 | 44.75 | 8.32 |
| | Disease | | | | | | | | |
| | Cirrhosis | 88 | 78 | 30-87 | 57.68 | 55.00 | 43.68 | 43.72 | 8.62 |
| | Hepatitis | 56 | 63 | 24-76 | 54.27 | 52.50 | 43.32 | 43.84 | 7.74 |

TABLE 5
Data from Tests for Gastric Diseases

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA (rel. | Control | 981 | 54 | 22-91 | 59 | 61 | 36.55 | 36.20 | 7.18 |
| units) | Gastric | 47 | 60 | 29-89 | 60.81 | 63.00 | 44.24 | 44.90 | 9.29 |
| | Disease | | | | | | | | |
| | Gastritis | 28 | 61 | 29-89 | 60.29 | 62.00 | 45.16 | 45.01 | 9.37 |
| | Gastric | 12 | 67 | 33-71 | 61.00 | 66.00 | 41.70 | 44.37 | 8.17 |
| | polyp | | | | | | | | |
| | Gastric | 2 | 50 | 59-79 | 69.00 | 69.00 | 36.76 | 36.76 | 11.12 |
| | ulcer | | | | | | | | |

TABLE 6
Summary of Descriptive Statistics

| | | | | | | | CDA | CDA | CDA |
|---|---|---|---|---|---|---|---|---|---|
| | | | Gender | Age | Age | Age | Mean | Median | STDEV |
| Group | | Samples | (Male %) | Range | Mean | Median | (rel. units) | (rel. units) | (rel. units) |
| CDA (rel. | Control | 981 | 54 | 22-91 | 59 | 61 | 36.55 | 36.20 | 7.18 |
| units) | Lung | 95 | 71 | 21-90 | 65 | 67 | 45.75 | 45.66 | 22.67 |
| | Disease | | | | | | | | |
| | Diabetes | 62 | 55 | 37-86 | 62 | 62 | 44.31 | 45.01 | 12.47 |
| | Heart | 54 | 45 | 21-105 | 73 | 75 | 44.24 | 44.43 | 11.97 |
| | Disease | | | | | | | | |
| | Liver | 160 | 68 | 24-87 | 55.56 | 53.50 | 44.29 | 44.75 | 8.32 |
| | Disease | | | | | | | | |
| | Gastric | 47 | 60 | 29-89 | 60.81 | 63.00 | 44.24 | 44.90 | 9.29 |
| | Disease | | | | | | | | |
| | Biliary | 28 | 57 | 21-85 | 60.11 | 60.50 | 45.75 | 46.57 | 11.82 |
| | Disease | | | | | | | | |

TABLE 7
Results of ROC Curve Analysis

| | Area Under | | | |
|---|---|---|---|---|
| | the Curve | Cut-off Value | | |
| Group | (rel. units) | (rel. units) | Sensitivity | Specificity |
| Lung Disease | 0.788 | 41 | 74.7% | 73.9% |
| Diabetes | 0.727 | 41 | 72.6% | 72.3% |
| Heart Disease | 0.736 | 41 | 74.1% | 74.3% |
| Liver Disease | 0.758 | 41 | 70.0% | 73.8% |
| Gastric Disease | 0.740 | 41 | 74.5% | 74.3% |
| Biliary Disease | 0.779 | 41 | 82.1% | 74.4% |

CDA value is obtained from an algorithm using calculation based on tested values from the studies. CDA value increases with risks of diseases. In other words, the higher the CDA values, the higher the risks of diseases.

As the above tables show, the CDA values are higher for various diseases (mid 40s) than those of control (healthy) group (around 36). Statistical analysis of CDA values for those two groups shows that there was a statistically significant difference in CDA values between those two groups. Accordingly, the studies above show that the apparatus and methods of this invention were able to distinguish some major diseases from control group, with sensitivity and specificity likely higher than existing technologies.

Set forth below are several illustrations or examples of apparatus of this invention containing a class of innovative micro-devices that are integrated as sub-equipment units.

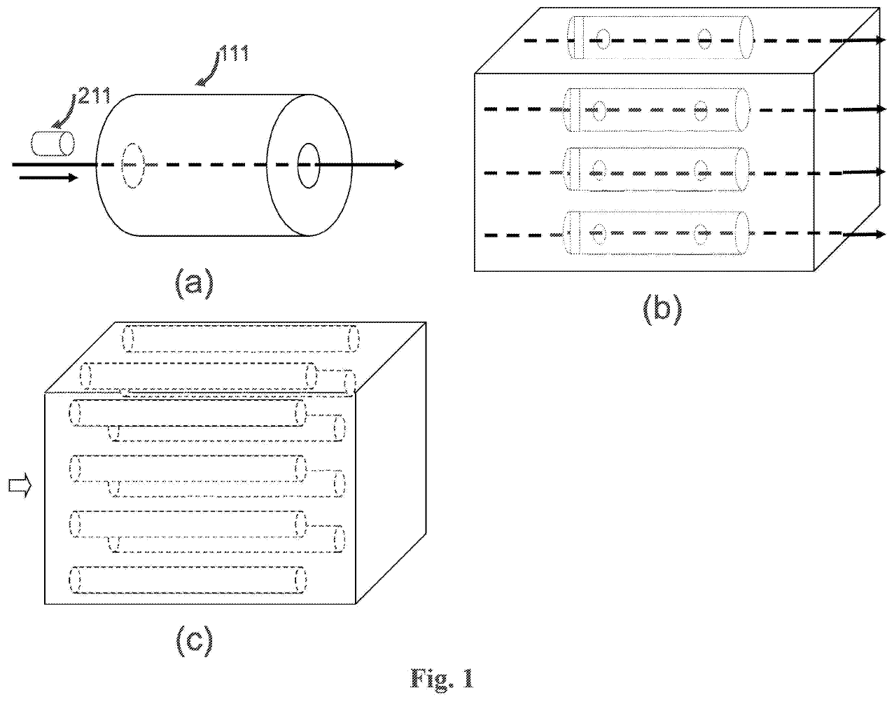

FIG. 1 (a) illustrates a set of traditional detection apparatus each of which relies on a single detection technology. As shown in FIG. 1 (a), current diagnosis devices detect a disease on a narrow focus and typically by one single technology (e.g., x-ray machine or NMR machine). FIG. 1 (b) and FIG. (c) are an illustration of a detection apparatus of this invention where multiple sub-equipment units are integrated into one piece of apparatus. As a result, the novel apparatus has a smaller size comparing to traditional devices.

FIG. 2 is a schematic illustration of a detection apparatus of this invention which comprises multiple sub-equipment units, a delivery system, and a central control system. The central control system comprises multiple processing units each of which can be a computer, data analysis unit, or display unit. The central control system is interacted with and used by multiple sub-equipment units. This resource sharing process can effectively reduce cost and size of the apparatus. The biological subject (e.g. a fluid sample) can flows to each sub-equipment units via the delivery system. The delivery system can also transport the biological subject to one or more desired sub-equipment for specific diagnosis purposes.

To enhance detection speed and sensitivity, a large number of micro-devices can be integrated into a single apparatus of this invention. Each micro-device can be an independent sub-equipment unit in the apparatus. To achieve the above requirements, the detection apparatus should be optimized with its surface area maximized to contact the biological sample and with large number of micro-devices integrated on the maximized surface.

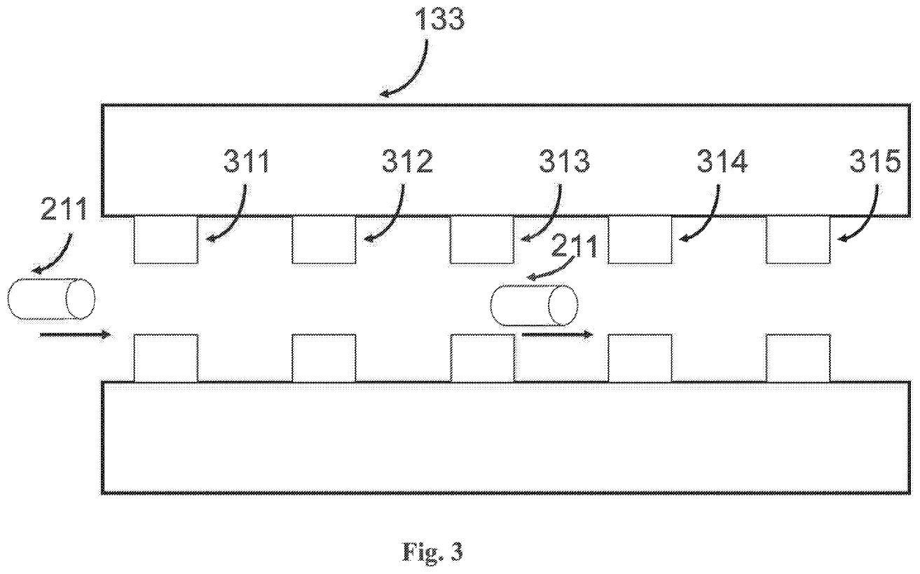

Instead of measuring a single property of a biological subject for disease diagnosis, various micro-devices can be integrated into a detection apparatus to detect multiple properties. Various micro-devices can constitute different sub-equipment units. FIG. 3 is a perspective, cross-sectional illustration of a disease detection apparatus of this invention 133 with multiple micro-devices 311, 312, 313, 314, and 315, of different detection probes in which a sample 211 such as a blood sample placed in it or moving through it can be tested for multiple properties including but not limited to mechanical properties (e.g., density, hardness and adhesion), thermal properties (e.g., temperature), biological properties, chemical properties (e.g., pH), physical properties, acoustical properties, electrical properties (e.g., surface charge, surface potential, and impedance), magnetic properties, electromagnetic properties, and optical properties.

As illustrated herein, it is desirable to optimize the detection apparatus design to maximize measurement surface area, since the greater the surface area, the greater number of micro-devices that can be placed on the detection apparatus to simultaneously measure the sample, thereby increasing detection speed and also minimizing the amount of sample needed for the test.

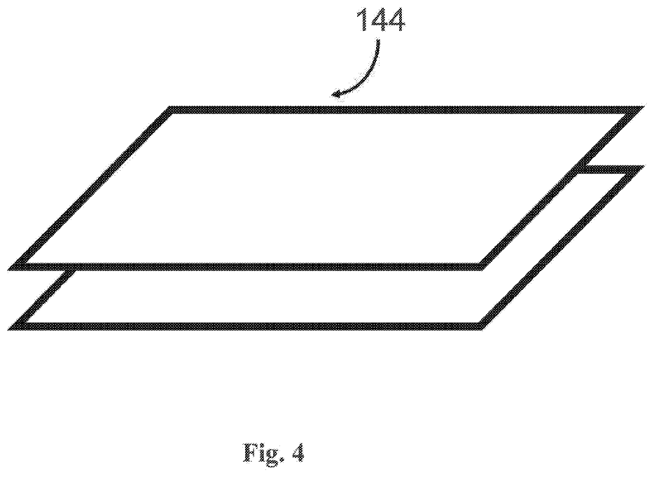

FIG. 4 is a perspective illustration of an apparatus or a sub-equipment unit of this invention. It includes two slabs separated by a narrow spacing with a sample such as a blood sample to be measured placed between the slabs, with multiple micro-devices placed at the inner surfaces of the slabs to measure one or more properties of the sample at microscopic levels.

Yet another aspect of this invention relates to a set of novel fabrication process flows for making micro-devices or sub-equipment units for disease detection purposes. Thus, a micro-device with two probes capable of measuring a range of properties (including mechanical and electrical properties) of biological samples is fabricated, using the above novel fabrication process flow.

Detection apparatus integrated with micro-devices disclosed in this application is fully capable of detecting pre-chosen properties on a single cell, a single DNA, a single RNA, or an individual, small sized biological matter level. In another further aspect, the invention provides the design, integration, and fabrication process flow of micro-devices capable of making highly sensitive and advanced measurements on very weak signals in biological systems for disease detection under complicated environment with very weak signal and relatively high noise background. Those novel capabilities using the class of micro-devices disclosed in this invention for disease detection include but not limited to making dynamic measurements, real time measurements (such as time of flight measurements, and combination of using probe signal and detecting response signal), phase lock-in technique to reduce background noise, and 4-point probe techniques to measure very weak signals, and unique and novel probes to measure various electronic, electromagnetic and magnetic properties of biological samples at the single cell (e.g., a telomere of DNA or chromosome), single molecule (e.g., DNA, RNA, or protein), single biological subject (e.g., virus) level.

For example, in a time of flight approach to obtain dynamic information on the biological sample (e.g., a cell, a substructure of a cell, a DNA, a RNA, or a virus), a first micro-device is first used to send a signal to perturb the biological subject to be diagnosed, and then a second micro-device is employed to accurately measure the response from the biological subject. In one embodiment, the first micro-device and the second micro-device are positioned with a desired or pre-determined distance L apart, with a biological subject to be measured flowing from the first micro-device towards the second micro-device. When the biological subject passes the first micro-device, the first micro-device sends a signal to the passing biological subject, and then the second micro-device detects the response or retention of the perturbation signal on the biological subject. From the distance between the two micro-devices, time interval, the nature of perturbation by the first micro-device, and measured changes on the biological subject during the time of flight, microscopic and dynamic properties of the biological subject can be obtained. In another embodiment, a first micro-device is used to probe the biological subject by applying a signal (e.g., an electronic charge) and the response from the biological subject is detected by a second micro-device as a function of time.

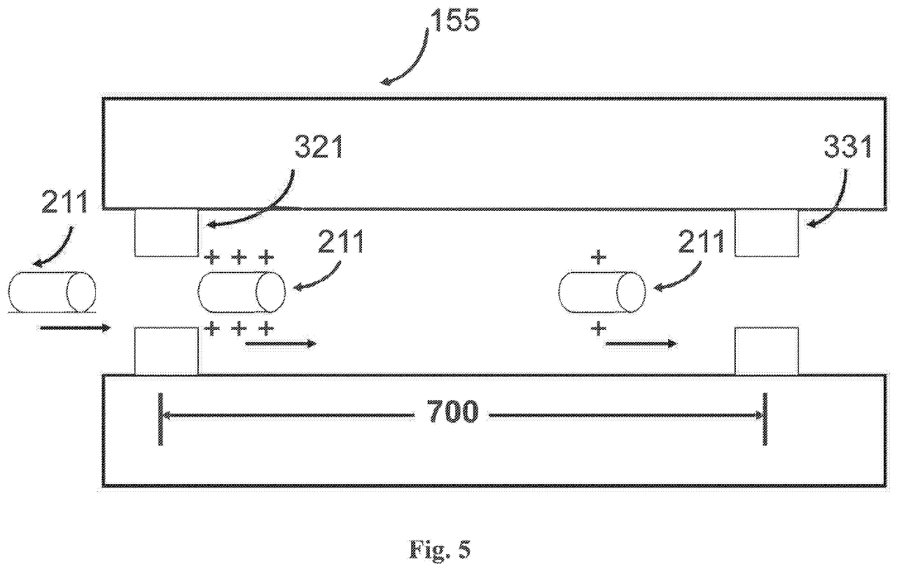

To further increase detection sensitivity, a novel detection process for disease detection is used, in which time of flight technique is employed. FIG. 5 is a perspective, cross-sectional illustration of detection apparatus 155 with multiple micro-devices 321 and 331 placed at a desired distance 700 for time of flight measurements to attain dynamic information on biological sample 211 (e.g., a cell) with enhanced measurement sensitivity, specificity, and speed. In this time of flight measurement, one or more properties of the biological sample 211 are first measured when the sample 211 passes the first micro-device 321. The same properties are then measured again when the sample 211 passes the second micro-device 331 after it has travelled the distance 700. The change in properties of sample 211 from at micro-device 321 to at micro-device 331 indicates how it reacts with its surrounding environment (e.g., a particular biological environment) during that period. It may also reveal information and provide insight on how its properties evolve with time. Alternatively, in the arrangement shown in FIG. 5, micro-device 321 could be used first as a probe to apply a probe signal (e.g., an electrical charge) to sample 211 as the sample passes the micro-device 321. Subsequently, the response of the sample to the probe signal can be detected by micro-device 331 as the sample passes it (e.g., change in the electrical charge on the sample during the flight). Measurements on biological sample 211 can be done via contact or non-contact measurements. In one embodiment, an array of micro-devices can be deployed at a desired spacing to measure properties of the biological subject over time.

The utilization of micro-devices (e.g., made by using the fabrication process flows of this invention) as discussed above and illustrated in FIG. 5 can be helpful for detecting a set of new, microscopic properties of a biological sample (e.g., a cell, a cell substructure, or a biological molecule such as DNA or RNA or protein) that have not been considered in existing detection technologies. Such microscopic properties can be thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, electrical, magnetic, electromagnetic, physical, or mechanical properties, or a combination thereof, of a biological sample that is a single biological subject (such as a cell, a cell substructure, a biological molecule—e.g., DNA, RNA, or protein—or a sample of a tissue or organ). It is known that biological matters include from basic bonding such as OH, CO, and CH bonding, to complex, three dimensional structures such as DNA and RNA. Some of them have a unique signature in terms of its electronic configuration. Some of them may have unique thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, electrical, magnetic, electromagnetic, physical, or mechanical properties and configurations, or a combination thereof. Normal biological subject and diseased biological subject may carry different signatures with respective to the above said properties. However, none of the above stated parameters or properties have been routinely used as a disease detection property. Using a disease detection apparatus including one or more apparatus of this invention, those properties can be detected, measured, and utilized as useful signals for disease detection, particularly for early stage detection of serious diseases such as cancer.

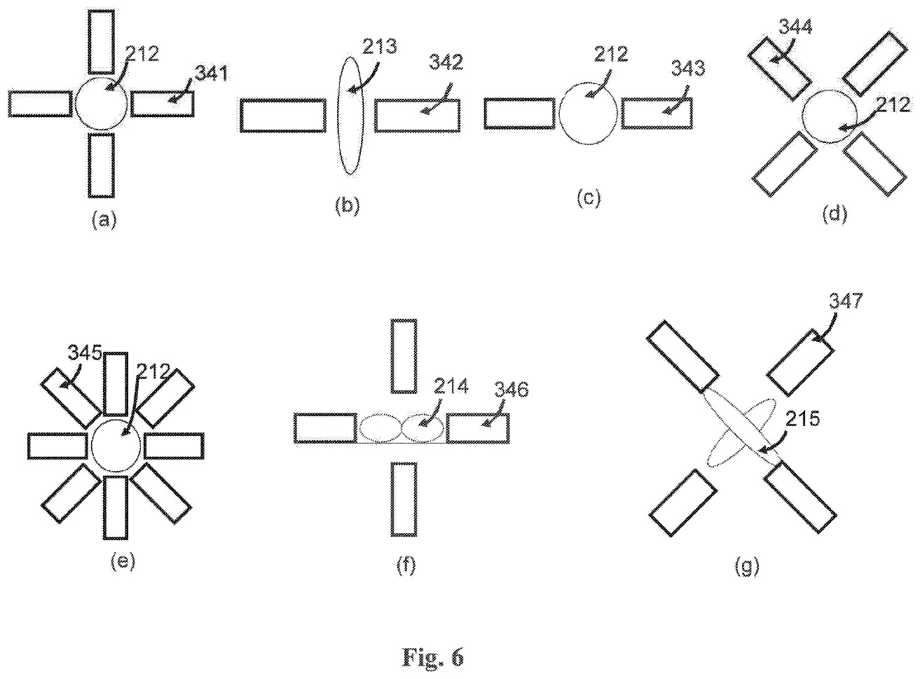

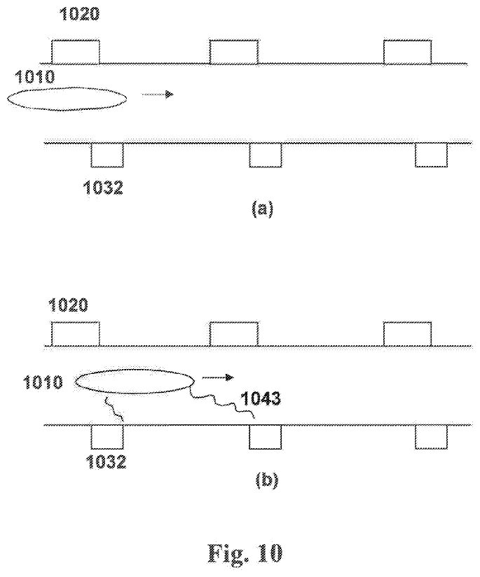

FIG. 6 is a perspective illustration of a novel set of microscopic probes 341, 342, 343, 344, 345, 346, and 347 designed and configured to detect various electronic, magnetic, or electromagnetic states, configurations, or other properties at microscopic level on biological samples 212, 213, 214, and 215, which can be a single cell, DNA, RNA, and tissue or sample. As an example, in terms of measuring electronic properties, the shapes of biological samples 212, 213, 214, and 215 in FIG. 10 may represent electronic monopole (sample 212), dipole (samples 213 and 214), and quadruple (sample 215). The micro-devices 341, 342, 343, 344, 345, 346, and 347 are optimized to maximize measurement sensitivity of those said parameters including but not limited to electronic states, electronic charge, electronic cloud distribution, electrical field, and magnetic and electromagnetic properties, and the micro-devices can be designed and arranged in three dimensional configurations. For some diseases such as cancer, it is likely that electronic states and corresponding electronic properties differ between normal and cancerous cells, DNA, RNA, and tissue. Therefore, by measuring electronic, magnetic and electromagnetic properties at microscopic levels including at cell, DNA, and RNA levels, disease detection sensitivity and specificity can be improved.

In addition to the above examples in measuring electrical properties (e.g., charge, electronic states, electronic charge, electronic cloud distribution, electrical field, current, and electrical potential, and impedance), mechanical properties (e.g., hardness, density, shear strength, and fracture strength) and chemical properties (e.g., pH) in a single cell, and in FIG. 6 for measuring electrical, magnetic or electromagnetic states or configurations of biological samples at cell and biological molecular (e.g., DNA, RNA, and protein) levels, other micro-devices are disclosed in this application for sensitive electrical measurements.

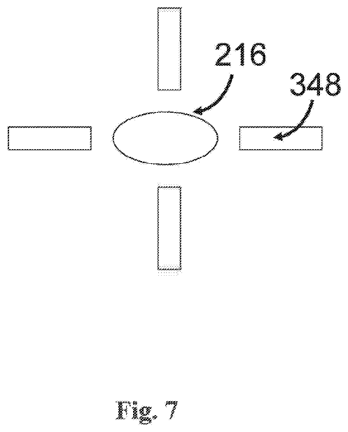

FIG. 7 is a perspective illustration of a four-point probe for detecting weak electronic signal in a biological sample such as a cell, where a four-point probe 348 is designed to measure electrical properties (impedance and weak electrical current) of a biological sample 216.

One of the key aspects of this invention is the design and fabrication process flows of micro-devices and methods of use the micro-devices for catching and/or measuring biological subjects (e.g., cells, cell substructures, DNA, and RNA) at microscopic levels and in three dimensional space, in which the micro-devices have micro-probes arranged in three dimensional manner with feature sizes as small as a cell, DNA, or RNA, and capable of trapping, sorting, probing, measuring, and modifying biological subjects. Such micro-devices can be fabricated using state-of-the-art microelectronics processing techniques such as those used in fabricating integrated circuits. Using thin film deposition technologies such as molecular epitaxial beam (MEB) and atomic layer deposition (ALD), film thickness as thin as a few monolayers can be achieved (e.g., 4 A to 10 A). Further, using electron beam or x-ray lithography, device feature size on the order of nanometers can be obtained, making micro-device capable of trapping, probing, measuring, and modifying a biological subject (e.g., a single cell, a single DNA or RNA molecule) possible.

Another aspect of this invention relates to micro-indentation probes and micro-probes for measuring a range of physical properties (such as mechanical properties) of biological subjects. Examples of the mechanical properties include hardness, shear strength, elongation strength, fracture stress, and other properties related to cell membrane which is believed to be a critical component in disease diagnosis.

Another novel approach provided by this invention is the use of phase lock-in measurement for disease detection, which reduces background noise and effectively enhances signal to noise ratio. Generally, in this measurement approach, a periodic signal is used to probe the biological sample and response coherent to the frequency of this periodic probe signal is detected and amplified, while other signals not coherent to the frequency of the probe signal is filtered out, which thereby effectively reduces background noise. In one of the embodiments in this invention, a probing micro-device can send a periodic probe signal (e.g., a pulsed laser team, a pulsed thermal wave, or an alternating electrical field) to a biological subject, response to the probe signal by the biological subject can be detected by a detecting micro-device. The phase lock-in technique can be used to filter out unwanted noise and enhance the response signal which is synchronized to the frequency of the probe signal. The following two examples illustrate the novel features of time of flight detection arrangement in combination with phase lock-in detection technique to enhance weak signal and therefore detection sensitivity in disease detection measurements.

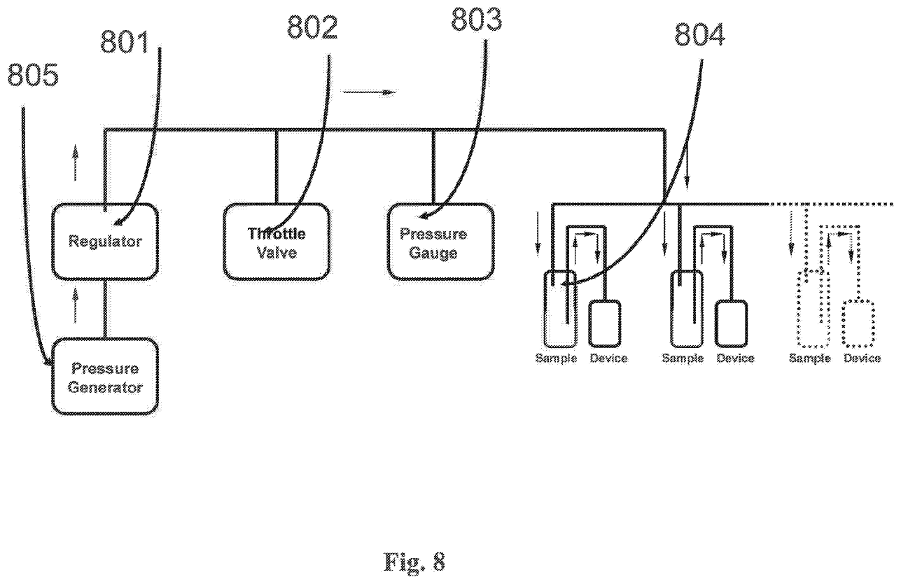

FIG. 8 illustrates a fluid delivery system that includes a pressure generator, a pressure regulator, a flow meter, a flow regulator, a throttle valve, a pressure gauge, and distributing kits. The pressure generator 805 sustains fluid with desired pressure, and the pressure is further regulated by the regulator 801 and then accurately manipulated by the throttle valve 802. Meanwhile, the pressure is monitored at real time and fed back to the throttle valve 802 by the pressure gauge 803. The regulated fluid is then in parallel conducted into the multiple devices where a constant pressure is needed to drive the fluid sample.

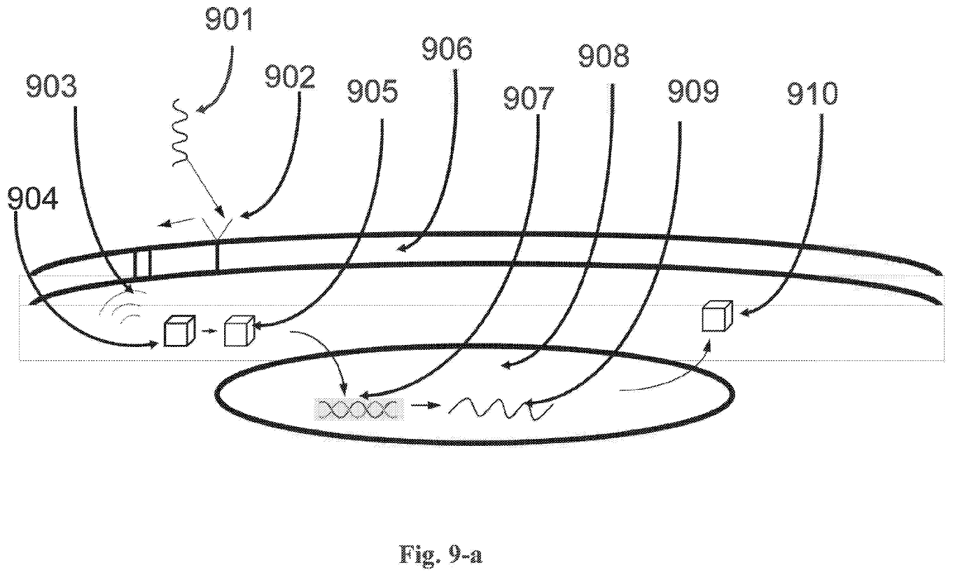

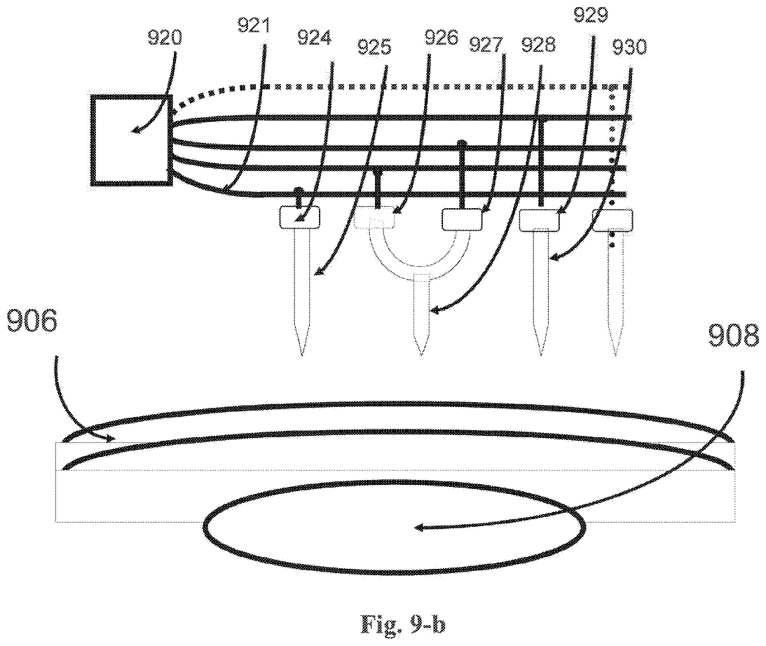

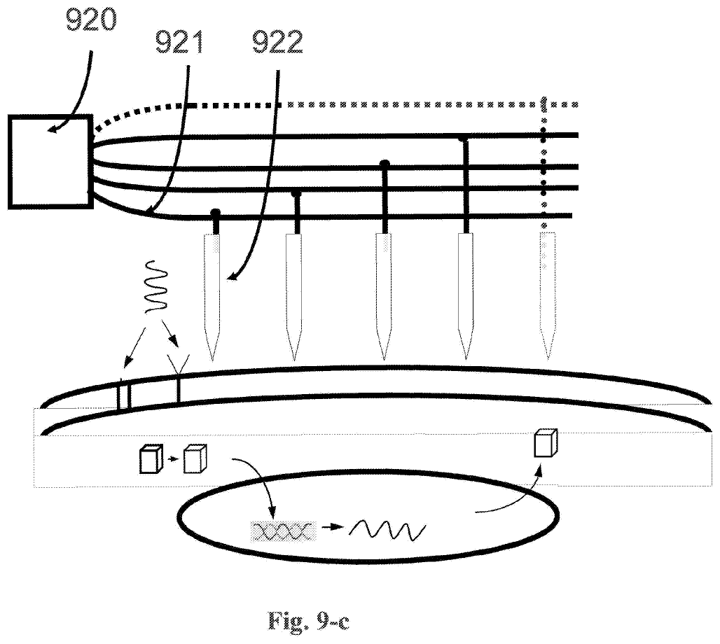

FIG. 9 illustrates how a micro-device in a disease detection apparatus of this invention can communicate, probe, detect, and optionally treat and modify biological subjects at a microscopic level. FIG. 9(a) illustrates the sequence of cellular events from signal recognition to cell fates determination. First, as the signals 901 are detected by receptors 902 on the cell surface, the cell will integrate and encode the signals into a biologically comprehensible message, such as calcium oscillation 903. Consequently, corresponding proteins 904 in the cell will interact with the message, then be modified and transform into ion-interacted proteins 905 accordingly. Through the translocation, these modified proteins 905 will pass the carried message to the nuclear proteins, and the controlled modification on nuclear proteins will modulate the expression of gene 907 which includes transcription, translation, epigenetic processes, and chromatin modifications. Through messenger RNA 909, the message is in turn passed to specific proteins 910, thereby changing their concentration—which then determines or regulates a cell's decision or activities, such as differentiation, division, or even death.

FIG. 9(b) illustrates a micro-device or sub-equipment of this invention which is capable of detecting, communicating with, treating, modifying, or probing a single cell, by a contact or non-contact means. The apparatus is equipped with micro-probes and micro-injectors which are addressed and modulated by the controlling circuitry 920. Each individual micro-injector is supplied with a separate micro-cartridge, which carries designed chemicals or compounds.

To illustrate how a micro-device can be used to simulate an intracellular signal, calcium oscillation is taken as an example mechanism. First, a Ca²⁺-release-activated channel (CRAC) has to be opened to its maximal extent, which could be achieved by various approaches. In an example of the applicable approaches, a biochemical material (e.g., thapsigargin) stored in the cartridge 924 is released by an injector 925 to the cell, and the CRAC will open at the stimulus of the biological subject. In another example of the applicable approaches, the injector 924 forces a specific voltage on cell membrane, which causes the CRAC to open as well.

The Ca²⁺ concentration of a solution in the injector 928 can be regulated as it is a desirable combination of a Ca²⁺-containing solution 926, and a Ca²⁺ free solution 927. While the injector 930 contains a Ca²⁺ free solution, then injectors 928 and 930 are alternately switched on and off at a desired frequency. As such, the Ca²⁺ oscillation is achieved and the content inside the cell membrane are then exposed to a Ca²⁺ oscillation. Consequently, the cell's activities or fate is being manipulated by the regulated signal generated by the apparatus.

Meanwhile, the cell's response (e.g., in the form of a thermal, optical, acoustical, mechanical, electrical, magnetic, electromagnetic property, or a combination thereof) can be monitored and recorded by the probes integrated in this apparatus.

FIG. 9(c) illustrates another design of a micro-device or sub-equipment which is able to setup communication with a single cell. The apparatus is equipped with micro-probes which are coated with biologically compatible compounds or elements, e.g., Ca, C, Cl, Co, Cu, H, I, Fe, Mg, Mn, N, O, P, F, K, Na, S, or Zn. These probes can generate oscillating chemical signals with such an element or compound to interact with the cell, and results into a response that affects the cell's activities or eventual fate as describe above. Likewise, this apparatus can probe and record the cell's response (e.g., in the form of an electrical, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical, mechanical property, or a combination thereof) as well.

As surface charge will affect the shape of a biological subject, by using novel and multiple plates, information on the shape and charge distribution of biological subjects can be obtained. The general principle and design of the micro-device can be extended to a broader scope, thereby making it possible to obtain other information on the biological subject via separation by applying other parameters such as ion gradient, thermal gradient, optical beam, or another form of energy.

FIG. 10 illustrates another micro-device or sub-equipment of this invention for detecting or measuring microscopic properties of a biological subject 1010 by utilizing a micro-device that includes a channel, a set of probes 1020, and a set of optical sensors 1032 (see, FIG. 10(a)). The detected signals by probes 1020 can be correlated to information including images collected by the optical sensors 1032 to enhance detection sensitivity and specificity. The optical sensors can be, e.g., a CCD camera, a florescence light detector, a CMOS imaging sensor, or any combination.

Alternatively, a probe 1020 can be designed to trigger optical emission such as florescence light emission 1043 in the targeted biological subject such as diseased cells, which can then be detected by an optical probe 1032 as illustrated in FIG. 10(c). Specifically, biological subjects can be first treated with a tag solution which can selectively react to diseased cells. Subsequently, upon reacting (contact or non-contact) with probe 1020, optical emissions from diseased cells occur and can be detected by optical sensors 1032. This novel process using the apparatus of this invention is more sensitive than such conventional methods as traditional florescence spectroscopy as the emission trigger point is directly next to the optical probe and the triggered signal 1043 can be recorded in real time and on-site, with minimum loss of signal.

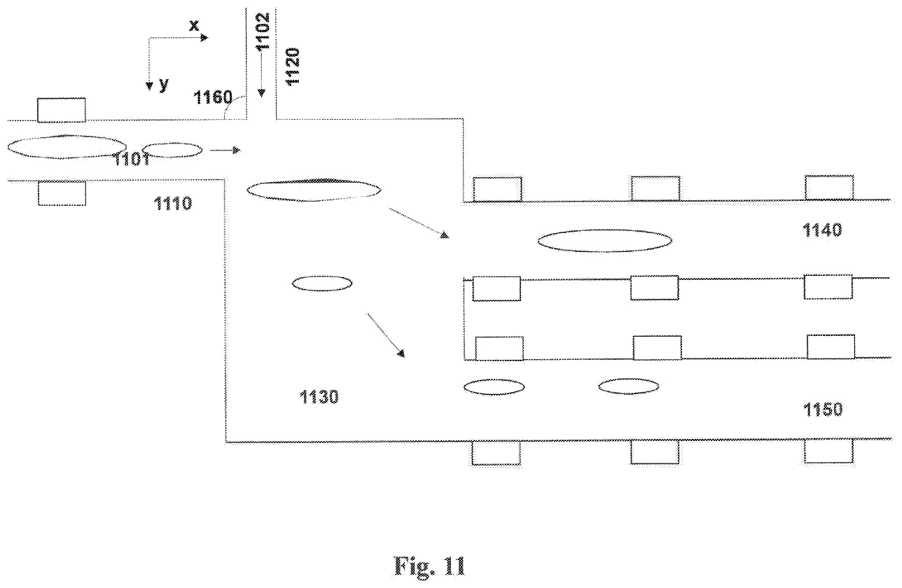

FIG. 11 illustrates another embodiment of the apparatus of this invention, which can be used to separate biological subjects of different geometric size and detect their properties respectively. It includes at least an entrance channel 1110, a disturbing fluid channel 1120, an accelerating chamber 1130, and two selecting channels 1140 and 1150. The angle between 1120 and 1110 is between 0° and 180°. The biological subject 1101 flows in the x-direction from 1110 to 1130. The biocompatible distribution fluid 1102 flows from 1120 to 1130. Then the fluid 1102 will accelerate 1101 in y-direction. However, the acceleration correlates with the radius of the biological subjects and the larger ones are less accelerated than the small ones. Thus, the larger and smaller subjects are separated into different channels. Meanwhile, probes can be optionally assembled aside the sidewall of 1110, 1120, 1130, 1140, and 1150. They could detect electrical, magnetic, electromagnetic, thermal, optical, acoustical, biological, chemical, physical, mechanical properties, or combinations thereof at the microscopic level. In the meantime, if desired, a cleaning fluid can also be injected into the system for dissolving and/or cleaning biological residues and deposits (e.g., dried blood and protein) in the narrow and small spaces in the apparatus, and ensuring smooth passage of a biological subject to be tested through the apparatus.

The channel included in the apparatus of this invention can have a width of, e.g., from 1 nm to 1 mm. The apparatus should have at least one inlet channel and at least two outlet channels.

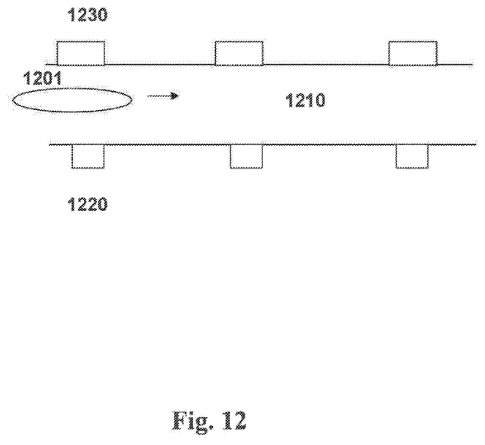

FIG. 12 shows another micro-device or sub-equipment of this invention with an acoustic detector 1220 for measuring the acoustic property of a biological subject 1201. This device includes a channel 1210, and at least an ultrasonic emitter and an ultrasonic receiver installed along the sidewall of the channel. When the biological subject 1201 passes through the channel 1210, the ultrasonic signal emitted from 1220 will be received after carrying information on 1201 by the receiver 1230. The frequency of the ultrasonic signal can be, e.g., from 2 MHz to 10 GHz, and the trench width of the channel can be, e.g., from 1 nm to 1 mm. The acoustic transducer (i.e., the ultrasonic emitter) can be fabricated using a piezo-electrical material (e.g., quartz, berlinite, gallium, orthophosphate, GaPO₄, tourmalines, ceramics, barium, titanate, BatiO₃, lead zirconate, titanate PZT, zinc oxide, aluminum nitride, and polyvinylidene fluorides).

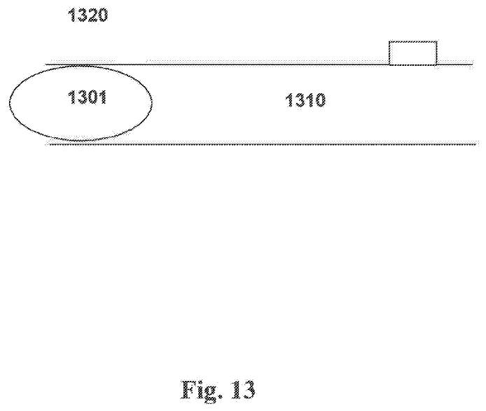

FIG. 13 shows another apparatus of this invention that includes a pressure detector for biological subject 1301. It includes at least one channel 1310 and whereon at least one piezo-electrical detector 1320. When the biologic subject 1301 passes through the channel, the piezo-electrical detector 1320 will detect the pressure of 1301, transform the information into an electrical signal, and send it out to a signal reader. Likewise, the trench width in the apparatus can be, e.g., from 1 nm to 1 mm, and the piezo-electrical material can be, e.g., quartz, berlinite, gallium, orthophosphate, GaPO₄, tourmalines, ceramics, barium, titanate, BatiO₃, lead zirconate, titanate PZT, zinc oxide, aluminum nitride, or polyvinylidene fluorides.

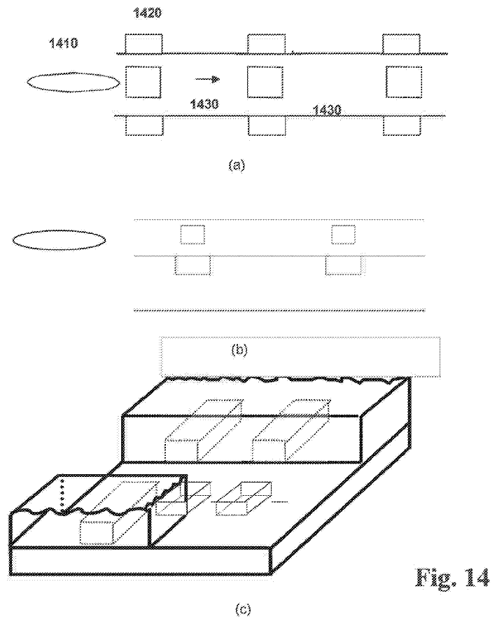

FIG. 14 shows another apparatus of this invention that include a concave groove 1430 between a probe couple, in the bottom or ceiling of the channel. When a biological subject 1410 passes through, the concave 1430 can selectively trap the biological subject with particular geometric characteristics and makes the probing more efficiently. The shape of concave's projection can be rectangle, polygon, ellipse, or circle. The probe could detect electrical, magnetic, electromagnetic, thermal, optical, acoustical, biological, chemical, physical, mechanical properties, or combinations thereof. Similarly, the trench width can be, e.g., from 1 nm to 1 mm. FIG. 14(a) is an up-down view of this apparatus, FIG. 14(b) is a side view, whereas FIG. 14(c) is a perspective view.

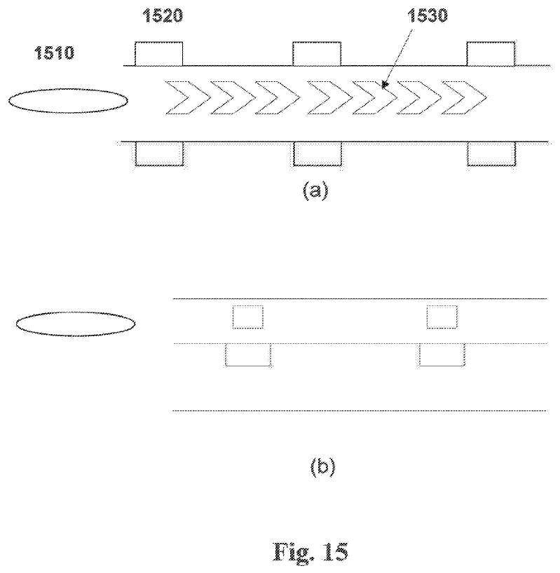

FIG. 15 is another apparatus of this invention that also includes concave grooves 1530 (of a different shape from those shown in FIG. 14) on the bottom or ceiling of the channel. When a biological subject 1510 passes through, the concave grooves 1530 will generate a turbulent fluidic flow, which can selectively trap the micro-biological subjects with particular geometric characteristics. The probe could detect, e.g., electrical, magnetic, electromagnetic, thermal, optical, acoustical, biological, chemical, physical, mechanical properties, or a combination thereof. The depth of the concave groove can be, e.g., from 10 nm to 1 mm, and the channel width can be, e.g., from 1 nm to 1 mm.

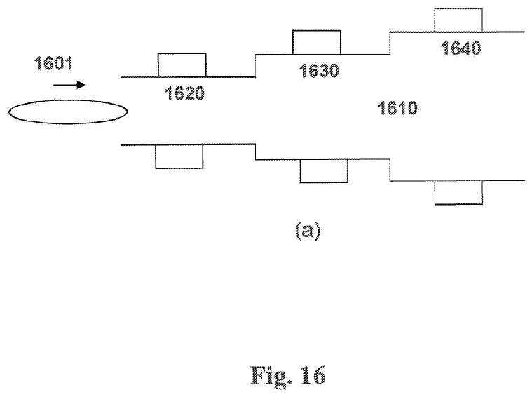

FIG. 16 illustrated a micro-device with a stepped channel 1610. When a biological subject 1601 passes through the channel 1610, probe couples of different distances can be used to measure different microscopic properties, or even the same microscopic at different sensitivity at various steps (1620, 1630, 1640) with probe aside each step. This mechanism can be used in the phase lock-in application so that signal for the same microscopic property can be accumulated. The probes can detect or measure microscopic electrical, magnetic, electromagnetic, thermal, optical, acoustical, biological, chemical, physical, mechanical properties, or combinations thereof.

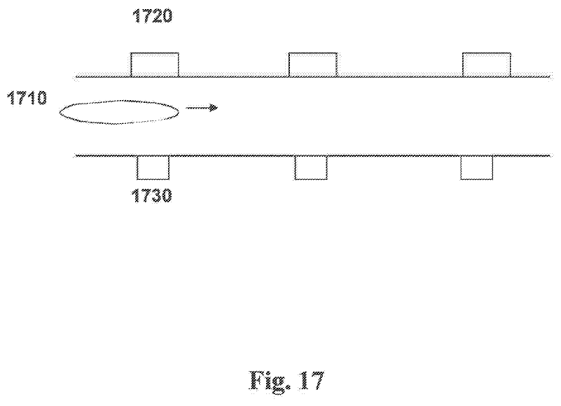

FIG. 17 illustrates another apparatus of this invention with thermal meters 1730. It includes a channel, a set of probes 1720, and a set of thermal meters 1730. The thermal meters 1730 can be an infrared sensor, a transistor sub-threshold leakage current tester, or thermistor.

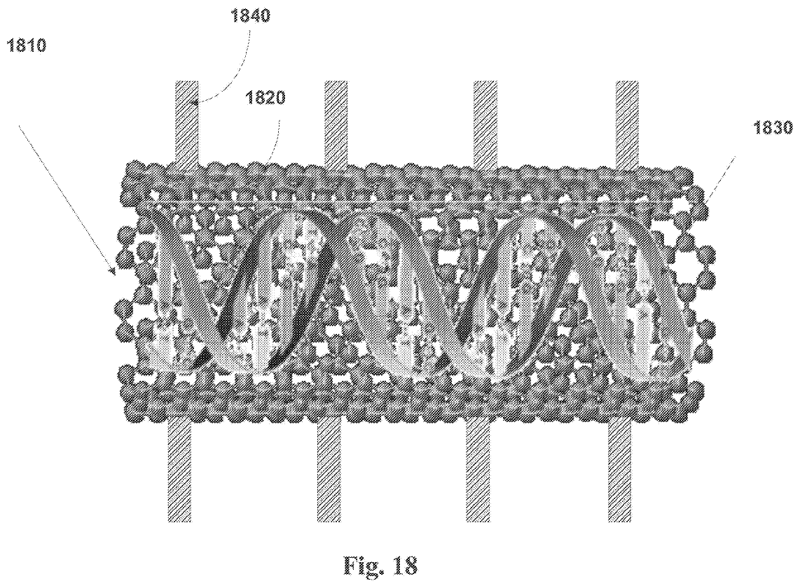

FIG. 18 illustrates a specific apparatus of this invention which includes carbon a nano-tube 1820 with a channel 1810 inside, probes 1840 which can detect at the microscopic level an electrical, magnetic, electromagnetic, thermal, optical, acoustical, biological, chemical, physical, or mechanical property, or a combination thereof. The carbon nano-tube 1820 as shown contains a double-helix DNA molecule 1830. The carbon nano-tube can force and sense electrical signals by the probes 1840 aside. The diameter of the carbon nano tube diameter can be, e.g., from 0.5 nm to 50 nm, and its length can range from, e.g., 5 nm to 10 mm.

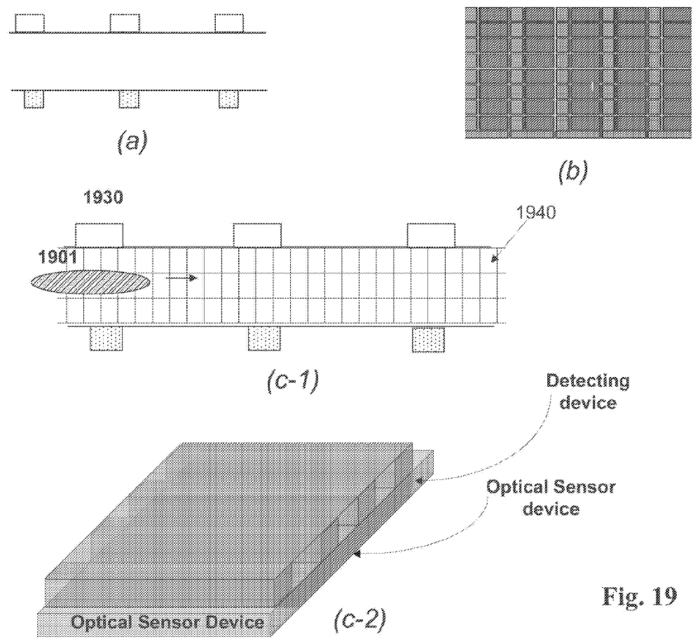

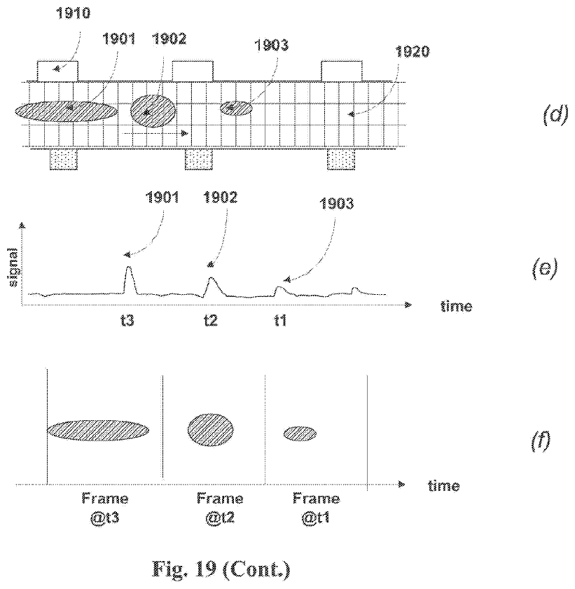

FIG. 19 shows an integrated apparatus of this invention that includes a detecting device (shown in FIG. 19(a)) and an optical sensor (shown in FIG. 19(b)) which can be, e.g., a CMOS image sensor (CIS), a Charge-Coupled Device (CCD), a florescence light detector, or another image sensor. The detecting device comprises at least a probe and a channel, and the image device comprises at least 1 pixel. FIG. 19(c-1) and FIG. 19(c-2) illustrate the device with the detecting device and optical sensor integrated. As illustrated in FIG. 19(d), when biological subjects 1901, 1902, 1903 pass through, the probe 1910 in the channel 1920, its electrical, magnetic, electromagnetic, thermal, optical, acoustical, biological, chemical, physical, mechanical property or a combination thereof could be detected by the probe 1910 (see FIG. 19(e)), meanwhile its image could be synchronously recorded by the optical sensor (FIG. 19(f)). Both the probed signal and image are combined together to provide a diagnosis and enhanced detection sensitivity and specificity. Such a detecting device and an optical sensing device can be designed in a system-on-chip or be packaged into one chip.

FIG. 20 shows a micro-device or sub-equipment with a detecting micro-device (FIG. 20(a)) and a logic circuitry (FIG. 20(b)). The detecting device comprises at least a probe and a channel, and the logic circuitry comprises an addressor, an amplifier, and a RAM. When a biological subject 2001 passes through the channel, its property could be detected by the probe 2030, and the signal can be addressed, analyzed, stored, processed, and plotted in real time. FIG. 20(c-1) and FIG. 20(c-2) illustrate the device with detecting device and Circuitry integrated. Similarly, the detecting device and the integrated circuit can be designed in a System-on-Chip or be packaged into one chip.

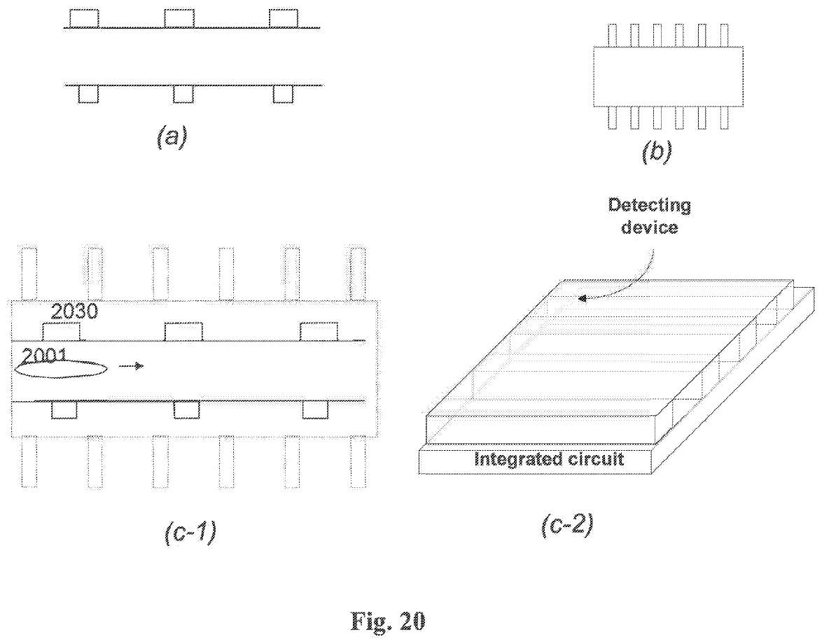

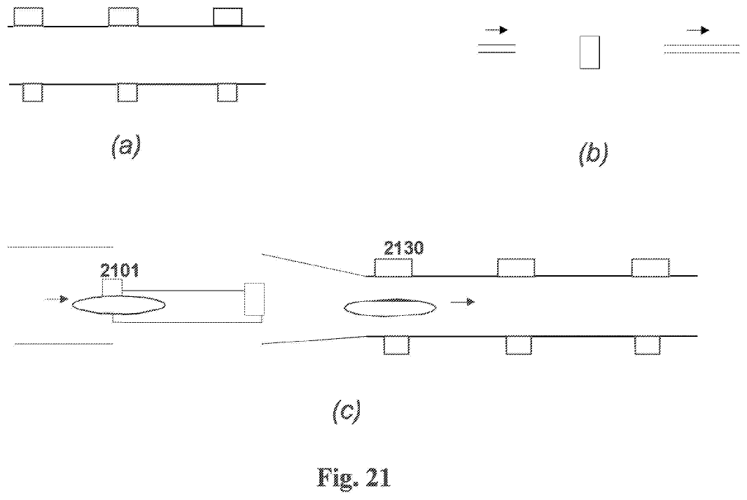

FIG. 21 shows a micro-device or sub-equipment of this invention that comprises a detecting device (FIG. 21(a)) and a filter (FIG. 21(b)). When a biological subject 2101 passes through the device, a filtration is performed in the filter, and irrelevant objects can be removed. The remaining subjects' property can then be detected by the probe device (FIG. 20(a)). The filtration before probing will enhance the precision of the device. The width of the channel can also range, e.g., from 1 nm to 1 mm.

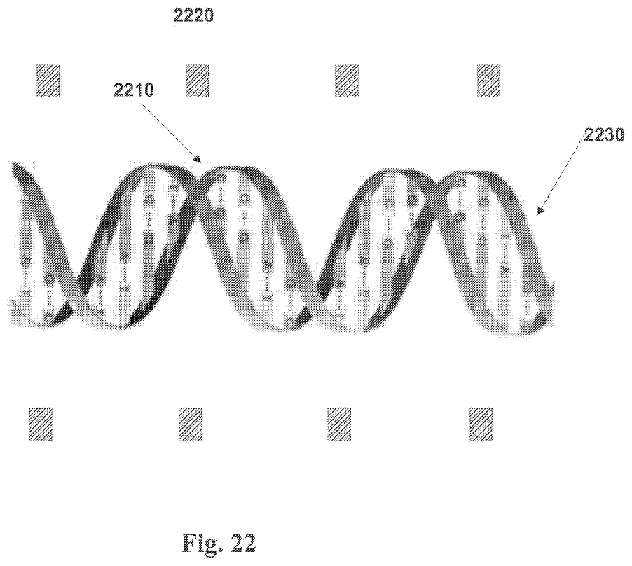

FIG. 22 shows the geometric factors of DNA 2230 such as spacing in DNA's minor groove (2210) have an impact on spatial distribution of electrostatic properties in the region, which in turn may impact local biochemical or chemical reactions in the segment of this DNA. By probing, measuring, and modifying spatial properties of DNA (such as the spacing of minor groove) using the disclosed detector and probe 2220, one may detect properties such as defect of DNA, predict reaction/process at the segment of the DNA, and repair or manipulate geometric properties and therefore spatial distribution of electrostatic field/charge, impacting biochemical or chemical reaction at the segment of the DNA. For example, tip 2220 can be used to physically increase spacing of minor groove 2210.

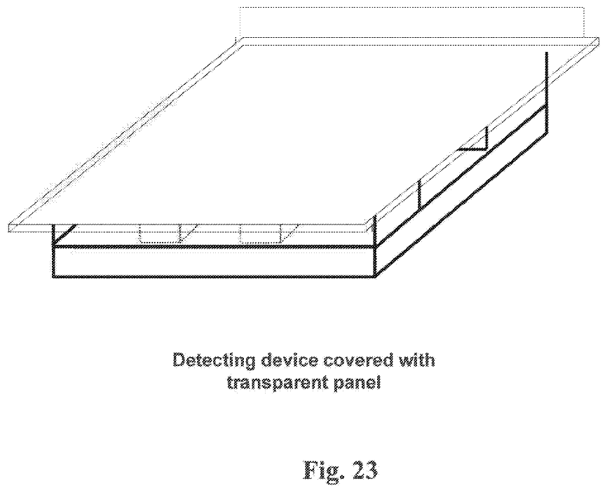

FIG. 23 shows the fabrication process for an apparatus of this invention that has a flat cover atop of trench to form a channel. This will eliminate the need for coupling two trenches to form a channel, which can be tedious for requiring perfect alignment. The cover can be transparent and allow observation with a microscope. It can comprise or be made of silicon, SiGe, SiO₂, various types of glass, or Al₂O₃.

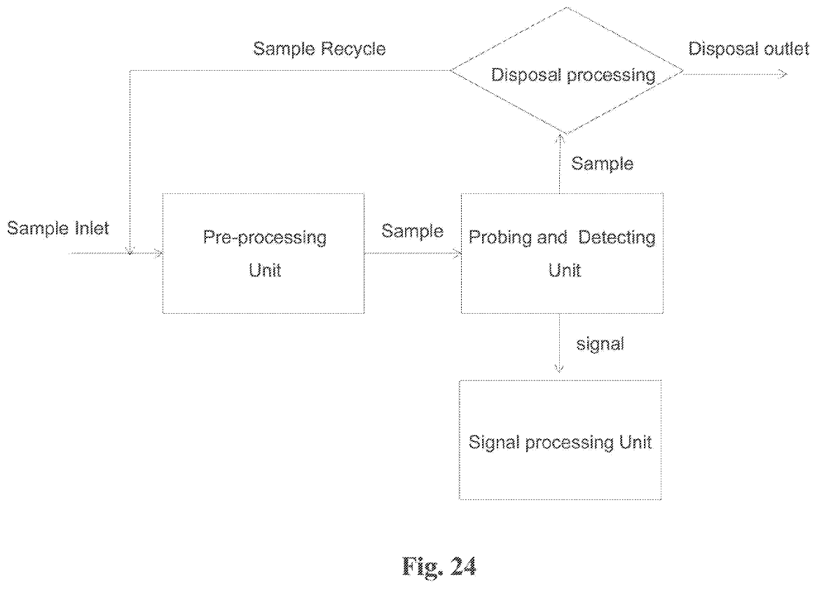

FIG. 24 is a diagram of an apparatus of this invention for detecting a disease in a biological subject. This apparatus includes a pre-processing unit, a probing and detecting unit, a signal processing, and a disposal processing unit.

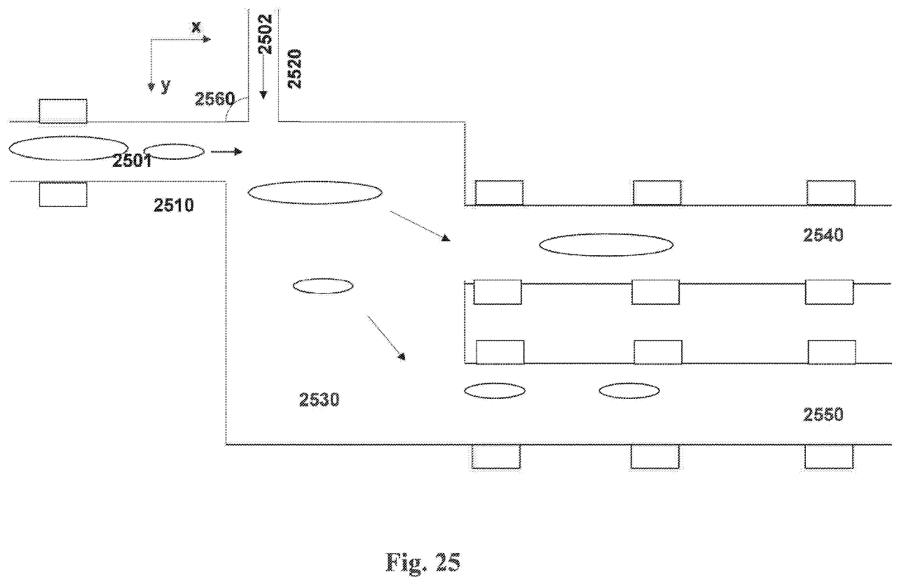

FIG. 25 shows an example of a sample filtration sub-unit in the pre-processing unit, which can separate the cells with different dimensions or sizes. This device comprises at least one entrance channel 2510, one disturbing fluid channel 2520, one accelerating chamber 2530, and two selecting channels (2540 and 2550). The angle 2560 between 2520 and 2510 ranges from 0° to 180°.

The biological subject 2501 flows in the x direction from the entrance channel 2510 to the accelerating chamber 2530. A bio-compatible fluid 2502 flows from disturbing fluid channel 2520 to the accelerating chamber 2530, it then accelerates the biological subject 2501 in the y-direction. The acceleration correlates with the radius of the biological subject and the larger ones are less accelerated than the smaller ones. Then, the larger and smaller subjects are separated into different selecting channels. Meanwhile, probes can be optionally assembled on the sidewalls of the channels 2510, 2520, 2530, 2540, and 2550. The probes could detect, at the microscopic level, electrical, magnetic, electromagnetic, thermal, optical, acoustical, biological, chemical, biochemical, electro-mechanical, electro-chemical, electro-chemical-mechanical, physical, mechanical properties, or combinations thereof.

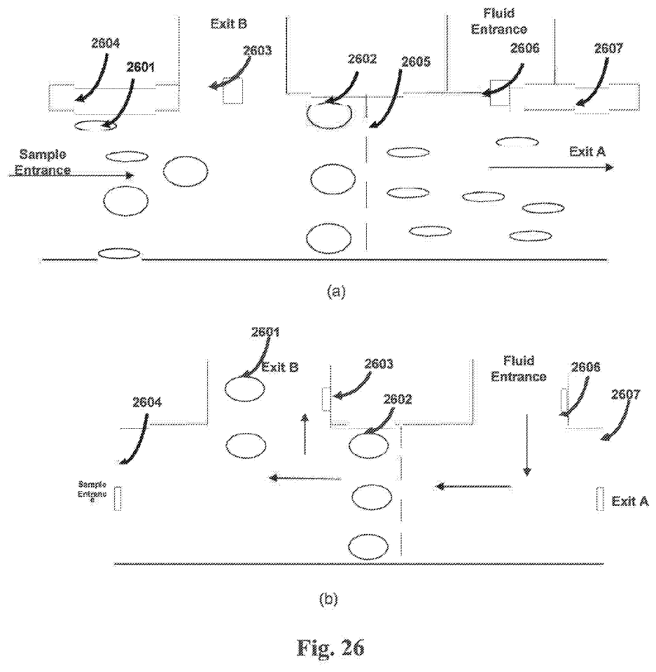

FIG. 26 is a diagram of another example of a sample filtration unit in the apparatus of this invention. 2601 represents small cells, while 2602 represents large cells. When a valve 2604 is open and another valve 2603 is closed, biological subjects (2601 and 2602) flow towards exit A. Large cells that have larger size than the filtration hole are blocked against exit A, while small cells are flushed out through exit A. The entrance valve 2604 and exit A valve 2607 are then closed, and a bio-compatible fluid is injected through the fluid entrance valve 2606. The fluid carries big cells are flushed out from exit B. The larger cells are then analyzed and detected in the detection part of the invention.

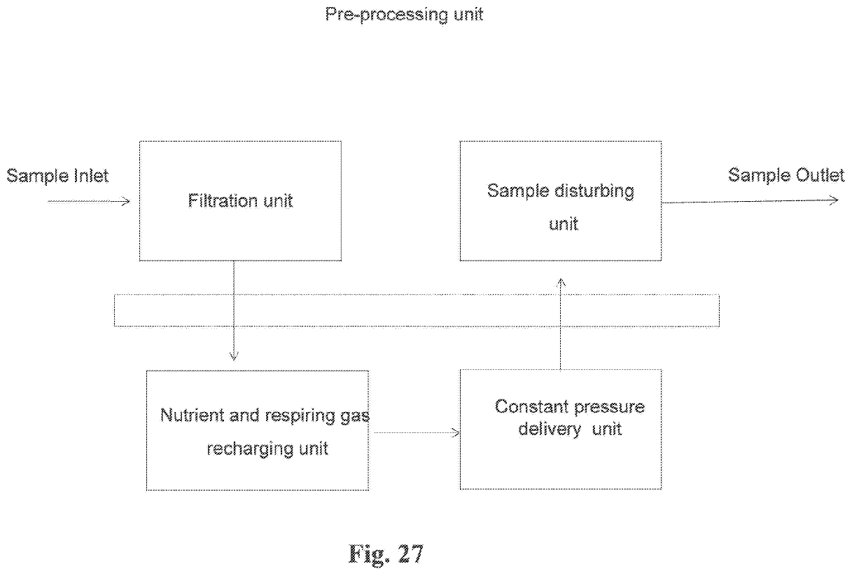

FIG. 27 is a diagram of a pre-processing unit of an apparatus of this invention. This unit includes a sample filtration unit, a recharging unit or system for recharging nutrient or gas into the biological subject, a constant pressure delivery unit, and a sample pre-probing disturbing unit.

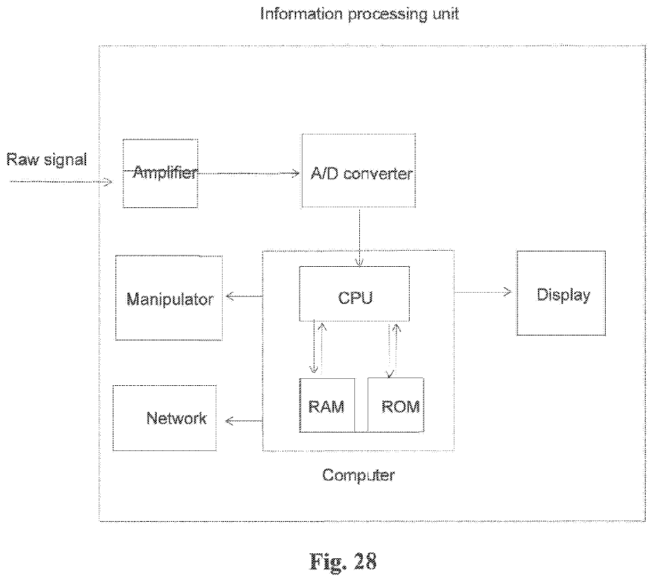

FIG. 28 is a diagram of an information or signal processing unit of an apparatus of this invention. This unit includes an amplifier (such as a lock-in amplifier) for amplifying the signal, an A/D converter, and a micro-computer (e.g., a device containing a computer chip or information processing sub-device), a manipulator, a display, and network connections.

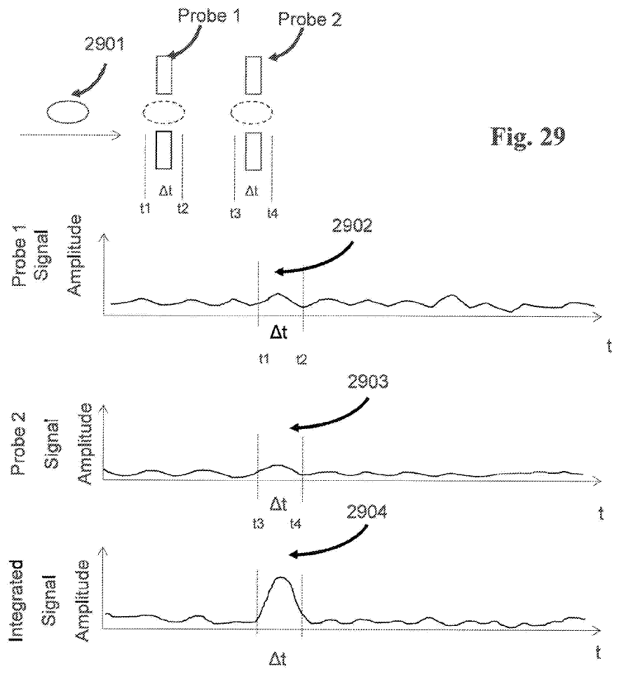

FIG. 29 shows the integration of multiple signals which results in cancellation of noise and enhancement of signal/noise ratio. In this Figure, a biological 2901 is tested by Probe 1 during Δt between t1 and t2, and by Probe 2 during Δt between t3 and t4. 2902 is 2901's tested signal from Probe 1, and 2903 is from Probe 2. Signal 2904 is the integration result from signal 2902 and 2903. The noise cancels out each other in certain extent and results in an improved signal strength or signal/noise ratio. The same principle can be applied to data collected from more than more than 2 micro-devices or probing units.

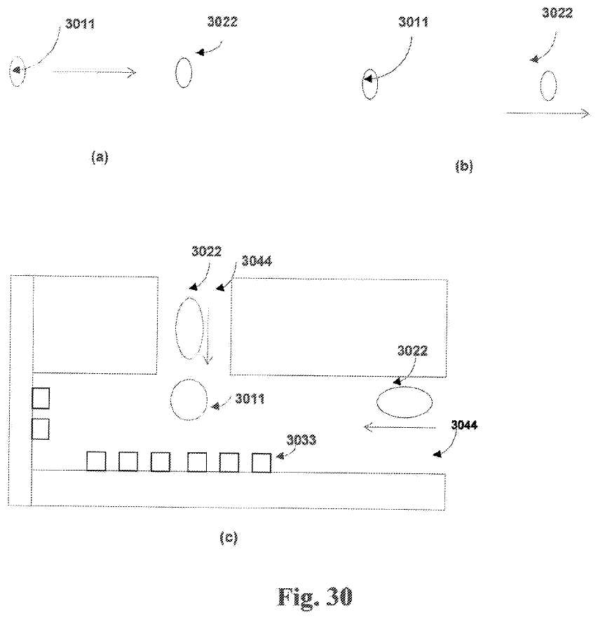

FIG. 30 shows a novel disease detection method of this invention in which at least one probe object is launched at a desired speed and direction toward a biological subject, resulting in a collision. The response(s) by the biological subject during and/or after the collision is detected and recorded, which can provide detailed and microscopic information on the biological subject such as weight, density, elasticity, rigidity, structure, bonding (between different components in the biological subject), electrical properties such as electrical charge, magnetic properties, structural information, and surface properties. For example, for a same type of cell, it is expected that a cancerous cell will experience a smaller traveling distance after the collision than that of a normal cell due to its denser, greater weight, and possibly larger volume. As shown in FIG. 30(a), a probe object 3011 is launched towards a biological subject 3022. After the collision with the probe object 3011, the biological subject 3022 may be pushed (scattered) out a distance depending on its properties as shown FIG. 30(b).

FIG. 30(c) shows a schematic of a novel disease detection device with a probe object launch chamber 3044, an array of detectors 3033, a probe object 3022 and a biological subject to be tested 3011. In general, a test object can be an inorganic particle, an organic particle, a composite particle, or a biological subject itself. The launch chamber comprises a piston to launch the object, a control system interfaced to an electronic circuit or a computer for instructions, and a channel to direct the object.

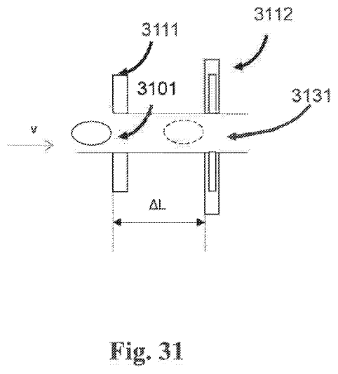

FIG. 31 illustrates a method for detecting a disease in a biological subject. A biological subject 3101 passes through the channel 3131 at a speed v, and probe 3111 is a probe which can grossly detect the properties of the biological subject at high speed.

Probe 3112 is a fine probing device which is coated by a piezo-electrical material. There is a distance ΔL between probe 3111 and probe 3112.

When the biological subjects are tested when getting through 3111, if the entity is identified to be a suspected abnormal one, the system would trigger the piezo-electrical probe 3112 to stretch into the channel and probe particular properties after a time delay of Δt. And probe 3112 retracts after the suspected entity passed through.

The probing device is capable of measuring at the microscopic level an electrical, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical property, or a combination thereof, of the biological subject.

The width of the micro-channel can range from about 1 nm to about 1 mm.

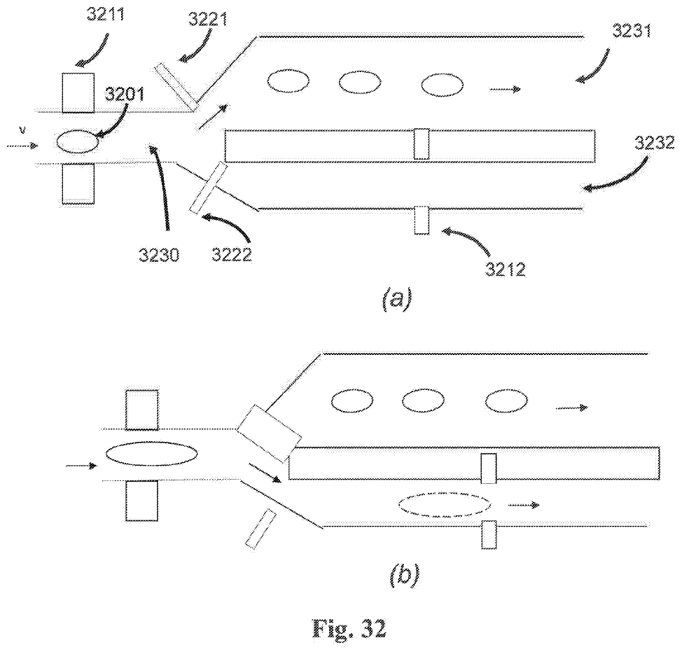

FIG. 32 shows a process of detecting a disease in a biological subject. A biological subject 3201 passes through the channel 3231 at a speed v. Probe 3211 is a probe which can grossly detect the properties of the biological subject at high speed. 3221 and 3222 are piezo-electrical valves to control the micro-channel 3231 and 3232. 3212 is a fine probing device which can probe biological properties more particularly. 3231 is flush channel to rush out normal biological subjects. 3232 is detection channel where the suspected entities are fine detected in this channel.

When a biological subject is tested while getting through 3211, if it is normal, the valve 3221 of the flush channel is open, while the detection channel valve 3222 is closed, the biological subject is flushed out without a time-consuming fine detection.

When the biological subject is tested while getting through 3211, if it is suspected to be abnormal or diseased, the valve 3221 of the flush channel is closed, while the detection channel valve 3222 is open, the biological subject is conducted to the detection channel for a more particular probing.

The width of the micro-channel can range from about 1 nm to about 1 mm.

The probing device is capable of measuring at the microscopic level an electrical, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical property, or a combination thereof, of the biological subject.

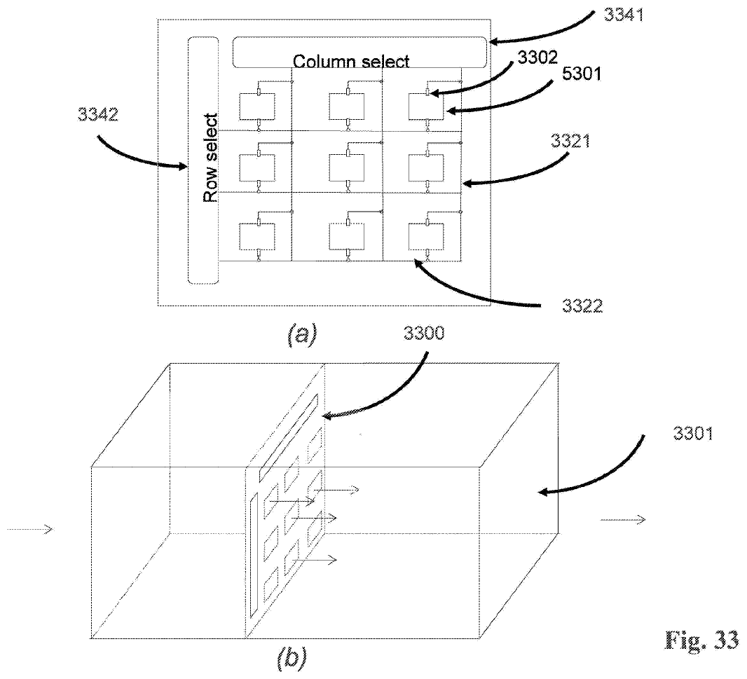

FIG. 33 illustrates an arrayed biological detecting device. As shown in FIG. 33(a), 3301 are arrayed micro-channels which can get through the fluidics and biological subjects. 3302 are probing devices embedded aside the channels. The sensors are wired by bit-lines 3321 and word-lines 3322. The signals are applied and collected by the decoder R\row-select 3342 and decoder column select 3341. As illustrated in FIG. 33(b), the micro-channel arrayed biological detecting device 3300 can be embedded in a macro-channel 3301. The micro-channel's dimension ranges from about 1 um to about 1 mm. The shape of the micro-channel can be rectangle, ellipse, circle, or polygon.

The probing device is capable of measuring at the microscopic level an electrical, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical property, or a combination thereof, of the biological subject.

FIG. 34 illustrates a device of the current invention for disease detection. 3401 is inlet of the detecting device, and 3402 is the outlet of the device. 3420 is the channel where the biological subjects pass through. 3411 is the optical component of the detecting device.

As illustrated in FIG. 34(b), the optical component 3411 consists of an optical emitter 3412 and an optical receiver 3413. The optical emitter emits an optical pulse (e.g. laser beam pulse), when the biological subject 3401 passing through the optical component, and the optical sensor detects the diffraction of the optical pulse, then identify the morphology of the entity.

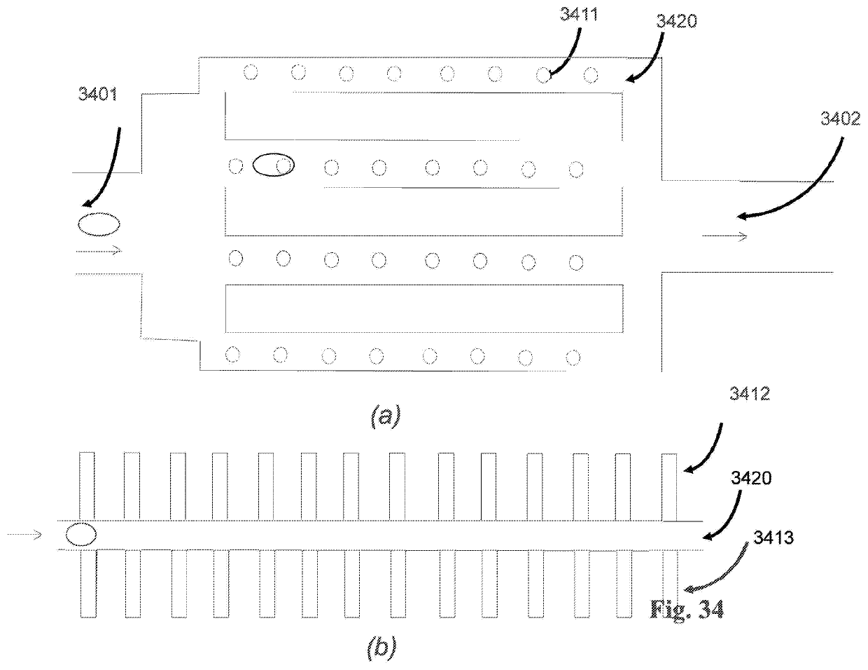

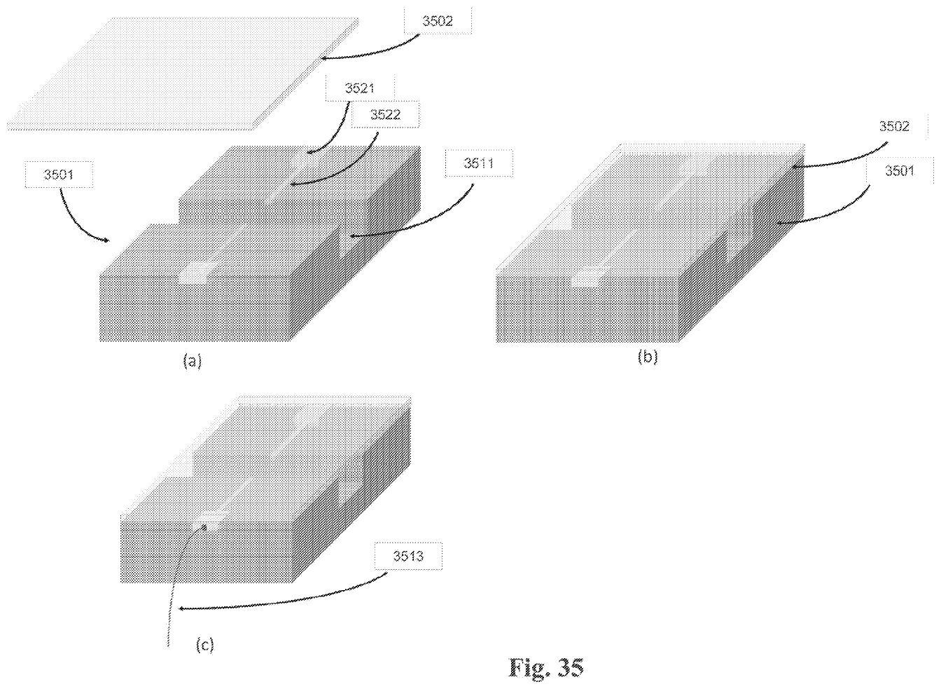

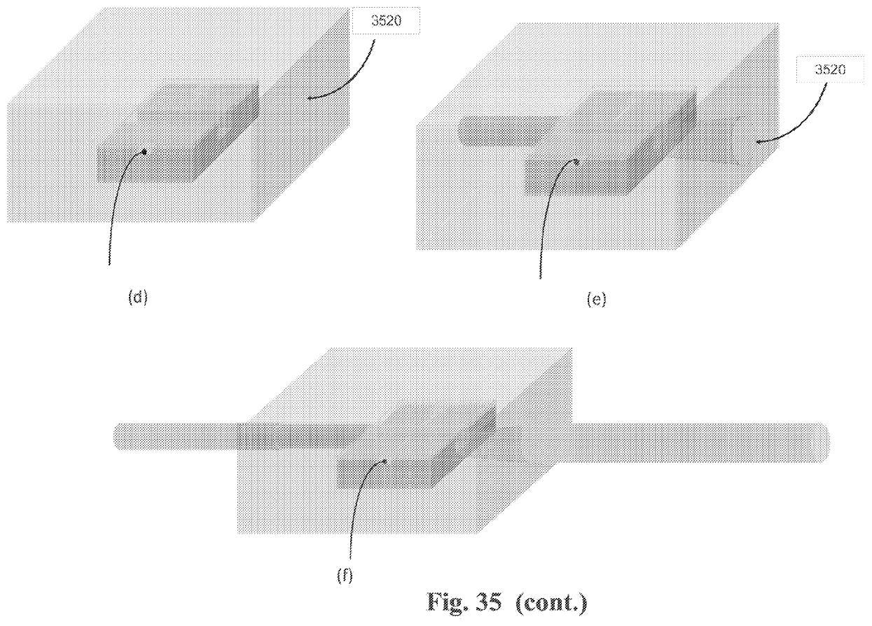

FIG. 35 shows an example of the apparatus of this invention packaged and ready for integration with a sample delivery system and data recording device. As illustrated in FIG. 35(a), the device 3501 is fabricated by micro-electronics processes described herein and has at least a micro-trench 3511, a probe 3522, and a bonding pad 3521. The surface of the device's top layer can include SixOyNz, Si, SixOy, SixNy, or a compound containing the elements of Si, O, and N. Component 3502 is a flat glass panel. In FIG. 35(b), the flat panel 3502 is shown to be bonded with micro-device 3501 on the side of micro-trench. The bonding can be achieved by a chemical, thermal, physical, optical, acoustical, or electrical means, or any combination thereof. FIG. 35(c) shows a conductive wire being bonded with the bonding pad from the side of the pads. As illustrated in FIG. 35(d), the device 3501 is then packaged in a plastic cube with only conducting wires exposed. In FIG. 35(e), a conical channel 3520 is carved through packaging material and connecting the internal channel of the device. As illustrated in FIG. 35(f), the larger opening mouth of the conical channel makes it operational and convenient to mount a sample delivery injector with the device, thereby better enabling the delivery of sample from an injector with relatively large size of injector needle into device with relatively small channels.

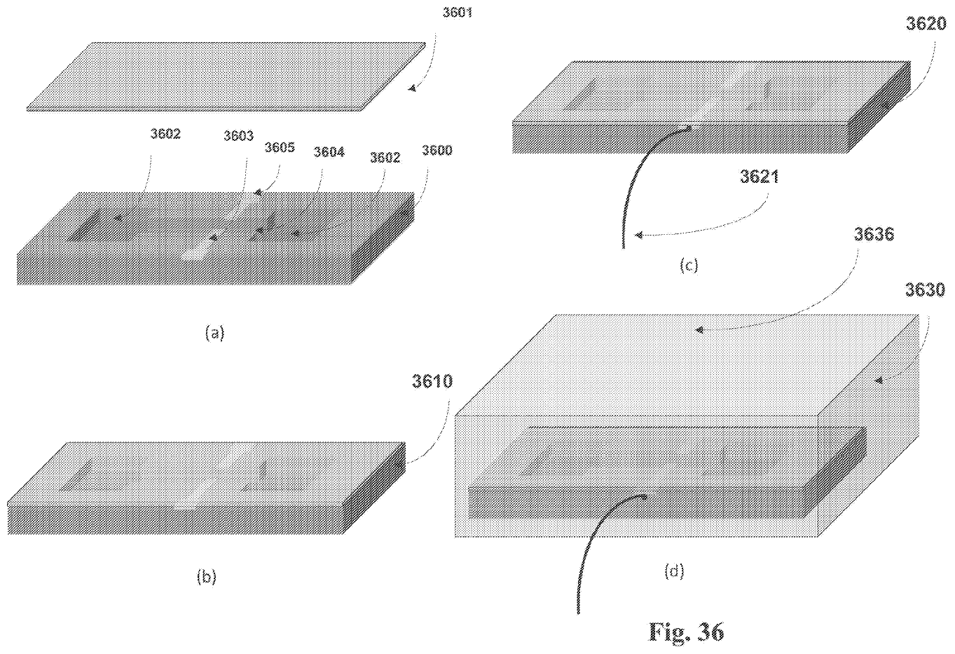

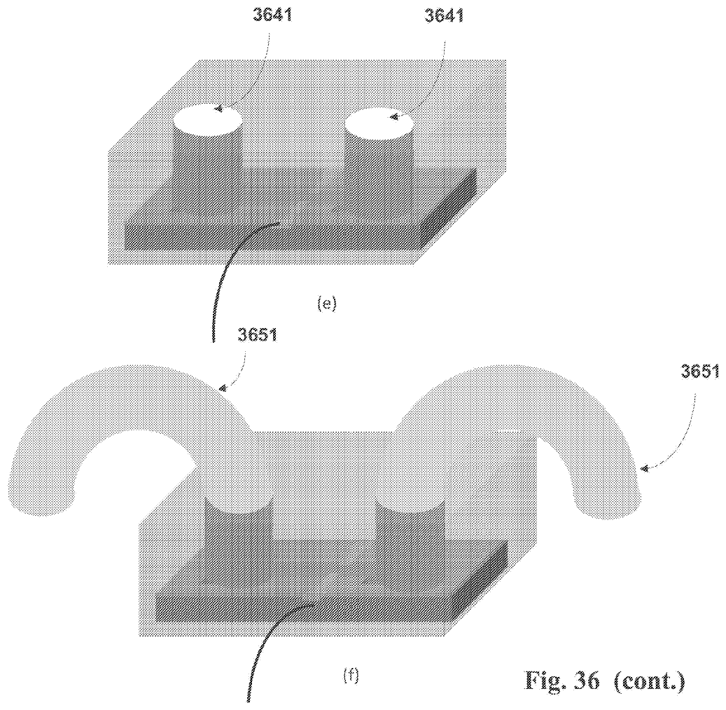

FIG. 36 shows another example of the apparatus of this invention packaged and ready for integration with a sample delivery system and data recording device. As shown in FIG. 36(a), a micro-device 3600 is fabricated by one or more micro-electronics processes as described in International Application No. PCT/US2011/042637, entitled “Apparatus for Disease Detection.” The micro-device 3600 has at least a micro-trench 3604, a probe 3603, a connecting port 3602, and a bonding pad 3605. On the top of the micro-device 3600, the surface layer comprises SixOyNz, Si, SixOy, SixNy, or a compound consisting of Si, O, and N. The surface layer can be covered, and thus the micro-device 3600 is mounted, with a flat glass panel 3601. See FIG. 36(b). The mounting can be by a chemical, thermal, physical, optical, acoustical, or electrical means. As shown in FIG. 36(c), the conductive wire is bonded with bonding pad from the side of the pads. FIG. 36(d) illustrates that the micro-device 3600 can then be packaged in a cube with only conducting wires exposed. The packaging cube can comprise a packaging material such as plastic, ceramic, metal, glass, or quartz. As shown in FIG. 36(e), a tunnel 3641 is then drilled into the cube until the tunnel reaches the connecting port 3602. Further, as shown in FIG. 36(f), the tunnel 3641 is then being connected to other pipes which can deliver a sample to be tested into the micro-device 3600, and flush out the sample after the sample is tested.

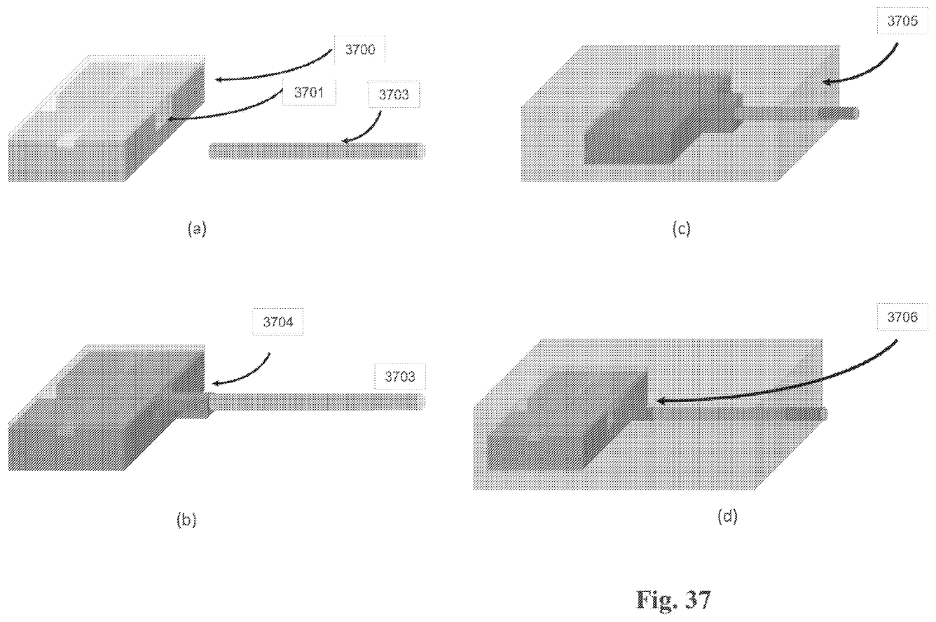

FIG. 37 shows yet another example of the apparatus of this invention packaged and ready for integration with a sample delivery system and data recording device. As illustrated in FIG. 37(a), device 3700 is a micro-fluidic device which has at least one micro-channel 3701. 3703 is a pipe that conducts a fluidic sample. The micro-channel 3701 and the conducting pipe 3703 are aligned and submerged in a liquid, for example, water. FIG. 37(b) illustrates that, when the temperature of the liquid in which the micro-device and conducting pipe are submerged, is decreased to its freezing point or lower, the liquid solidifies into a solid 3704. As illustrated in FIG. 37(c), while the temperature of the liquid is maintained below the freezing point, the combination (including the solid 3704, the conducting pipe 3703, and the device 3700) is enclosed into a packaging material 3705 whose melting temperature is higher than that of the solid 3704, with only the conducting pipe exposed. FIG. 37(d) shows that, after the temperature is increased above the melting point of the solid 3704, the solid material 3704 melts and becomes a liquid and is then exhausted from the conducting pipe 3703. The space 3706 wherein the solid material 3704 once filled is now available or empty, and the channel 3701 and the conducting pipe 3703 are now connected through and sealed in the space 3706.

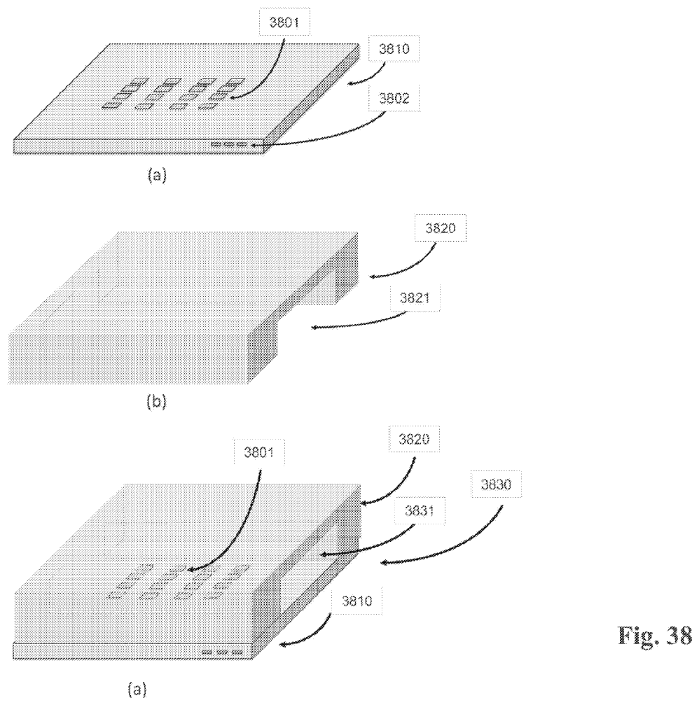

FIG. 38 shows an apparatus of this invention that has a channel (trench) and an array of micro sensors. In FIG. 38(a), 3810 is a device fabricated by microelectronics techniques; 3810 comprises micro-sensor array 3801 and addressing and read-out circuitry 3802. The micro-sensor array can include thermal sensors, piezo-electrical sensors, piezo-photronic sensors, piezo-optical electronic sensors, image sensors, optical sensors, radiation sensors, mechanical sensors, magnetic sensors, bio-sensors, chemical sensors, bio-chemical sensors, acoustic sensors, or a combination of them. Examples of thermal sensors include resistive temperature micro-sensors, micro-thermocouples, thermo-diodes and thermo-transistors, and SAW (surface acoustic wave) temperature sensor. Examples of image sensors include CCD (Charge Coupled Device) and CIS (CMOS image sensor). Examples of radiation sensors include photoconductive devices, photovoltaic devices, pyro-electrical devices, and micro-antennas. Examples of mechanical sensors include pressure micro-sensors, micro-accelerometers, micro-gyrometers, and micro flow-sensors. Examples of magnetic sensors include magneto-galvanic micro-sensors, magneto-resistive sensors, magneto diodes and magneto-transistors. Examples of biochemical sensors comprise conductimetric devices and potentiometric devices. FIG. 38(b) shows a micro-device 3820 that includes a micro-trench 3821. As illustrated in FIGS. 38(c), 3810 and 3820 are bonded together to form the new micro-device 3830 which include a trench or channel 3831. The micro-sensor array 3801 is exposed in the channel 3831.

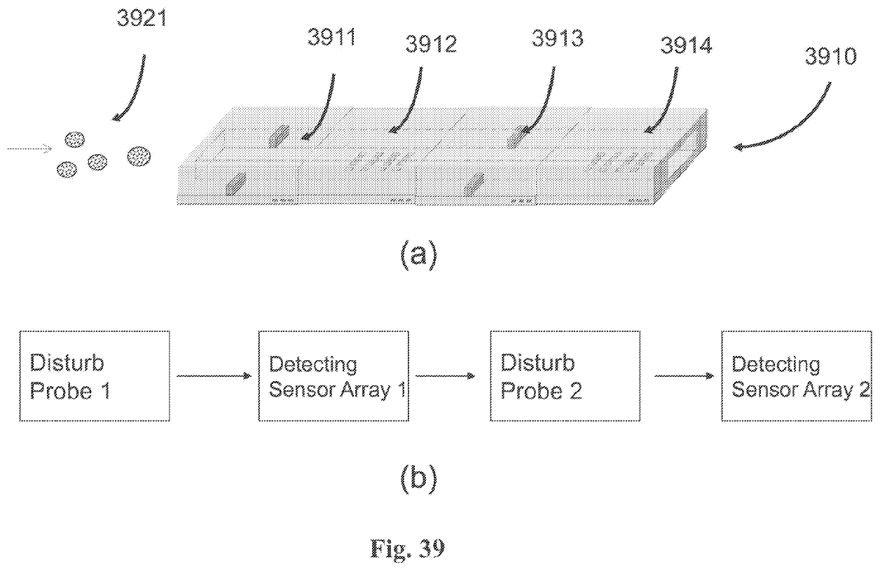

FIG. 39 shows another apparatus of this invention comprising several “sub-devices.” Particularly, as illustrated in FIG. 39(a), the device 3910 composes “sub-devices” 3911, 3912, 3913, and 3914, among which 3911 and 3913 are devices which can apply disturbing signals, and 3912 and 3914 are micro-sensor arrays. FIG. 39(b) illustrates the functioning diagram of the device 3910, when biological samples 3921 under the test are passing through the channel 3910, they are disturbed by signal A applied by 3911, then being tested and recorded by detecting sensor array 1 of 3912. These biological samples are then disturbed by disturb probe 3913 of array 2, and being tested by detecting sensor 3914 of array 2. Disturbing probe 3911 of array 1 and disturbing probe 3913 of array 2 can apply the same or different signals. Likewise, detecting sensor 3912 of array 1 and detecting sensor 3914 of array 2 can sense or detect the same or different properties.

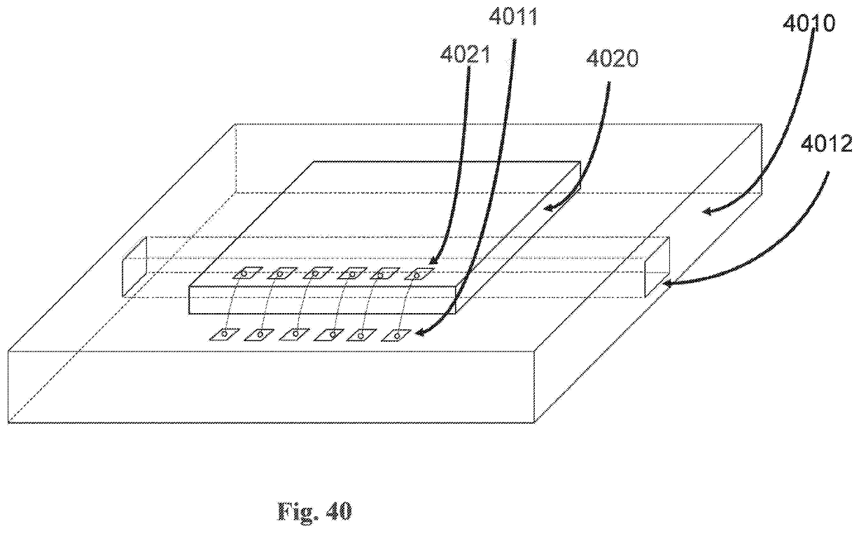

FIG. 40 shows an example of the apparatus of this invention which includes an application specific integrated circuit (ASIC) chip with I/O pads. Specifically, as illustrated in FIG. 40, 4010 is a micro-device with a micro-fluidic channel 4012 and I/O pads 4011. 4020 is an Application Specific Integrated Circuit (ASIC) chip with I/O pads 4021. 4020 and 4010 can be wired together through the bonding of I/O pads. As such, with an ASIC circuitry 4020, the micro-fluidic detecting device 4010 can perform more complicated computing and analytical functions.

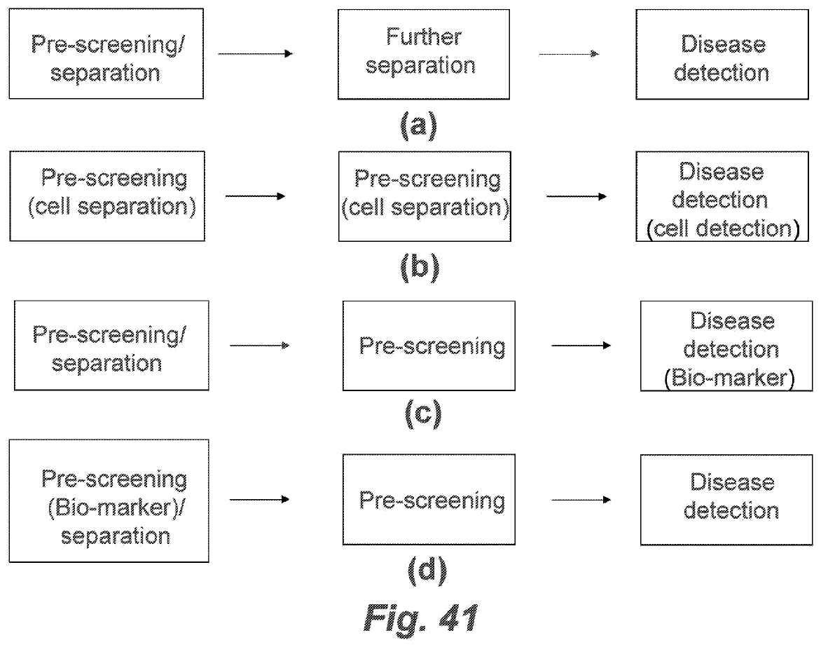

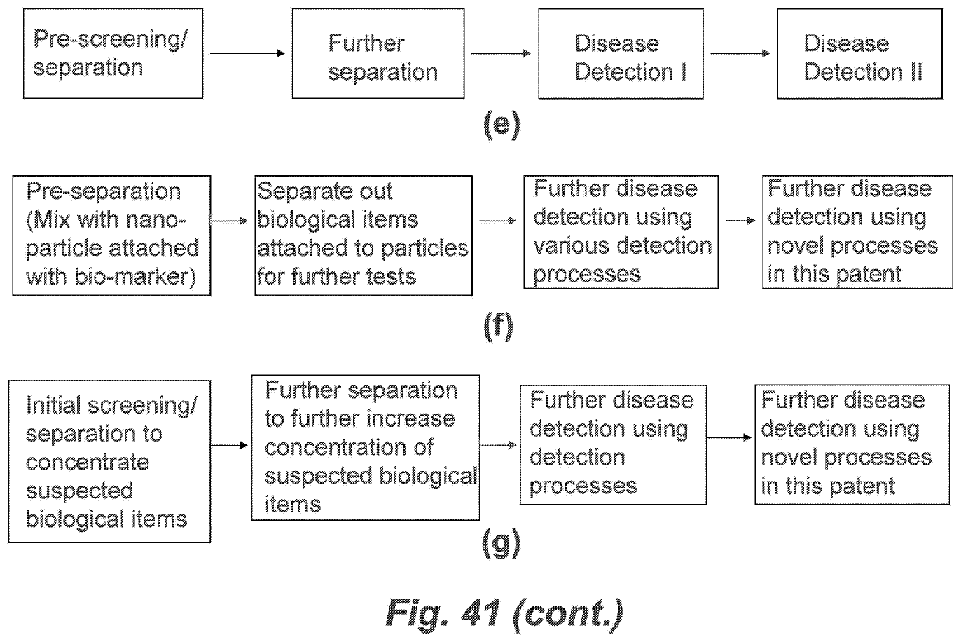

FIG. 41 is a diagram of the underlying principal of the apparatus of this invention which functions by combining various pre-screening and detection methods in unobvious ways. In FIG. 41(a), a biological subject is first pre-screened for diseased biological entities, and then the diseased biological entities are separated from the normal (healthy or non-diseased) biological entities. The biological subject containing the diseased biological entities separated from the normal biological entities is detected using a desired disease detection method. In FIG. 41(b), a biological sample has gone through multiple, successive cell separation steps to concentrate diseased cells (or biological entities). In FIG. 41(c), after pre-screening to concentrate diseased biological entities, bio-marker is used to detect diseased biological entities. In FIG. 41(d), bio-marker is first used to separate out diseased biological entities and then the sorted out, diseased biological entities are further detected by various detection methods. In short, this process includes initial screening, initial separation, further screening, further separation, probing with one or more disturbing signals or disturbing parameters (e.g., physical, mechanical, chemical, biological, bio-chemical, bio-physical, optical, thermal, acoustical, electrical, electro-mechanical, piezo-electrical, micro-electro-mechanical, or a combination thereof), and finally detection. This sequence can repeat one or more times. The effect of this process is concentrating the diseased entities for improved detection sensitivity and specificity, particularly for a biological subject with a very low concentration of diseased entities, such as circulating tumor cell (CTC).

In FIG. 41(e) through FIG. 41(g), a set of novel processes include (a) pre-screening, pre-separation and initial separation for diseased biological entities, (b) further separation of diseased biological entities, (c) optionally carry out initial detection, and (d) detection using various processes and detection methods. In the pre-separation process, one of the embodiments utilizes nano-particles or nano- magnetic particles attached with bio-markers to sort out diseased biological entities. During pre-separation process, the diseased biological entities are concentrated for higher concentration, which will make further separation and/or following detection easier. The biological sample following pre-separation process can go through further separation process to further enhance the concentration of diseased biological entities. Finally, the biological sample gone through the pre-separation and follow-up separation steps will go through detection step(s), in which various detection techniques and processes can be used to determine diseased biological entities and their types. In some embodiments, multiple detection steps can be utilized to detect diseased biological entities.

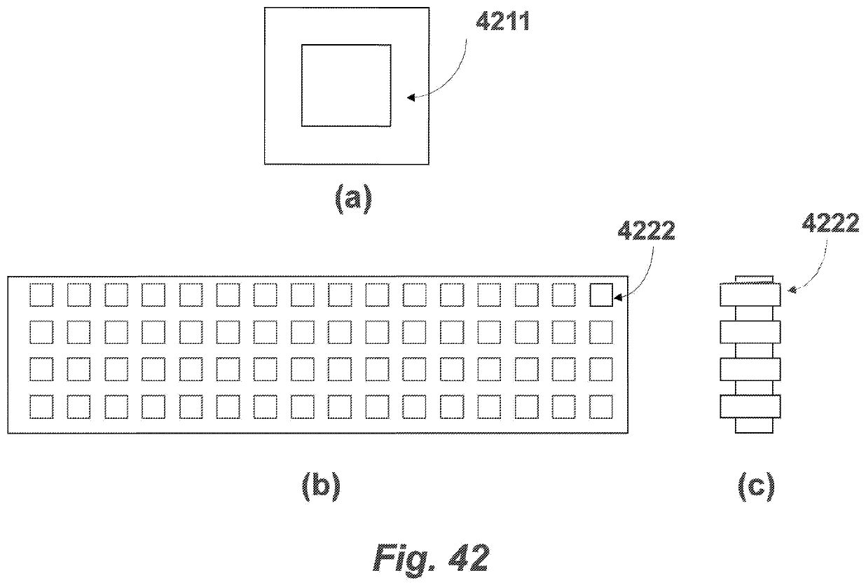

FIG. 42(a) shows a cross-sectional view of a channel (4211) into which a biological subject can flow. FIG. 42(b) shows an outside view of the channel, along which an array of detectors (4222) are installed along the path of the flow of the biological subject. Alternatively, both probes and detectors can be installed to both disturb the biological subject to be detected and detect response signals from such disturb signals. FIG. 42(c) shows a cross-section of the wall of the channel, where detectors (4222) are mounted through to contact the biological subject to be detected and also are making contact with the outside world (e.g., to connect to a detection circuitry).

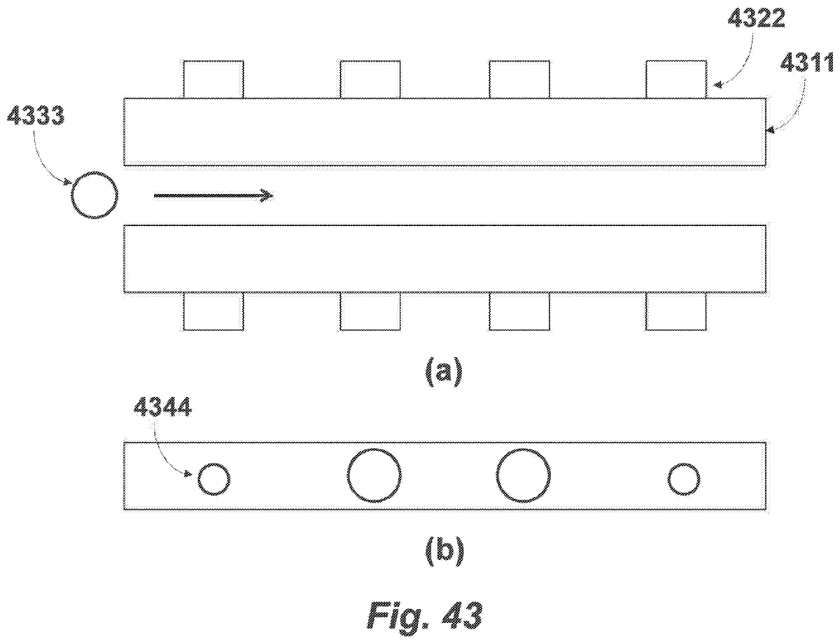

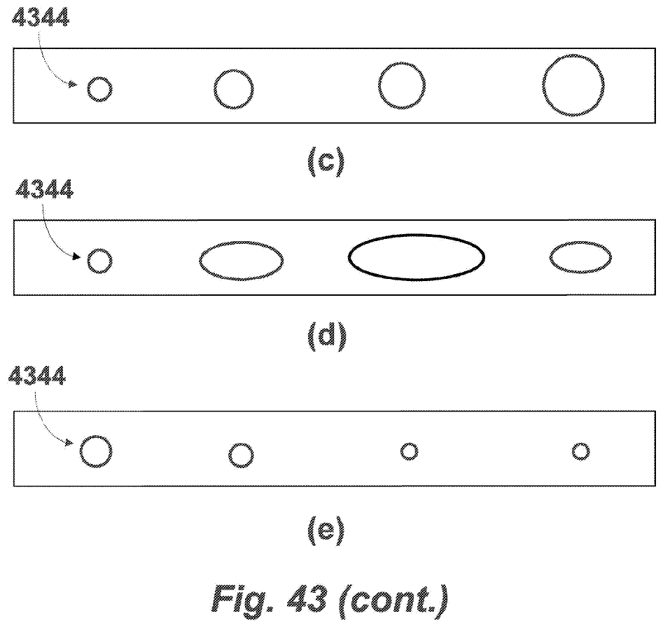

FIG. 43(a) shows a biological subject (4333) to be detected passing through a channel (4311) aligned with detectors (4322) along its passage. The detectors can be the same type of detectors, or a combination of various detectors. Further, probes capable of sending out probing or disturbing signals to the biological subject to be detected can also be implemented along the channels, along with detectors which can detect response from the biological subject which has been probed or disturbed by the probe. The detected signals can be acoustical, electrical, optical (e.g., imaging), biological, bio-chemical, bio-physical, mechanical, bio-mechanical, electro-magnetic, electro-mechanical, electro-chemical-mechanical, electro-chemical-physical, thermal, and thermal-mechanical property related signals, or a combination of them. FIG. 43(b) shows an example of a set of detected signals (e.g., images, pressures, or electrical voltages) (4344) along the path of the biological subject, which recorded its behavior and properties as it passes through the channel. For example, for an optical detector, the size of the circle shown in the FIG. 43(b) could mean the optical emission from the biological subject (such as an optical emission from a florescence component attached to the biological subject), the strength of a strain (pressure) acting on the side wall of the channel detected by a piezo-electric detector or a piezo-photronic detector, or thermal emission from the biological subject detected by a thermal detector or an IR sensor. Such detected signals can be solely from the biological subject as it passes through the channel, or responses from the biological subject to a disturbing or probing signal by the probe.

Like FIG. 43(b), FIG. 43(c) through FIG. 43(e) show additional examples of various detected signal patterns (4344) as the biological subject passes through the channel and is detected by the novel detectors and processes disclosed in the application.

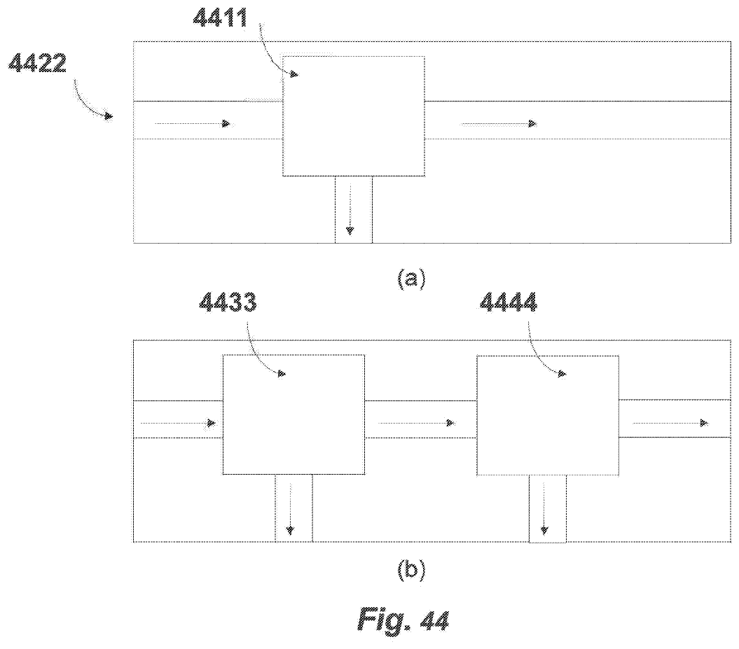

To effectively sorting, separating, screening, probing, or detecting of diseased biological entities, a chamber (or chambers) integrated with various channels can be deployed as shown FIG. 44(a), where incoming sample flowing into a chamber (4411) first. In the chamber, various techniques such as bio-markers and nano-technology (magnetic beads or nano-particles with bio-markers attached to them) based processes can be used to sort out, screen, and separate out the diseased biological entities. For example, a biological sample flowing from the left into the chamber can have its diseased entities separated out in the chamber, and passed downward through the bottom channel, while its normal entities can continue to flow from the chamber in the right hand direction, through the channel in the right side of the chamber. Depending upon the design, the diseased entities, having entered into the chamber on the left, can also be separated out in the chamber, and continue on towards right and flow into the channel on the right side of the chamber, while normal entities will continue to flow down toward and through the channel at the bottom of the chamber. FIG. 44(b) shows multiple chambers integrated with channels in which biological entities can be sorted, screened, separated, probed or detected. In the application of screening and separation, the multiple chambers can carry out multiple screening and separation steps. As shown in FIG. 44(b), for a biological sample flowing from the left toward the right direction, it will enter into the first chamber on the left (4433) and undergo a first screening and separation. The biological sample can continue to flow towards the right, enter into the second chamber, the chamber on the right (4444), and undergo a second screening and further separation. In this way, through a multi-staged screening and separation process, the concentration of a diseased entity can be successively enhanced which can be helpful for a sensitive final or late stage detection. This type of device design and process could be very useful for defection of a biological sample with an initially very low concentration of diseased entity population, such as for the detection of circulating tumor cell (CTC) which is typically in the concentration of one part in one billion cells or 10 billion cells.

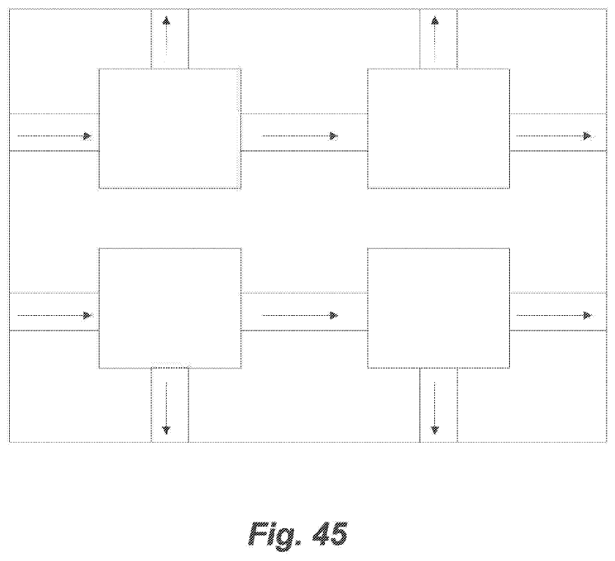

To significantly speed up the sorting, screening, probing and detection operations using the disclosed device and process, a high number of desired structures such as those discussed in FIG. 45 can be fabricated simultaneously on the same chip as shown in FIG. 45.

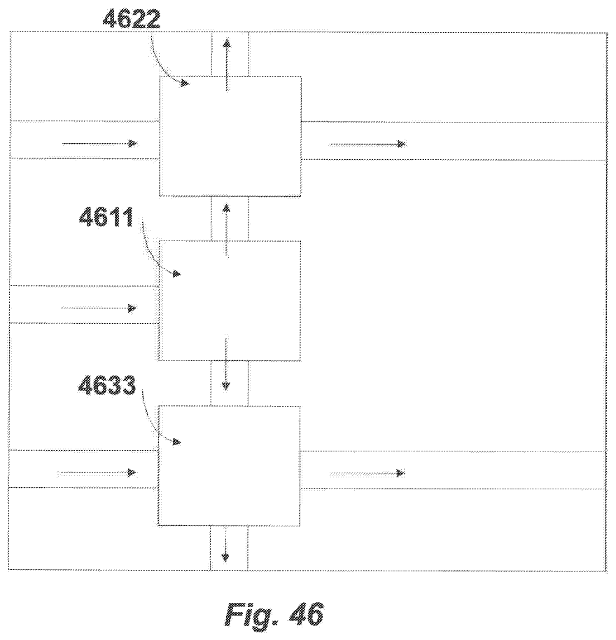

FIG. 46 shows another novel device layout for sorting, screening, separating, probing and detecting diseased biological entities, in which a desired component or multiple components through the middle channel into the middle chamber 4611 can play a wide range of roles. For example, the component flowing into the middle chamber could be a bio-marker which can be freshly added into the top chamber 4622 and bottom chamber 4633 when its (bio-marker) concentration needs to be adjusted. The timing, flow rate, and amount of component in the middle chamber 4611 need to be added into the top and bottom chambers (4622 and 4633) can be pre-programmed or controlled via a computer or software in real time. The component into the middle chamber 4612 could also be nano-particles or magnetic beads attached to bio-markers. In another novel embodiment, the component into the middle chamber 4611 could be a disturbing agent which will disturb the biological subject or samples to be detected in the top and bottom chambers.

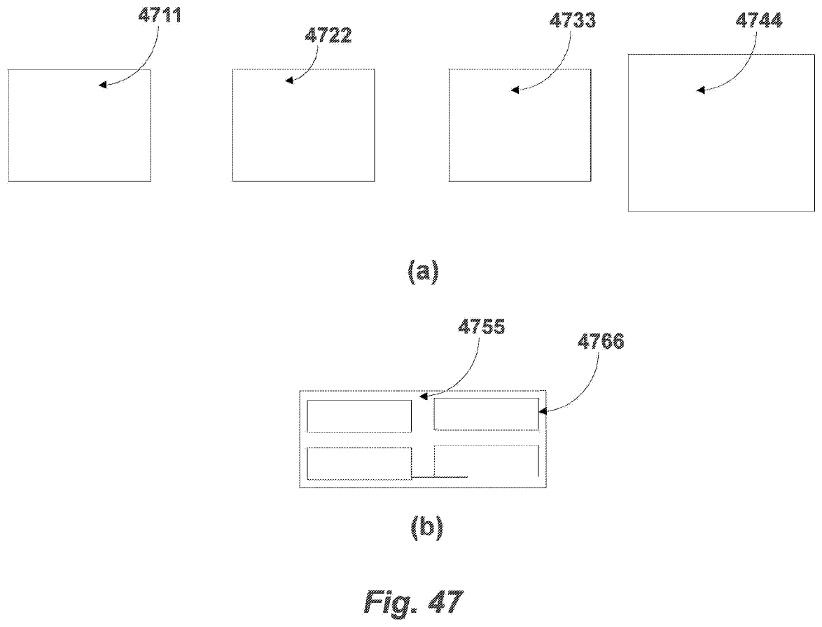

FIG. 47 shows that, compared with multiple standalone detection apparatuses (see FIG. 47(a), 4711, 4722, 4733, and 4744), an apparatus (4755) with multiple sub-units of different functions and technologies (4766) assembled or integrated has a significantly reduced apparatus volume or size (see FIG. 47(b)), therefore reduced costs since many common hardware (e.g., a sample handling unit, a sample measurement unit, a data analysis unit, a display, a printer, etc.) can be shared in an integrated apparatus. For example, such a multi-functional, integrated apparatus can include a bio-marker detector, an imaging based detector, a photo-detector, an x-ray detector, a nuclear magnetic resonance imaging detector, an electrical detector, and an acoustic detector all of which are assembled and integrated into the single apparatus, so that the apparatus can have improved detection functionality, sensitivity, detection versatility, and reduced volume and cost.

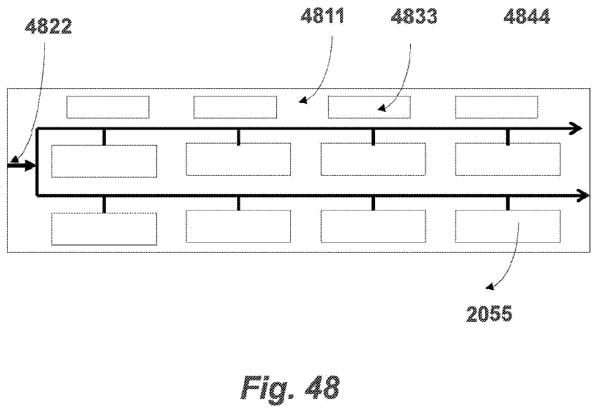

FIG. 48 shows that when multiple sub-units with different functions and technologies (2055) are assembled into one apparatus, a more diverse functionality, improved detection functionality, sensitivity, detection versatility, and reduced volume and cost can be achieved, where a number of common utilities including, e.g., input hardware, output hardware, sample handling unit, sample measurement unit, data analysis unit and data display unit (4811, 4833, and 4844) can be shared. For example, when a range of detection units utilizing various detection technologies are assembled into one apparatus, many functions and hardware such as sample handling unit, sample measurement unit, data transmission unit, data analysis unit, computer, and display unit can be shared, thereby significantly reducing the apparatus' equipment volume or size, costs, and complexity while improving measurement functionality and sensitivity.

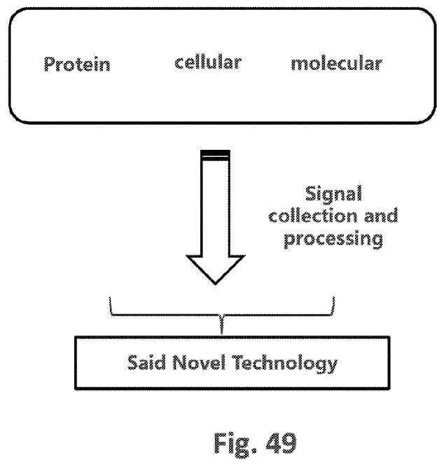

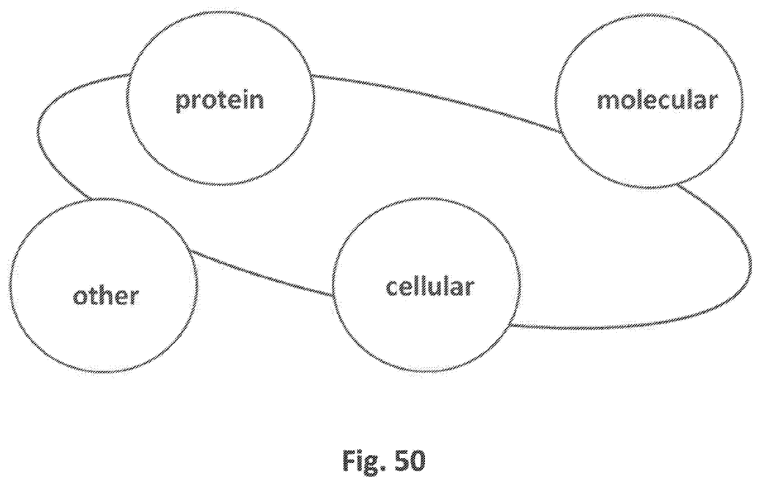

One of the key aspects of the present invention relates to a novel technology for detecting disease, in which a number of different classifications of biological information are collected in a device and processed or analyzed. For instance, FIG. 49 shows a number of different classifications of biological information (e.g., protein, cellular, and/or molecular) can be collected in a device according to the present invention, and processed in the novel technology according. As shown in FIG. 50, the measured information according to the present invention includes protein, cellular and molecular level information, or combination of them.

Tests were carried out in the laboratory with the apparatus of this invention on certain cancerous tissue samples (with multiple samples for each type of cancer) although the apparatus of this invention can be used for detection of other types of cancer or other types of treatment. In the tests, healthy control samples were obtained from animals with no known cancer disease at the time of collection and no history of malignant disease. Both cancerous samples and healthy control samples were collected and cultured in the same type of culture solution. The cultured samples were then mixed with a dilution buffer and diluted to the same concentration. The diluted samples were maintained at the room temperature for different time intervals and processed within a maximum of 6 hours after being recovered. The diluted samples were tested at the room temperature (20˜23 oC) and in the humidity of 30%˜40%. The samples were tested with an apparatus of this invention under the same conditions and stimulated by the same pulse signal.

The tests show that, in general, the control groups' tested (measured) values (i.e., measured values in relative units for the testing parameter) were lower than the cancerous or diseased groups. Under the same stimulation (in terms of stimulation type and level) with a stimulating or probing signal applied by a probing unit of the tested apparatus of this invention, the difference shown in the measured values between the control groups and the cancerous groups became much more significant, e.g., ranging from 1.5 times to almost 8 times in terms of level of increase in such difference, compared with that without simulation. In other words, the cancerous groups' response to the stimulating signal was much higher than that of the control groups. Thus, the apparatus of this invention has been proven to be able to significantly enhance the relative sensitivity and specificity in the detection and measurement of diseased cells, in comparison to the control or healthy cells.

Further, the test results show that in terms of the novel parameter utilized by the apparatus of this invention, the cancerous group and the control group showed significantly different response. Such difference is significantly greater than the measurement noise. There was a large window to separate the control groups from the cancerous groups, showing a high degree of sensitivity of the novel measurement method and apparatus.

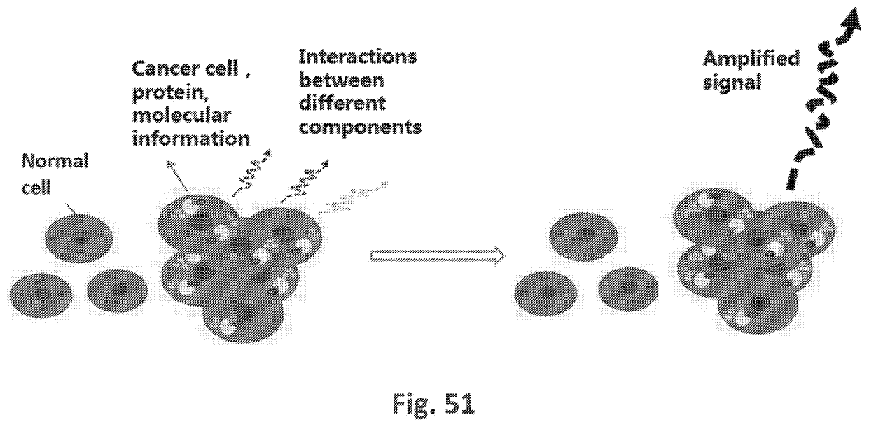

FIG. 51 shows signals from different biological classifications may interact, combine, and/or amplify to enhance signal in this novel technology. Compared with the traditional technology, signal and information collected by the apparatus and methods of this invention is linearly and can even be non-linearly amplified; and additional two-factor and three-factor (or higher order) interactions between various levels (cellular, protein, molecular or other levels) and components/parameters (exemplified in the following table) are not only just novel, unique, but also exhibited unexpected reliable and sensitive results when compared to the traditional technology.

| | | This |
|---|---|---|
| Traditional technology | | invention |
| P - protein based (bio-marker, AFP, CEA, PSA, etc.) | | P |
| C - cellular based (CTC, ctDNA) | + | C |
| M - Molecular (genomics, DNA, RNA) | + | M |
| => one-dimensional information | + | P-C |
| | + | P-M |
| | + | M-C |
| | + | M-C-P |
| | => seven | |
| | dimensional info | |
| Other level/parameter (O) | + | M-C-P-O |
| | => more | |
| | dimensions info | |

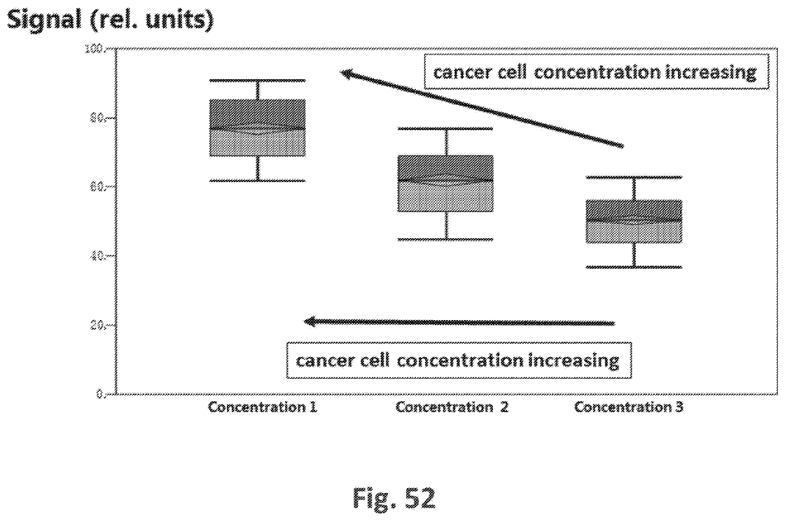

FIG. 52 shows detected signal in this novel technology as a function of cancer cell concentration. The results provided in FIG. 52 show that the signal increases with increasing amount of cancer cells.

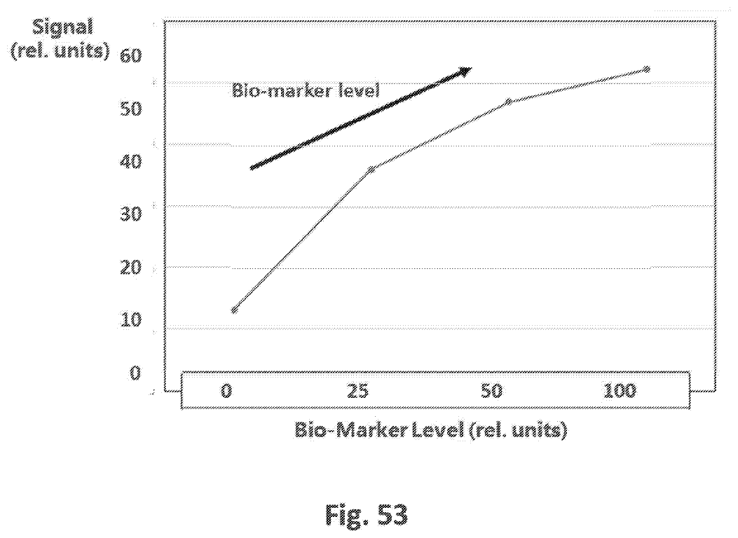

FIG. 53 shows detected signal in this novel technology as a function of a bio-marker level. The results provided in FIG. 52 show that the signal increases with increasing level of bio-marker.

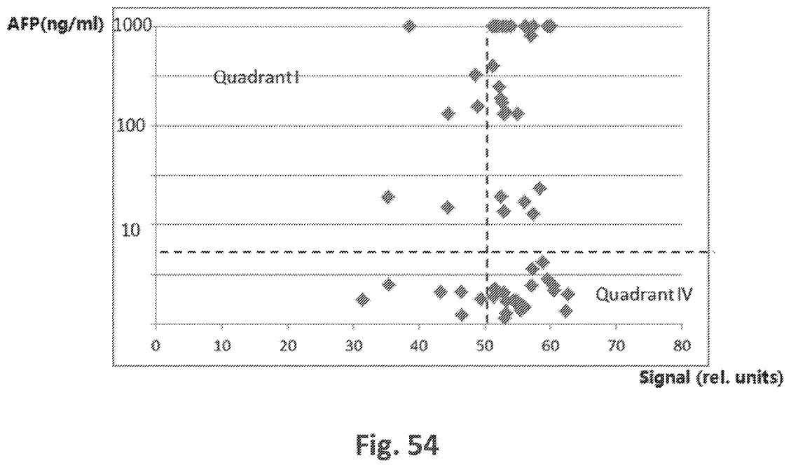

FIG. 54 shows test results proving an advantage of this novel technology compared with traditional bio-marker (AFP) for liver cancer. As shown in FIG. 54, using 58 confirmed liver cancer samples, sensitivity of this novel technology is 79.3%, which is significantly higher than that of AFP (i.e., 55.9%).

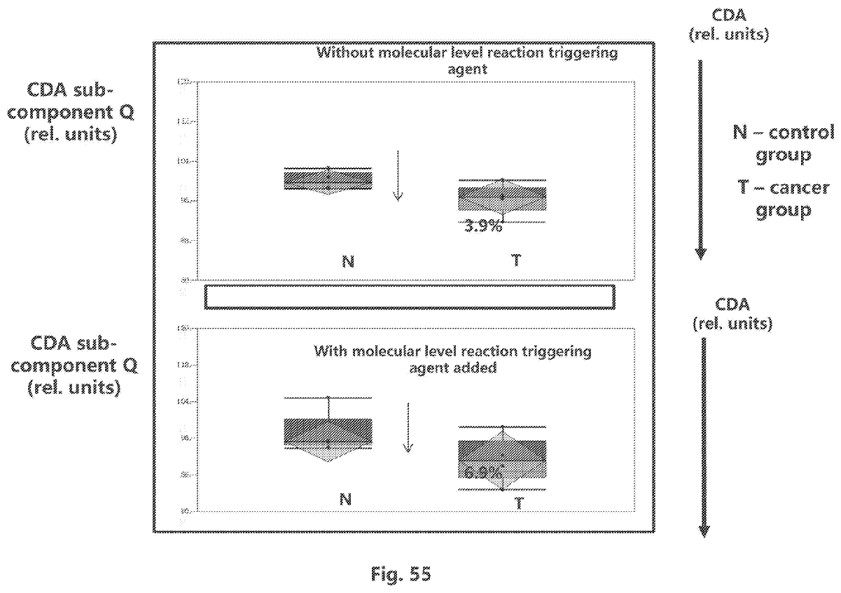

Studies were also undertaken to examine the effect of adding molecular level reaction triggering agent on the efficacy of the apparatus and methods for detecting disease of this invention. The results provided in FIG. 55 show that the difference in signal between the control (healthy) group and cancer group was increased, indicating the detection system did detect molecular level information.

The apparatus and methods of this invention has been used in test of more than 20 different types of cancer in all stages of development and showed expectedly high sensitivity and specificity. FIG. 56 shows that to validate the usefulness and sensitivity of this invention, over 60,000 samples were collected, with 30,000 samples in retrospective investigation, and 30,000 samples in general screening, and remarkable sensitivity and selectivity of this invention was demonstrated from testing those samples.

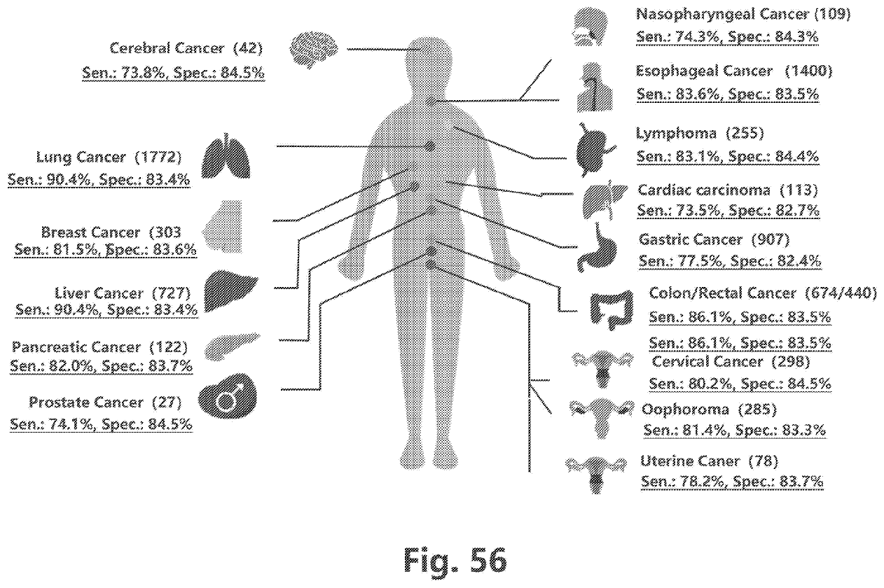

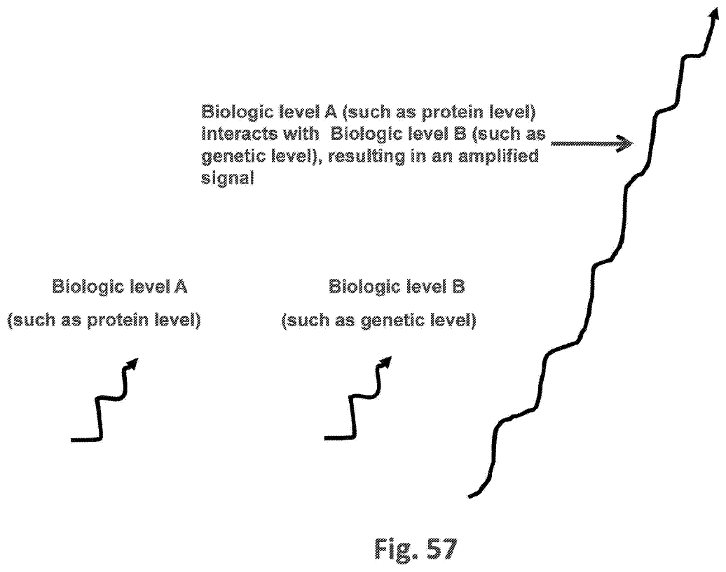

FIG. 57 shows in a multi-level detection system of this invention, one biological level (for example, protein) can interact with another biological level(s) (such as genetic level), resulting in synergistic reactions and resultant amplification in signal.

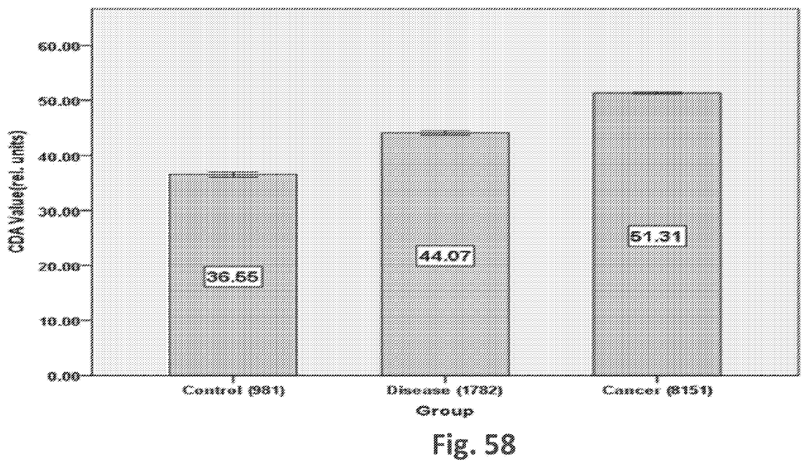

FIG. 58 shows the CDA values of the control group, non-cancer disease group and cancer group. As detected by the apparatus and methods of this invention, the cancer group always has a higher CDA value than that of a non-cancer disease group, and this difference in CDA value between the cancer group and non-cancer disease group is statistically significant particularly for monitoring the progression of a disease state, e.g., from an inflammatory disease to a pre-cancer condition to a malignant cancer or tumor and then to a late stage cancer. In other words, CDA values can be used in a disease and cancer-differentiating analysis with the help of the apparatus and methods of this invention.

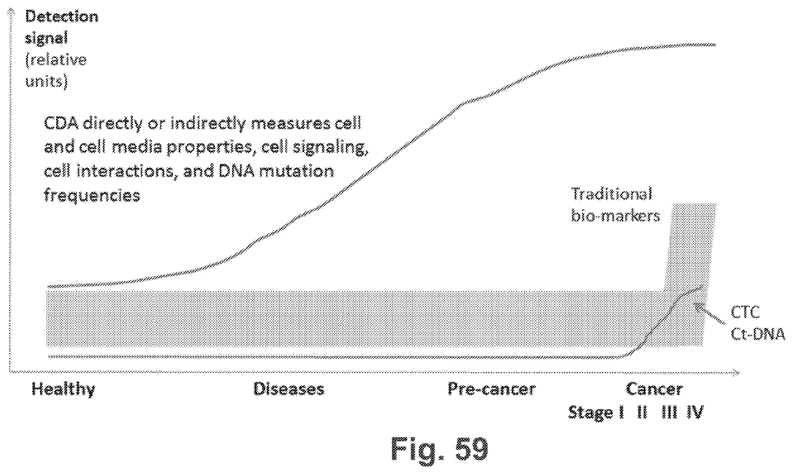

FIG. 59 shows the relationship between disease state and detected cell signaling properties and/or cell media properties. Traditional cancer screening and prognosis IVD methods such as bio-markers and genomics (e.g., circuiting tumor-DNA (ct-DNA)) are unable to detect cancer early, and have relatively lower signals. Bio-markers are not effective for early stage cancer detection (as shown in FIG. 59), but also lack markers for a number of cancer types. In the case of CTC and ct-DNA, as also shown in FIG. 59, signals occur only after solid tumor has been formed, making early stage cancer detection relatively. Compared with those traditional methods, the novel CDA technology according to the present invention can directly or indirectly measures cell and cell media properties, cell signaling, cell interactions, and/or DNA mutation frequencies, thereby resulting in significantly higher signals, which are available for even pre-cancer or early stage cancer detections.

Another major novel aspect of this application relates to an effective method to probe and track ability (including immune system) to detect and prevent potential diseases, ability to flight diseases, and the state of a life body, including but not limited to healthy state, non-cancer disease state, pre-cancer state, and cancer state.

Using a novel microfluidic device equipped with sensitive sensors and a fully automated testing machine developed in this work, the method of this invention has been demonstrated on about 100,000 samples which included control (healthy group), disease group, pre-cancer disease group, and cancer group individuals. The test results showed statistically significant blood micro-electrical current level decreasing from healthy group to disease group, and further decreasing to cancer group, signifying potential importance of this new detection technology for early stage cancer detection. In early stage non-small cell lung cancer (NSCLC) tests, sensitivity and specificity reached ˜85% and 93%, respectively. It has also shown that it is capable to detect over 20 types of cancer, including esophageal cancer and brain tumor which do not have other effective screening methods. As the class of electrical properties is a fundamental bio-physical sub-field and impacting many aspects of human blood, it has multi-level effects at cellular, protein, and even molecular levels. Data appear to reveal that this novel technology provides a potentially powerful insight into how cancer evolves and can be highly valuable for pre-cancer and early stage cancer detection. Its mechanism, potential significance, and ramifications will be presented.

Since the liquid media (for example, blood) is interfacing, connecting and communicating with both cells, proteins, and genetic components (DNAs, RNAs, etc.), it plays a critical role in the interfacing, interactions, and communications (for example, cell signaling) between cells, proteins, and genetic components (DNAs, RNAs, etc.) and other biological entities, and the occurrence and progression of diseases including but not limited to non-cancer diseases, pre-cancer diseases and cancer. On the other hand, in the transition from a healthy individual to a disease state, immune system is degraded and disease detection and killing agents such as T cell lost function. In this invention, it is believed that immune system degradation (decrease) and loss in disease detection and disease fighting will and action is caused by changes in properties in the said liquid media surrounding cells, proteins, genetic components (DNAs, RNAs, etc.) and other biological entities. Specifically, those properties can be biological properties (protein concentration, protein types, DNA sequence, DNA static electrical force, DNA surface charge, DNA surrounding media electrical properties, quantum mechanical effects, etc.), bio-chemistry properties, physical properties (thermal, mechanical, electrical, and electro-magnetic properties), bio-physical properties, properties. For example, the shift in the above property (for example, reduction in the above said physical properties) may affect (for example, reduction in effectiveness and efficiency, and transduction degradation) cell signaling and communications by cells and between cells and other biological entities, resulting in the compromise of immune system, loss of detection capability of cells such as T cells to detect cancer cells and ability to kill cancer cells. Therefore, by measuring the above properties including physical and bio-physical properties, one is able to detect the onset of disease and track disease from one stage to the next stage, making early detection and prevention of disease possible.

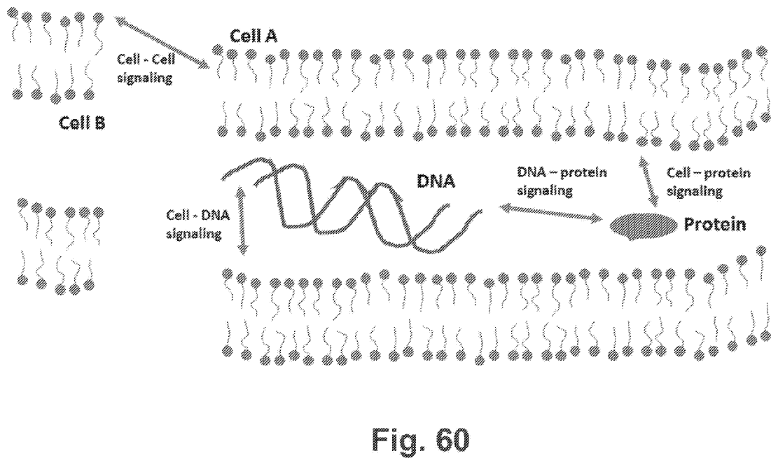

FIG. 60 shows that in a blood sample, among other components, there are cells, proteins, and genetic components (DNAs, RNAs, etc.) which are surrounded by a liquid media interacting with the just-mentioend components. In addition, cell interacts and communicates (for example, one cell through its surface signaling interacts and communicates with the surface of another cell via acoustical, optical, electro-magnetic and electrical means) with other cells and other biological entities including but not limited to proteins and genetic components (DNA, RNA, etc.) via cell signaling. At the same time, proteins and genetic components (DNAs, RNAs, etc.) can interact with other protein components and genetic components (DNAs, RNAs, etc.). Since the liquid media around which the cells, proteins, and genetic components (DNAs, RNAs, etc.) are within interacts and interfaces with all the above said biological entities, the media plays a critical role and function in signal transmission, interactions, and functions of the above said biological components which may (a) affect healthy or disease states of the biological body, (b) progression of diseases such as non-cancer diseases, pre-cancer diseases, and cancer, and (c) ability for diseases such as cancer to evade/escape detection and/or elimination by immune system and/or disease killing agents such as T cells. Through measuring physical, bio-physical, chemical, biological, and bio-chemical properties of the said media and cell signaling, one is expected to be able to detect and track immune system, resistance to diseases, ability to detect diseases, ability to flight diseases, and the state of life body, including but not limited to healthy state, non-cancer disease state, pre-cancer state, and cancer state. The above said physical properties include but not limited to acoustical, optical, mechanical, chemical, bio-chemical, electrical, electro-magnetic, and thermal properties.

### Exemplary Test

Mechanism

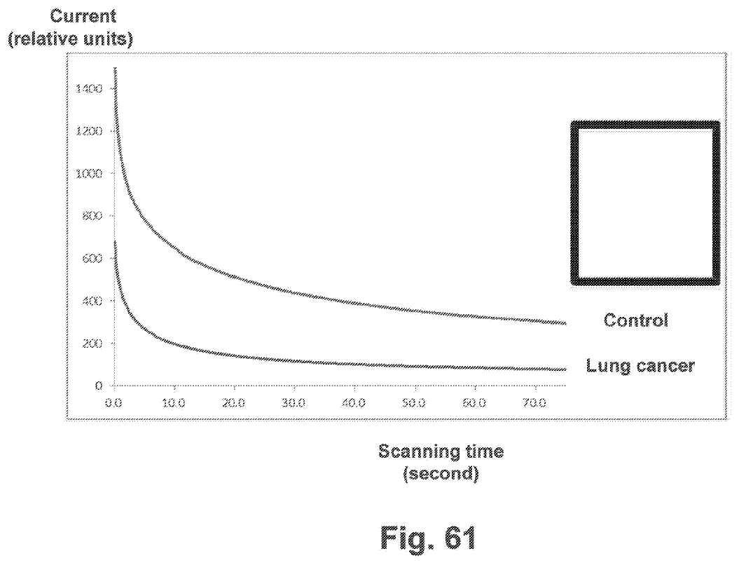

A micro fluidic device was fabricated by an integrated circuit method in which micro-channels were formed along which sample fluid can be passed, and on whose sides detection transducers (i.e., sensors) were formed to probe the fluid. During dada collection, a voltage meter with automated data recording capabilities was used. When fluid sample arrives at a micro-channel, sensors in the channel can probe the sample via applying a constant voltage while recording micro- electrical current response as a function of time dependent behavior (time sweep) as shown FIG. 61 for control (healthy) and cancer cell line samples, in which a typical micro- electrical current curve is shown, with Y axis being current and X axis being time. The characteristic current versus time curve collected is dependent upon the properties of the samples measured and reveals the state of the individual tested. Fully automated test machine consisting of sample transport units, mixing chamber, and testing unit with micro-fluidic device is designed and assembled for data collection.

Cell Line Characteristics

Four cell lines were utilized in the preliminary research. Human non-small cell lung cancer cell line A-549 (Cat. No. TCHu150), human embryonic lung cell line MRC-5 (Cat. No. GNHu41), human hepatoma cell line QGY (Cat. No. TCHu 42) and human hepatocyte cell line HL-7702 (Cat. No. GNHu 6), which were purchased from Cell Bank of Typical Culture Preservation Committee of Chinese Academy of Sciences/Cell Resource Center of Shanghai Academy of Life Sciences, Chinese Academy of Sciences, were cultured in complete growth medium of RPMI-1640 medium which contain 10% FBS (fetal bovine serum) and 1% penicillin-streptomycin in atmosphere of 95% air and 5% carbon dioxide in 37° C. Cell suspension solutions were prepared for testing.

Blood Sample Characteristics

Samples used in a CDA test were whole blood or serum samples, with whole blood typically used.

Whole blood was drawn into an EDTA tube with anticoagulant agent. In addition, cell lines for both control (healthy) and cancer samples were also used in initial development phase of the work to test and validate signals of the technology.

Algorithm

With a large data base from retrospective studies, an algorithm has been built with a CVD test numbers along with cut-off values as a test outcome which is correlated to cancer risk, which (CDA value) is proportional to cancer risk. Based on CDA values, three regions were divided, healthy, medium risk, and high risk.

Results

Both retrospective studies and population screenings were carried out. Follow-up was carried out on a group of individuals with low, medium, and high risk values, among which feedback on 5809 individuals was obtained.

FIG. 61 shows scanning curves of control (healthy) and lung cancer cell lines, indicating that the electronic current for lung cancer is much lower than that of the control group. Specifically, it shows a typical curve for a control cell line sample (healthy cell line) and lung cancer cell line, with electrical current decreasing overtime and reaching a stable value in both cases. The two curves showed clearly different values at multiple points on the curves, especially significant difference in electrical current values between the two curves at their respective resting positions (60 seconds), indicating that this novel technology could distinguish normal cells and cancerous cells.

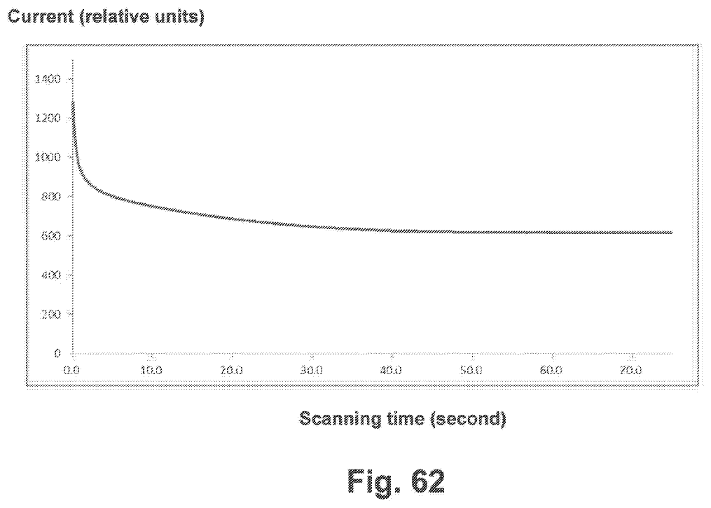

Furthermore, there is noticeable difference between control, disease and liver cancer samples (FIGS. 62-64), with decreasing electrical current from control state to disease state, and from disease state to cancer state, demonstrating potential viability of this novel approach to detect disease and cancer, and ability to track disease progression.

FIG. 62 shows a typical scanning curve for control (healthy) whole blood sample, indicating a similar profile as that for a control cell line sample.

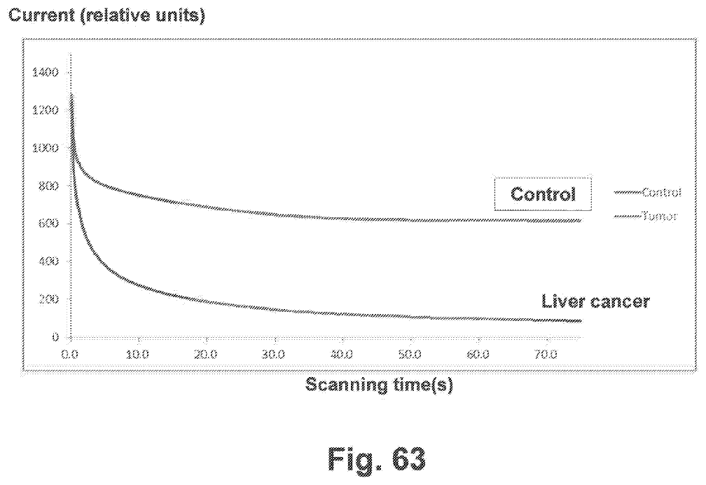

Data for a typical control whole blood sample and a liver cancer whole blood sample are shown in FIG. 63, showing again ability to differentiate a normal sample from a cancer sample.

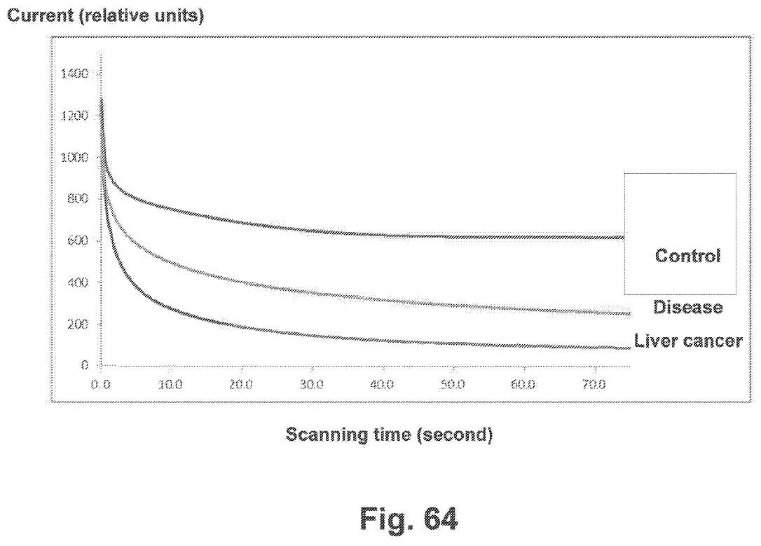

FIG. 64 are a set of scan traces for whole blood samples of control, disease and liver cancer. FIG. 64 shows noticeable difference between control, disease and liver cancer samples, with decreasing electrical current from control state to disease state, and from disease state to cancer state, demonstrating potential viability of this novel approach to detect disease and cancer, and ability to track disease progression.

Having initially confirmed feasibility of this new technology for disease detection, multiple retrospective clinical studies have been carried out. Data on over 20 types of cancer have been collected, and an algorithm has been built based upon a large data base. A set of test parameters have been built around the above-mentioned algorithm. The key parameter calculated from this algorithm based on raw data is CDA indicator, whose value is proportional to the cancer risk, and inversely proportional micro- electrical current value of the sample tested.

Table 8 shows significance test of difference—non-parametric test of various types of cancer. In Table 8, the distribution of CDA is the same across the categories of Group. Asymptotic significances are displayed. The significance level is 0.05. Table 8 shows that the difference in CDA values between control group and various cancer types are of statistical significance.

TABLE 8
Hypothesis Test Summary

| Null Hypothesis | Test | Sig. | Decision |
|---|---|---|---|
| Control (1717) vs. | Independent | 0.000 | Reject the |
| Cancer (10078) | Samples | | null Hypothesis |
| Control (1717) vs. | Mann Whitney | 0.000 | Reject the |
| Lung Cancer (1907) | U Test | | null Hypothesis |
| Control (1717) vs. | | 0.000 | Reject the |
| Colon Cancer (710) | | | null Hypothesis |
| Control (1717) vs. | | 0.000 | Reject the |
| Esophageal Cancer (1590) | | | null Hypothesis |
| Control (1717) vs. | | 0.000 | Reject the |
| Gastric Cancer (1117) | | | null Hypothesis |
| Control (1717) vs. | | 0.000 | Reject the |
| Rectal Cancer (522) | | | null Hypothesis |
| Control (1717) vs. | | 0.000 | Reject the |
| Cardia Cancer (135) | | | null Hypothesis |
| Control (1717) vs. | | 0.000 | Reject the |
| Liver Cancer (738) | | | null Hypothesis |
| Control (1717) vs. | | 0.000 | Reject the |
| Pancreatic Cancer (134) | | | null Hypothesis |
| Control (1717) vs. | | 0.000 | Reject the |
| Ovarian Cancer (337) | | | null Hypothesis |
| Control (1717) vs. | | 0.000 | Reject the |
| Breast Cancer (348) | | | null Hypothesis |
| Control (1717) vs. | | 0.000 | Reject the |
| Cervical Cancer (318) | | | null Hypothesis |
| Control (1717) vs. | | 0.000 | Reject the |
| Uterine Cancer (105) | | | null Hypothesis |
| Control (1717) vs. | | 0.000 | Reject the |
| Prostatic Cancer (31) | | | null Hypothesis |
| Control (1717) vs. | | 0.000 | Reject the |
| Brain Tumor (50) | | | null Hypothesis |
| Control (1717) vs. | | 0.000 | Reject the |
| Lymphoma (322) | | | null Hypothesis |
| Control (1717) vs. | | 0.000 | Reject the |
| Nasopharyngeal | | | null Hypothesis |
| Cancer (121) | | | |
| Control (1717) vs. | | 0.000 | Reject the |
| Other Cancer (1593) | | | null Hypothesis |

A summary of cancer screening sensitivity and specificity for control group and a number of cancer types from retrospective study is given in Table 9. Table 9 showed that overall, both sensitivity and specificity of CDA technology of various cancer types are relatively high, demonstrating CDA technology is potentially suited for a large number of cancer types. In addition, statistical analysis of the data Table 8 showed that P values for each two groups (each cancer group and control group) are all less than 0.001, also meaning that the difference in CDA values between control group and various cancer types listed in Table 8 are of statistical significance.

TABLE 9
CDA technology demonstrates high sensitivity and specificity
for cancer screening of various types of cancer

| Control (1717) vs. | Sensitivity | Specificity |
|---|---|---|
| Cancer (10078) | 86.6% | 86.9% |
| Lung Cancer (1907) | 88.4% | 88.4% |
| Colon Cancer (710) | 87.7% | 87.4% |
| Esophageal Cancer (1590) | 86.9% | 86.8% |
| Gastric Cancer (1117) | 82.4% | 86.8% |
| Rectal Cancer (522) | 83.1% | 86.8% |
| Cardia Cancer (135) | 79.3% | 87.0% |
| Liver Cancer (738) | 89.7% | 88.8% |
| Pancreatic Cancer (134) | 82.8% | 88.0% |
| Ovarian (337) | 85.5% | 86.9% |
| Breast Cancer (348) | 86.2% | 87.1% |
| Cervical Cancer (318) | 84.0% | 87.4% |
| Uterine Cancer (105) | 84.8% | 87.3% |
| Prostatic Cancer (31) | 80.6% | 87.5% |
| Brain Tumor (50) | 82.0% | 87.1% |
| Lymphoma (322) | 87.6% | 87.7% |
| Nasopharyngeal Cancer (121) | 81.0% | 87.1% |
| Other cancer (1593) | 85.6% | 86.8% |

Table 10 shows CDA values of non-small lung cancer samples at various stages and control sample, and corresponding sensitivity and specificity, which are higher than traditional methods, particularly at stage I.

TABLE 10
CDA technology demonstrates high sensitivity and
specificity for early stage screening of NSCLC

| | | | Average | Median | SD of | | |
|---|---|---|---|---|---|---|---|
| | | Sample | CDA | CDA | CDA | | |
| Group | | Size | (rel. units) | (rel. units) | (rel. units) | Sensitivity | Specificity |
| Control | | 248 | 33.98 | 34.72 | 5.50 | / | / |
| NSCLC | Stage I | 108 | 49.49 | 50.63 | 9.03 | 85.2% | 90.7% |
| | Stage II | 90 | 52.38 | 53.66 | 7.21 | 93.3% | 91.1% |
| | Stage III | 246 | 53.66 | 53.87 | 5.26 | 98.0% | 95.6% |
| | Stage IV | 388 | 52.45 | 52.96 | 6.11 | 95.1% | 95.2% |

Esophageal cancer is a cancer which still does not have a bio-marker and IVD screening method. In this investigation, CDA technology has been evaluated for esophageal cancer screening. Esophageal cancer results are summarized in Table 11. Results showed even at stage I, sensitivity and specificity are above 80%, far better than those by other technologies, which will have significant clinical meaning in catching esophageal cancer early.

TABLE 11
CDA technology demonstrates high sensitivity and specificity
for early stage screening of esophageal cancer

| | | | Average | Median | SD of | | |
|---|---|---|---|---|---|---|---|
| | | Sample | CDA | CDA | CDA | | |
| Group | | Size | (rel. units) | (rel. units) | (rel. units) | Sensitivity | Specificity |
| Control | | 248 | 33.98 | 34.72 | 5.50 | / | / |
| Esophageal | Stage I | 38 | 47.38 | 48.47 | 6.78 | 81.6% | 84.7% |
| Cancer | Stage II | 88 | 45.63 | 44.96 | 10.28 | 80.7% | 84.7% |
| | Stage III | 95 | 47.37 | 46.03 | 9.66 | 80.0% | 84.7% |
| | Stage IV | 63 | 54.37 | 53.22 | 16.04 | 85.7% | 85.1% |

### Follow-Up Data

CDA technology was utilized to screen ˜100,000 general populations. Based on CDA values, screened individuals were divided into three groups: high risk (beyond the standard, CDA value: ≥50.0), medium risk (need attention, CDA value: 42.0≤CDA<50.0), and low risk (in the normal range, CDA value: <42.0). Follow-up was conducted, and was able to make contact and obtain response from over 5809 individuals through the phone by professionally trained staff. For the period of follow-up (over one year to date), the high risk group was first contacted phone within 15 days after the report; the medium risk group was first contacted within 3 months after the report; and the low risk group was first contacted within 6 months after the report. For patients who are willing to accept follow-up visits, follow-up calls were or will be made every six months after the first phone visit.

Table 12A shows the initial follow-up data statistics. To date, follow-up was carried out on a group of individuals with low, medium, and high risk values, out of which 5809 individuals were able to have made contact and willing to share results from follow-up tests and diagnosis. Based on follow-up on 5809 individuals initially tested with low, medium and high CDA values and later confirmed by oncologists, Table 12B shows cancer cases screened out by CDA technology; Table 12C shows pre-cancer and benign tumor cases screened out by CDA technology; and Table 12D shows other disease cases screed out by CDA technology. As shown in Tables 12A-12D, at the time of the follow-up contact, 129 individuals were diagnosed by oncologists having cancer, 284 individuals were confirmed with pre-cancer and benign tumor, and 297 individuals were confirmed with other diseases. Follow-up is still on-going with remaining individuals.

TABLE 12A
Follow-up data statistics

| | No. of | No. with | | No. of | | Pre- | |
|---|---|---|---|---|---|---|---|
| Customer | CDA | contact | No. of | successfully | Cancer | cancer & | Other |
| Type | test | info | contacted | contacted | cases | benign tumor | disease |
| High Risk | 1361 | 854 | 862 | 460 | 63 | 61 | 41 |
| Medium Risk | 16651 | 12594 | 8980 | 4879 | 66 | 223 | 256 |
| Low Risk | 51712 | 39255 | 987 | 470 | — | — | — |
| Total | 69724 | 52703 | 10829 | 5809 | 129 | 284 | 297 |

710

TABLE 12B

| Cancer Cases | Number | Percent |
|---|---|---|
| Colorectal cancer | 25 | 19.4% |
| Lung cancer | 23 | 17.8% |
| Gastric cancer | 16 | 12.4% |
| Prostate Cancer | 16 | 12.4% |
| Breast cancer | 13 | 10.1% |
| Esophageal cancer | 4 | 3.1% |
| Renal carcinoma | 4 | 3.1% |
| Cancerous goiter | 4 | 3.1% |
| Liver cancer | 4 | 3.1% |
| Pancreatic cancer | 4 | 3.1% |
| Lymphoma | 4 | 3.1% |
| Cervical carcinoma | 3 | 2.3% |
| Cutaneum carcinoma | 3 | 2.3% |
| Bladder cancer | 1 | 0.8% |
| Throat cancer | 1 | 0.8% |
| Osteocarcinoma | 1 | 0.8% |
| Leukaemia | 1 | 0.8% |
| Cardiac cancer | 1 | 0.8% |
| Others | 1 | 0.8% |
| Total | 129 | |

TABLE 12C

| Pre-cancer & Benign tumor cases | Number | Percent |
|---|---|---|
| Pulmonary nodule | 56 | 19.7% |
| Thyroid nodule | 45 | 15.8% |
| Gastroduodenal disease | 43 | 15.1% |
| Hysteromyoma | 32 | 11.3% |
| Breast disease | 25 | 8.8% |
| Hepatic polyp | 22 | 7.7% |
| Colorectal polyp | 16 | 5.6% |
| Oophoritic cyst | 11 | 3.9% |
| Renal cyst | 7 | 2.5% |
| Gallbladder polyps | 5 | 1.8% |
| Otorhinolaryngologic tumor | 4 | 1.4% |
| Rostatic cyst | 4 | 1.4% |
| Hepatic hemangioma | 3 | 1.1% |
| Gastric polyps | 3 | 1.1% |
| Joint cyst | 2 | 0.7% |
| Meningioma | 1 | 0.4% |
| Gastric cyst | 1 | 0.4% |
| Bladder tumor | 1 | 0.4% |
| Liver nodule | 1 | 0.4% |
| Pulmonary calcification | 1 | 0.4% |
| Hepatic calcification | 1 | 0.4% |
| Total | 284 | |

TABLE 12D

| Other diseases | Number | Percent |
|---|---|---|
| Hypertension | 102 | 34.3% |
| Diabetes | 43 | 14.5% |
| Gastritis | 41 | 13.8% |
| Prostatitis | 18 | 6.1% |
| Coronary disease | 18 | 6.1% |
| Gynecological inflammation | 11 | 3.7% |
| Pneumonia | 11 | 3.7% |
| Bronchitis | 7 | 2.4% |
| Hyperlipidemia | 6 | 2.0% |
| Cholecystitis | 5 | 1.7% |
| Colorectal enteritis | 4 | 1.3% |
| Fatty liver | 4 | 1.3% |
| Heptatitis | 3 | 1.0% |
| Pulmonary tuberculosis | 3 | 1.0% |
| COPD | 2 | 0.7% |
| Lung fiber | 2 | 0.7% |
| Gallstone | 2 | 0.7% |
| Hyperthyyreosis | 2 | 0.7% |
| Nephrtis | 2 | 0.7% |
| Lithangiuria | 2 | 0.7% |
| Uremia | 1 | 0.3% |
| Thyromegaly | 1 | 0.3% |
| Rectal fistula | 1 | 0.3% |
| Sjogren's disease | 1 | 0.3% |
| Pleural thickening | 1 | 0.3% |
| Pulmonary embolism | 1 | 0.3% |
| Dematitis | 1 | 0.3% |
| Lympy node hyperplasia | 1 | 0.3% |
| Pulmonary shadow | 1 | 0.3% |
| Total | 287 | |

In addition, in initial retrospective studies, CDA test results on Caucasian group showed comparable sensitivity and specificity as those on Chinese Han ethnic group.

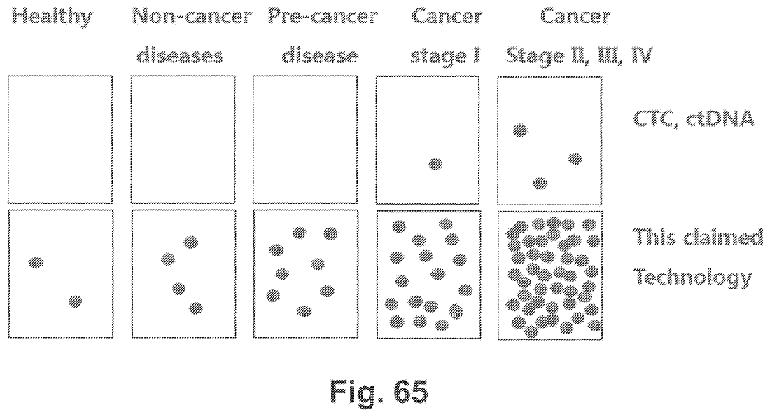

FIG. 65 shows a schematic comparing CDA technology with other cancer detection technologies, in which number of dots are proportional to detection signal. Unlike traditional cancer detection technologies which have relatively low signal to noise ratio, and some of them have signals starting when solid tumor has been formed. In contrast, signal at a CDA technology starts with health group and increases statistically significantly with disease progression, indicating that CDA technology is potentially a viable technology for pre-cancer and early stage cancer detection.

While the functions and properties of bio-physics have played a critical role in physiology, they have not been extensively utilized in the field of IVD of cancer, which has traditionally been more heavily replied upon bio-chemistry, immunology, and genomics. Prior work has not elucidated how electrical properties evolve from a normal state to cancerous state, nor it has been developed into a practical cancer detection tool. This work represents a novel approach and breakthrough in the field cancer detection. Results demonstrated that this technology has unique advantage to detect cancer early, and can be an effective approach to track disease progression, as it showed statistical difference between healthy group and disease group, and between disease group and cancer group. Compared with traditional approaches, the current approach detects a signal which is much more foundational and it is in existence in all human being including healthy individuals. Therefore, its signal is much earlier in nature in detecting occurrence of cancer. Further, micro- electrical current has shown to decrease significantly from healthy group to disease group and from disease to cancer group, making it ideal for early stage cancer detection and tracking diseases leading to cancer.

Results from tests (a) using samples with increasing amount of cancer cells, (b) using samples with increasing amount of bio-marker concentration CEA, and (c) with samples with and without an assay which is known to cause a molecular level reaction showed that CDA values are proportional to increasing amount of cancer cells and bio-marker CEA concentrations. In addition, CDA values are dependent on with and without molecular level reactions. Based on the above observations, it can be stated that CDA values are a function of cellular, protein, and molecular levels (as shown in FIG. 50).

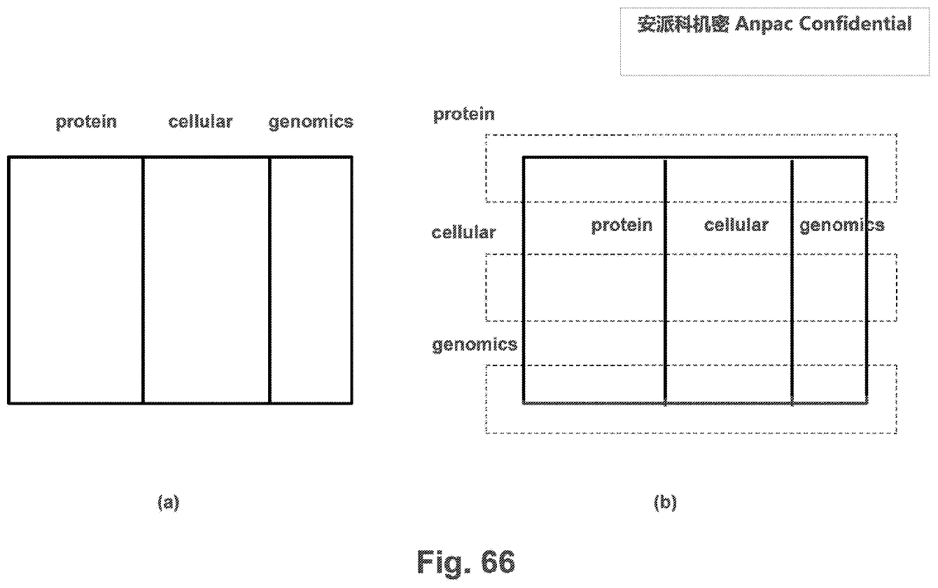

FIG. 66 shows that CDA technology is a multi-level and multi-parameter test that can also be carried out in conjunction with other tests including bio-markers (protein level), CTC (cellular level), and/or ct-DNA and other DNA based tests (genetic tests). While CDA is a function of multiple levels as stated above, it is also an advantage sometimes to perform CDA tests in conjunction with other cancer tests to obtain additional combined test results such as combined tests with bio-markers, CTCs, and genomics tests as shown in FIG. 66, where additional dimensional information can be obtained.

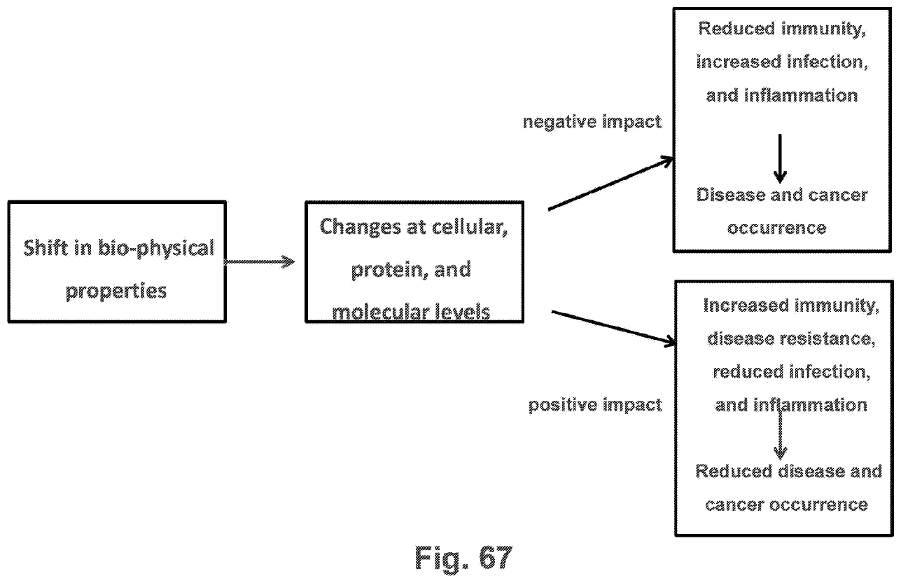

FIG. 67 shows a schematic of a proposed model, in which shift in bio-physical properties such as electrical properties cause changes at cellular, protein, and molecular (gene) levels which result in changes at immunity and inflammation, and likelihood (or less likelihood) of diseases and cancer occurrence.

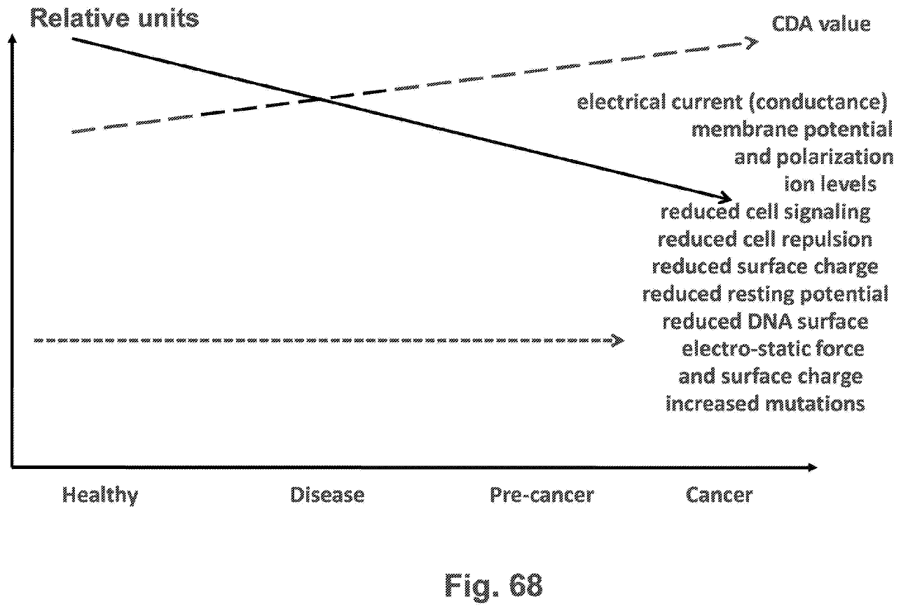

FIG. 68 shows that as CDA increases and electrical current, conductance, ion level, membrane potential and polarization decrease, a number of cellular level (cell signaling, cell repulsion, resting potential and cell surface charge decrease) and molecular level (DNA surface charge decrease, quantum mechanical effect change, and DNA mutation increases) properties degrade, resulting in increased disease and cancer occurrence.

Initial data indicates that this novel technology is correlated to protein level (bio-chemistry), cellular level and molecular level expressions, and it has multi-level, and multi-parameter characteristics. Having demonstrated viability of this new technology for pre-cancer and early stage cancer detection, possible mechanism can be further proposed. A scheme of cells, proteins, and genetic components (DNA, RNA, etc.) and their surrounding liquid media (e.g., blood) is described above and provided in FIG. 60. First of all, as one of the important bio-physical parameters, electrical properties (which include but not limited to electrical current, conductance, quantum mechanical effects, electrical field, resting potential of cells, capacitance, cell surface charge, and electro-static force) affect at cellular, protein, and molecular levels. Specifically, electrical properties including micro-electrical current, conductance, and quantum mechanical effects not only impact cell surface properties, they also affect how cells interact each other (for example, repulsion and attraction between cells) as well as possibly cell signaling and shifting resting potential of cells. Also, electrical properties modify protein surface phase and structure. In addition, shift in micro-electrical current (and accordingly conductance) confirmed in the work in blood and/or change of quantum mechanical effects may possibly affect functioning and replications of DNA (increased mistakes in gene replications), and even causing increased frequency of DNA mutations. This conjuncture is directly and indirectly support by: (a) recent bio-physics work in mechanical stress studies indicated correlations between mechanical aspects in cellular structure and nuclear and chromatin organization including altered genomic program, and earlier studies appear to show that resting potential could be a potential important factor of the microenvironment regulating the balance between normal growth and carcinogenesis, (b) a shift in electrical property likely impacts surface charge of and electro-static force exerting on three dimensional DNA double helix structures and, (c) bio-physics work in this study in the area of electrical properties also indicated correlation between electrical property shift and occurrence of cancer which is often a result of increased gene mutation; (d) quantum mechanical effects affect gene replications and mutations. Based on experimental data presented in this work and above direct and indirect evidences, a hypothesis on cancer occurrence is proposed as follows. As micro- electrical current is reduced, at cellular level, cell surface charge as well as repulsive force between cells is reduced, cell signaling also is reduced and likely becomes less efficient and effective, and resting potential is shifted. All of the above stated developments at cellular level are not desirable. At molecular level, with reduction in micro-electrical current and/or change of quantum mechanical effects, mutation frequency may increase due to likely reduced electro-static force and surface charge on double helix three dimensional structures and amino acids surfaces, and possibly impacting quantum mechanical effects at DNA microscopic level, resulting in increased replication errors. The above hypothesis on the negative effects at multi-biological levels caused by reduced micro- electrical current (and conductance) of blood match our experimental observations and data in retrospective investigations on healthy group, disease group, and cancer group samples, and also agree with results from initial follow-up studies on general population screening. Since this model is based upon electrical properties of blood, it is named electrical model of cancer (EMOC).

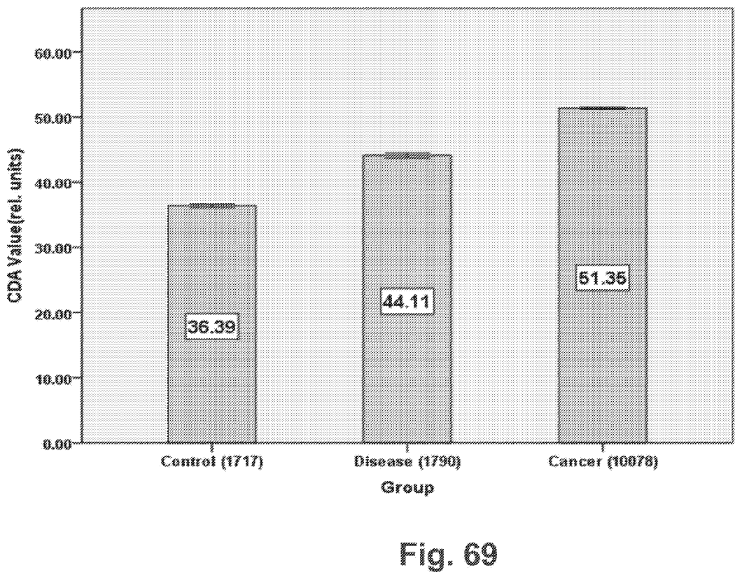

Compared with other traditional cancer detection technologies, CDA technology has many unique features and clear advantages. First, many existing technologies detect cancer signals after cancer has already formed which make those technologies ineffective for early stage cancer detection, while CDA technology detects a bio-physical parameter which exists in healthy individuals and rises as the risk of cancer increases (as shown in FIG. 69), where CDA values for healthy group, disease group and cancer group showed statistical difference (P<0.001). Such rise in CDA values is statistically significant before and during early stage of cancer, making CDA technology far more suited for early stage cancer detection. Secondly, unlike most of existing cancer detection technologies which are based on detecting a single level (for example, bio-marker at protein level and CTC at cellular level) and even a single parameter, CDA technology is a multi-level and multi-parameter technology which is much more comprehensive and contains much more information, making it more accurate. Thirdly, CDA technology detects micro- electrical current signal which is more fundamental with a high signal to noise ratio, and decrease in micro- electrical current likely to be the cause for loss of immunity and increasing occurrence of cancer which can be detected well before cancer is formed, in contrast to most of the exiting detection technologies which pick up signal when cancer has already occurred and in many cases are already at late stage cancer.

In addition, based on CDA value dependent disease progression behavior (disease progresses with decreasing micro- electrical current of the blood sample); based on the above proposed hypothesis, new model for cancer occurrence is proposed as follows. In this new model, as a major bio-physical parameter, the shift in electrical properties of blood, specifically, decreasing in micro- electrical current and/or changing quantum mechanical effects (which affect gene replications and mutations) is causing negative effects at multi-levels which include (1) reduced surface charge, cell repulsion, and cell signaling efficiency at cellular level, and (2) reduced electrostatic force, DNA surface charge, and possibly increased mutation at DNA level. Further, it is hypothesized that reduced micro-electrical current (and conductance) also causes reduced surveillance capability of T cells for cancer cell detection and reduced immunity which increase occurrence of cancer. The above hypothesis is supported by data collected in this work showing that decreasing (increasing CDA values) in micro- electrical current is correlated with disease progress from healthy group to disease group, from disease group to pre-cancer group, and from pre-cancer group to cancer group.

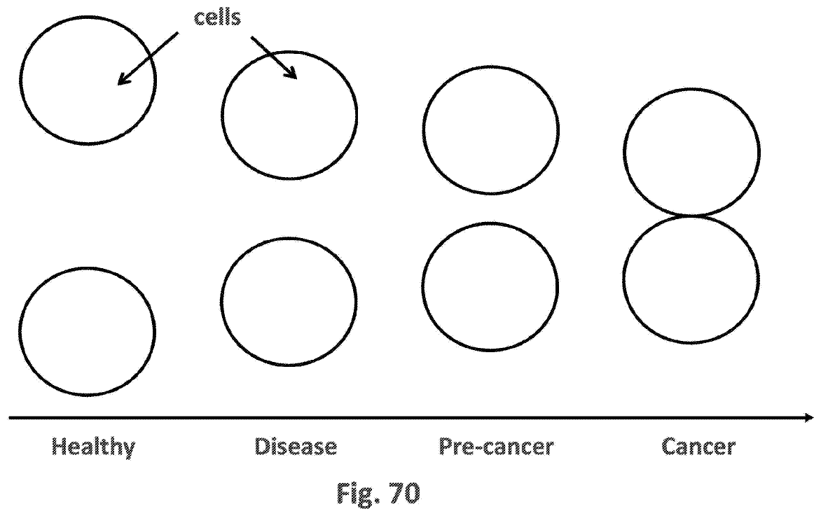

FIG. 70 shows that as electrical current and conductance decrease (ion (e.g., potassium, chloride, sodium, and calcium) concentration or net ion concentration or charge decreases), a number of cellular level (cell signaling, cell repulsion, resting potential, membrane potential and cell surface charge decrease) properties change and degrade. For example, cell surface charge decreases, resulting in reduction in repulsive force between cells and decreased distance between cells. Finally, in cancer stage, cells lose concept of space and boundary, and collapse to each other (sticking/stacking to each other), in which repulsive force between cells are reduced due to reduced cell surface charge. Therefore, repulsive force between cells due to surface charge on cell surfaces are very important.

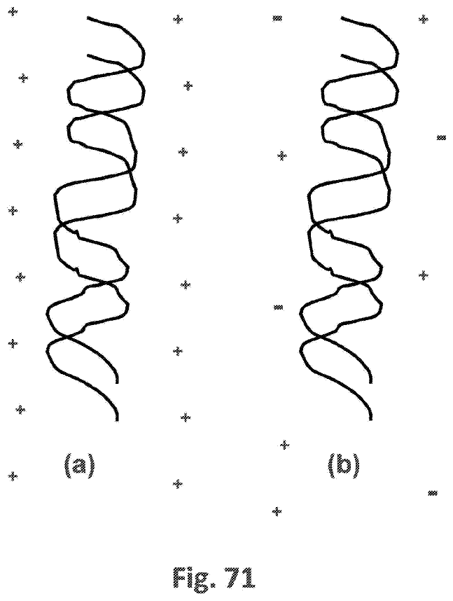

In this invention, changes in electrical properties in blood and DNA level can be used as a tool for disease detection. As electrical current and conductance decrease, a number of molecular level (DNA surface charge decreases, quantum mechanical effect change, and DNA mutation increases) properties degrade, resulting in increased disease and cancer occurrence. As shown in FIG. 71, in a sample from a health case (a), both surrounding and DNA surface have higher charge, while in a sample from a cancer case (b), both surrounding and DNA surface have less charge, possibly overall negative charge. Since for DNA double helix structures, DNA surface charge and electrical properties of the media may affect its electro-static force and hence 3-dimensional structures, as well as quantum mechanical effects (at atomic level, and with spacing between adjacent amino acids is only at a few angstrom), change at electrical properties of DNA surrounding media and/or DNA surface charge may affect DNA replications and cause increased replication error rate and gene mutations.

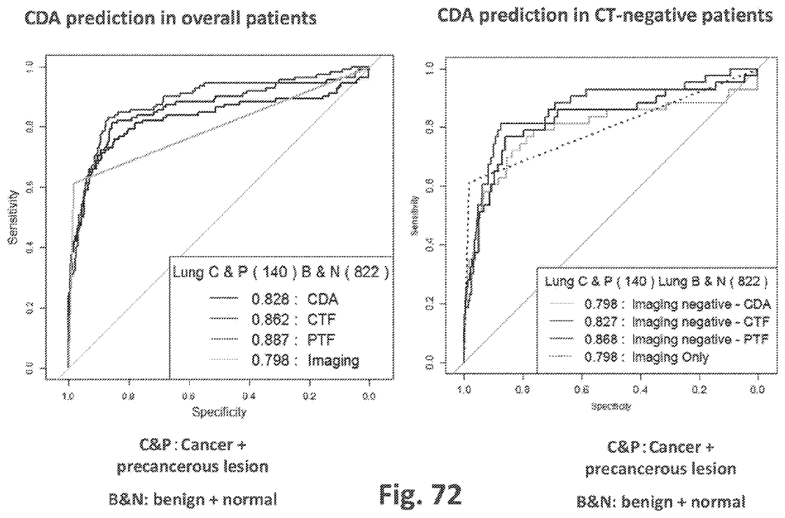

Furthermore, the new technology according to this invention can also be used in assisting in diagnosis, such as assisting in diagnosis of lung cancer. As shown in FIG. 72, compared with CT, this novel technology (parameters of CDA, CTF and PTF) has better and higher sensitivity and specificity. Additionally, its ROC is better than that of CT imaging.

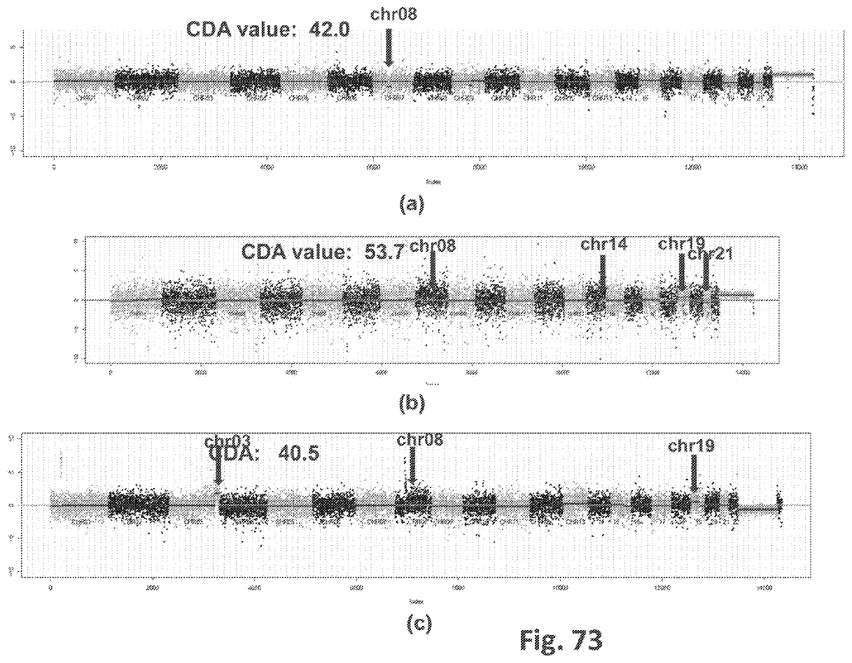

As also shown in FIG. 73 that the CDA values appear to correlate with mutation frequency for (a) healthy, (b) lung cancer just after diagnosis and before surgery, and (c) after surgery and treatment individuals/groups.

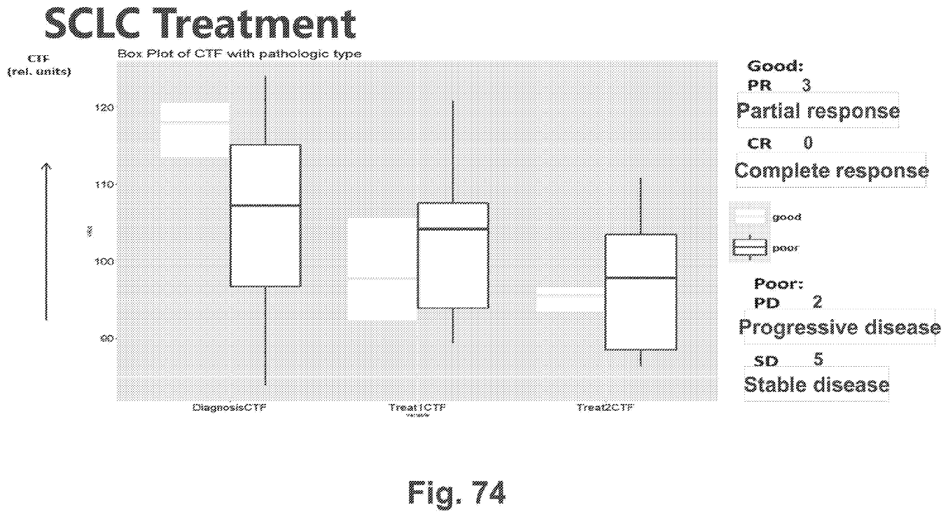

Initial clinical study results show that the novel technology according to this invention is capable of evaluating effectiveness of drug treatment of cancer. In this case (e.g., as shown in FIG. 74), this novel cancer detection technology is used for prognosis of a targeted drug treatment of small cell lung cancer at three stages—i.e., after diagnosis, after phase 1 treatment, and after phase 2 treatment. In FIG. 74, CTF is a parameter of this novel technology.

One of key aspects of this invention is that the bio-physical properties and its associated behaviors disclosed in this novel work are of common to a large number of cancer types, and can be used for detection of a large number of cancer types, making the disclosed method a viable technology for cancer screening, assisting in diagnosis, prognosis, therapy selection and reoccurrence detection.

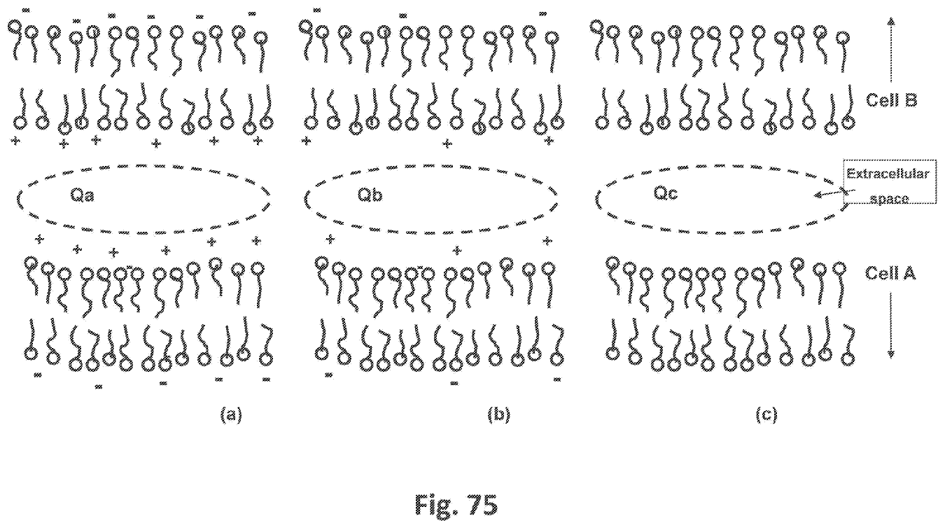

FIG. 75 is a schematic of cell membranes with intracellular and extracellular regions, with decreasing membrane potential, net charge Q in extracellular region (and membrane polarization) from (a) to (b) to (c), and net charge Qa<Qb<Qc. Based on experimental data in this work in electrical conductivity in whole blood and serum, which showed decreasing electrical conductivity (decreasing electrical current and electrical charge) mainly due to properties in extracellular regions from healthy group to disease group to cancer group, it is claimed that schematic (a) corresponding to health condition, schematic (b) corresponding to disease condition, and schematic (c) corresponding to cancer condition.

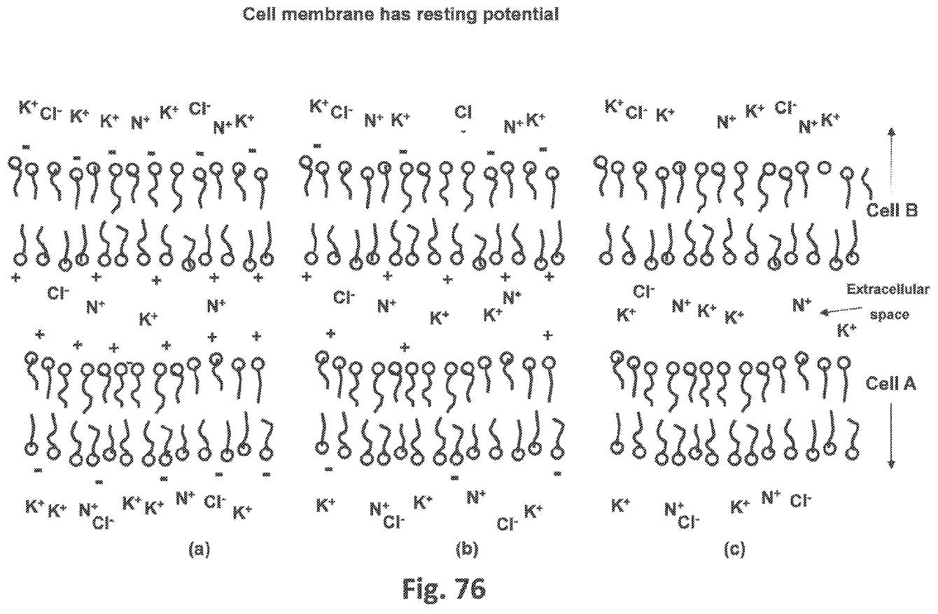

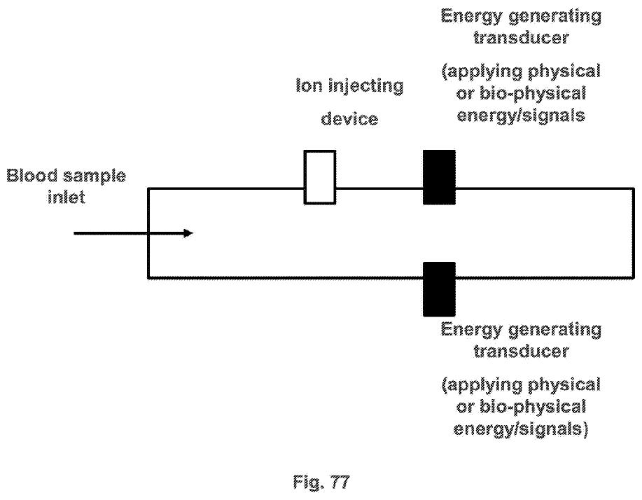

FIG. 76 shows a schematic of membranes of two cells showing membrane potential, intracellular space, and extracellular space. As shown in FIG. 76, schematics (a), (b) and (c) represent healthy, disease and cancer cases including membrane potentials, ion distributions, and net charges, with decreasing blood conductivity (measured values), membrane potential and polarization, and net charge in extracellular region. Notably, the medical device according to the present invention can treat a biological subject (e.g., a blood sample) by reversing its situations presented in FIGS. 76 and 77, e.g., from situation (c) to (b) and to (a) as shown in FIG. 76 or 77.

As shown in FIG. 76, high permeability of potassium ions into cells (and high concentrations of sodium and chloride ions in extracellular region) create differences in concentrations of ions on opposite sides of a cellular membrane and hence an electrical potential across membrane layer. In the local region or near field, it is not electrically neutral, while in a larger scale, it is electrically neutral. By probing electrical properties at a local region or near field, information relating to cell properties including but not limited to electrical conductivity, electrical resistance, ion concentrations, ion levels, ion permeability, membrane ion channel properties, resting membrane potential, trans-membrane potential, depolarized trans-membrane potential, membrane potential, cell surface charge, electro-static force, electrical field, electro-magnetic field, and quantum mechanical effects can be obtained directly or indirectly.

In one embodiment, utilizing a micro-fluidic device with micro-channels and sensitive sensors, electrical properties of blood samples at near field of cells illustrated in above figure (schematic of cellular membranes) can be measured, and related electrical properties including electrical current across the region, trans-membrane potential, and ion levels (potassium ions, sodium ions, chloride ions, calcium ions, and nitride ions) can be directly and indirectly measured. Since disease state of mammals is related to the above-mentioned cellular bio-physical properties (and DNA, RNA and other biological entities in the cells), the above inventive measurement technology can be used to detect diseases including pre-cancer and cancer diseases. The membrane potential can regulate the balance between normal cellular activities including normal growth and replications, and carcinogenesis. As such, both ion level and concentration (potassium ions, sodium ions, chloride ions and calcium ions) and membrane potential could be used as a new, novel bio-marker for cancer prevention and early stage cancer detection.

The present invention provides a new cancer detection technology using a bio-physical approach based on electrical properties of liquid samples for IVD applications. In this new technology, a micro- electrical current is detected which has shown to be very effective in detecting pre-cancer and early stage cancer. This technology has the advantages of screening/detecting or even treating multiple types of diseases (e.g., multiple cancer types) at the same time, detecting cancer early, high sensitivity and specificity, covering a wide range of cancer types, and relatively simple and cost effective. Based on how CDA values are correlated to control, disease and cancer groups in this work, and possible effects of electrical properties in blood on disease progression, a new hypothesis on cancer occurrence model is proposed in which a reduction in blood micro electrical current (and conductance) and/or a change of quantum mechanical effects is proposed to cause a number of negative effects at cellular and molecular levels, resulting in reduced cell to cell signaling, cell to cell repulsion, and immunity, and increased gene mutation frequency, and hence increased occurrence of cancer.

While for the purposes of demonstration and illustration, the above cited novel, detailed examples show how microelectronics and/or nano-fabrication techniques and associated process flows can be utilized to fabricate highly sensitive, multi-functional, powerful, and miniaturized detection devices, the principle and general approaches of employing microelectronics and nano-fabrication technologies in the design and fabrication of high performance detection devices have been contemplated and taught, which can and should be expanded to various combination of fabrication processes including but not limited to thin film deposition, patterning (lithography and etch), planarization (including chemical mechanical polishing), ion implantation, diffusion, cleaning, various materials, combination of processes and steps, and various process sequences and flows. For example, in alternative detection device design and fabrication process flows, the number of materials involved can be fewer than or exceed four materials (which have been utilized in the above example), and the number of process steps can be fewer or more than those demonstrated process sequences, depending on specific needs and performance targets. For example, in some disease detection applications, a fifth material such as a biomaterial-based thin film can be used to coat a metal detection tip to enhance contact between the detection tip and a biological subject being measured, thereby improving measurement sensitivity.

Applications for the detection apparatus and methods of this invention include detection of diseases (e.g., in their early stage), particularly for serious diseases like cancer. Since cancer cell and normal cell differ in a number of ways including differences in possible microscopic properties such as electrical potential, surface charge, density, adhesion, and pH, novel micro-devices disclosed herein are capable of detecting these differences and therefore applicable for enhanced capability to detect diseases (e.g., for cancer), particularly in their early stage. In addition to micro-devices for measuring electrical potential and electrical charge parameters, micro-devices capable of carrying out mechanical property measurements (e.g., density) can also be fabricated and used as disclosed herein. In mechanical property measurement for early stage disease detection, the focus will be on the mechanical properties that likely differentiate disease or cancerous cells from normal cell. As an example, one can differentiate cancerous cells from normal cells by using a detection apparatus of this invention that is integrated with micro-devices capable of carrying out micro-indentation measurements.

Another aspect of this invention provides treatment of diseases. FIG. 77 is a schematic of an apparatus for disease treatment using physical or bio-physical approaches. This treatment can be used in vitro. The treatment can be carried out on a sample of body fluid, such as blood. The treatment can utilize a constant or varying (or alternating) energy and/or field. The treatment can be carried out using circulating blood from a patient body. As shown in FIG. 77, the apparatus for treating a disease comprises a channel in which the biological subject (e.g., blood sample) passes through; an ion injecting device for injecting a desired amount of additives (e.g., ions); and one or more generators (transducers) for applying at least one type of energy and/or field (e.g., physical or bio-physical energy/signal or field) onto the biological subject. The apparatus may also include one or more detectors for detecting a physical or bio-physical signal of the biological subject.

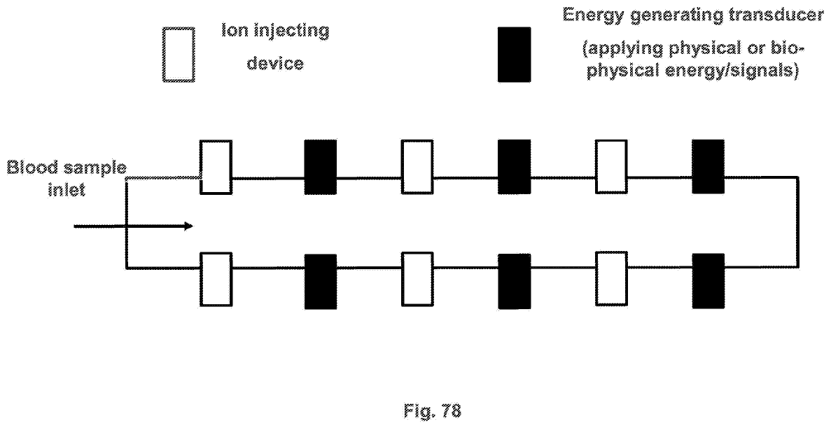

FIG. 78 shows another exemplary apparatus for disease treatment using physical or bio-physical approaches. As shown in FIG. 78, the apparatus comprises a channel in which the biological subject (e.g., blood sample) passes through; multiple ion injecting devices each for injecting a desired amount of additives (e.g., ions); and multiple generators (transducers) for applying at least one type of energy and/or field (e.g., physical or bio-physical energy/signal or field) onto the biological subject. This treatment also can be used in vitro. The treatment can be carried out on a body fluid (e.g. blood). In some embodiments, the treatment can be carried out using circulating blood from the body. The treatment can utilize a constant or varying (or alternating) energy and/or field. The apparatus is configured to apply physical or bio-physical energy and/or field to body fluid while injecting the proper amount of additive(s) such as ions into the body fluid.

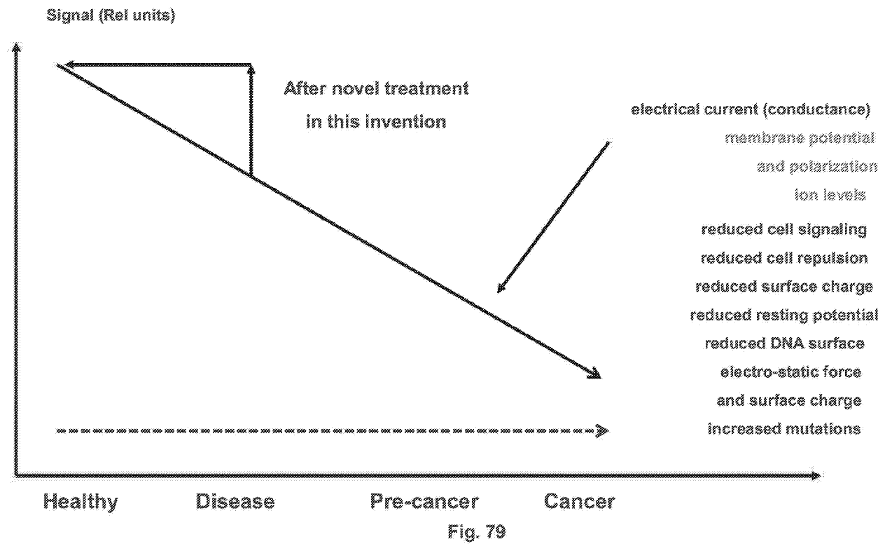

FIG. 79 illustrates the change of the property in a biological subject after the disease treatment of this invention. As shown in FIG. 79, as electrical current, conductance, ion level, membrane potential and polarization decrease, a number of cellular level (cell signaling, cell repulsion, resting potential and cell surface charge decrease) and molecular level (DNA surface charge decrease, quantum mechanical effect change, and DNA mutation increases) properties degrade, resulting in increased disease and cancer occurrence. In the opposite way, the treatment of this invention (e.g., by applying physical or bio-physical energy and/or filed while injecting the proper amount of additive(s) such as ions) may impact at least one of the following properties in the biological subject: electrolyte concentration and distribution, potassium ion concentration and distribution, sodium ion concentration and distribution, chloride ion concentration and distribution, calcium ion concentration and distribution, net charge in extracellular region, ion concentration in extracellular, gravitational field, mechanical field, quantum mechanical field, magnetic field, electro-magnetic field, electrical field, electrical current, electrical resistance, electrical conductance, capacitance, membrane ion channel properties, resting membrane potential, trans-membrane potential, depolarized trans-membrane potential, membrane voltage, and membrane potential - thereby resulting in deceased level of disease and cancer state. After the treatment, the properties of the patient's biological subject may return to a healthier state. For instance, the properties of the biological subject return from a cancer state to an earlier cancer stage, a pre-cancer state, a non-cancer disease state or a healthy state; the properties of the biological subject return from a pre-cancer state to a non-cancer disease state or healthy state; or the properties of the biological subject return from a non-cancer disease state to a healthy state. The novel cancer treatment of this invention can use a low dosage and/or a weak field and/or energy for treatment, thereby an much improved treatment approach with lower costs, far less side efforts, easier recovery, capability of cancer prevention, improved survival rate and ease of use.

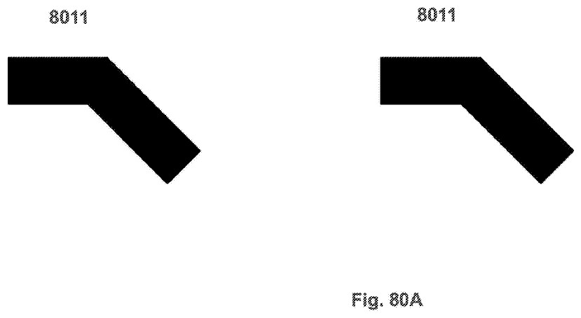

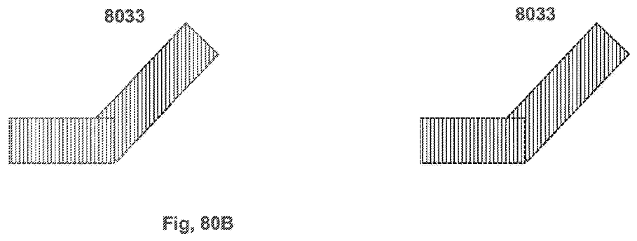

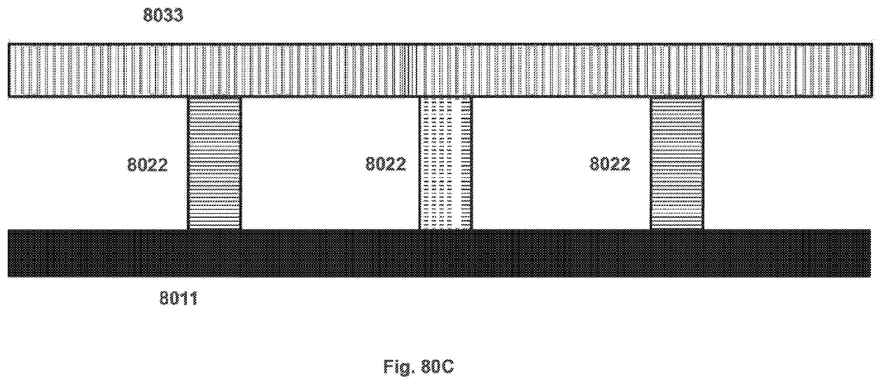

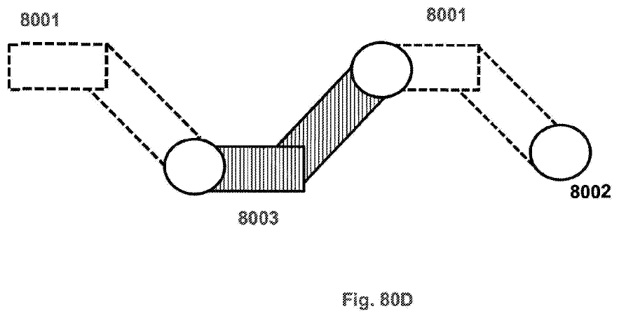

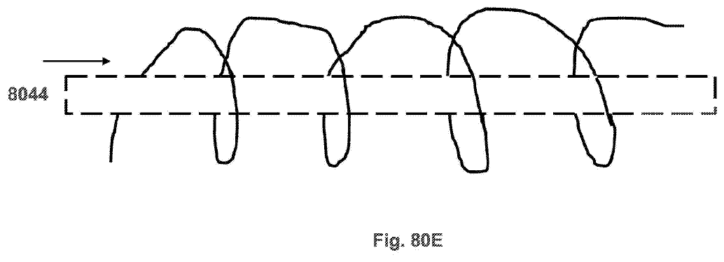

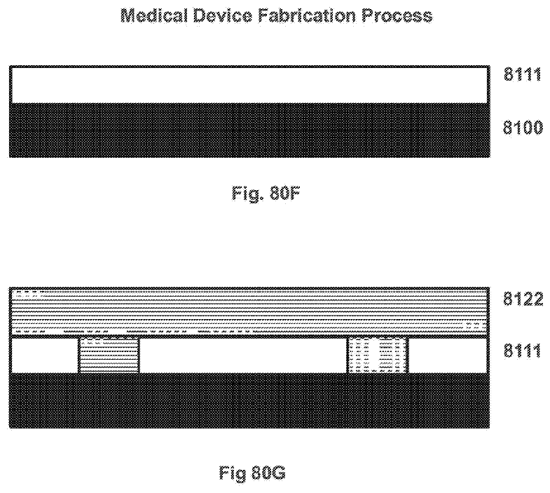

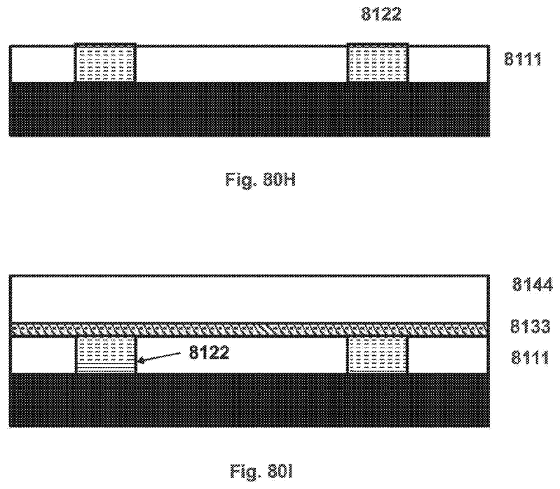

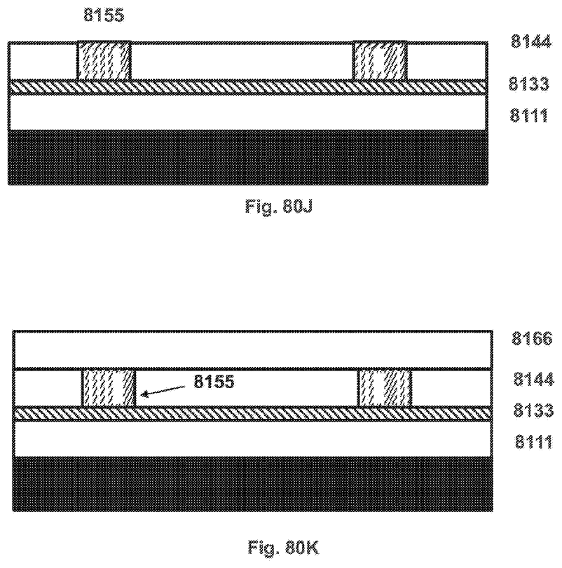

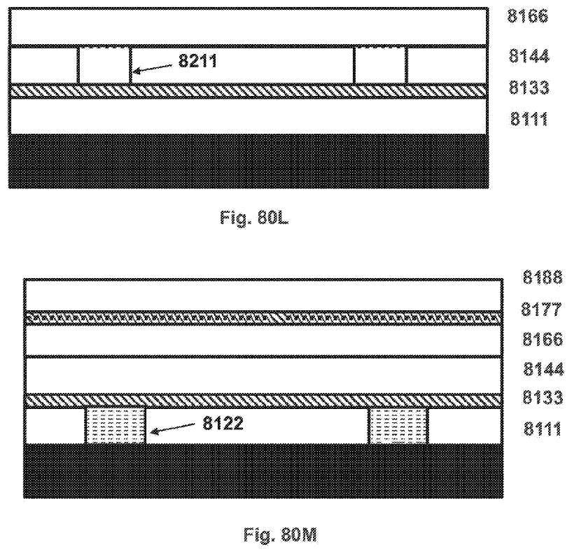

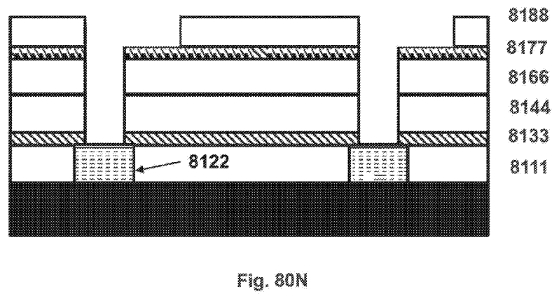

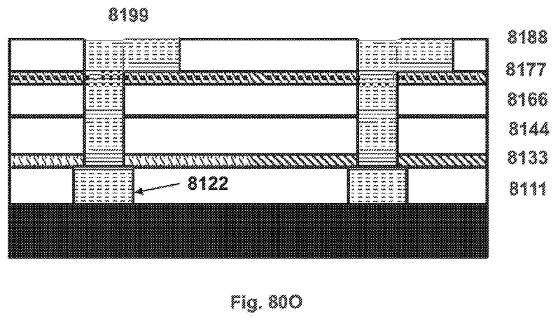

FIGS. 80A-80O illustrate an apparatus for treating a disease, with a coil surrounding channel which is capable of applying an energy onto the biological subject. FIG. 80A illustrates the bottom layer of a conductive coil 8011, which is surrounded by insulating material. FIG. 80B illustrates the top layer of a conductive coil 8033, which is surrounded by insulating material. FIG. 80C illustrates the middle layer including conductive plugs (8022) surrounded by insulating material. Conductive plugs are part of conductive coils connecting bottom conductive layer (8011) and top conductive layer (8033). FIG. 80D is a top view of the conductive coil fabricated by the novel semiconductor or integrated circuit manufacturing methods. Features in solid line are top conductive layer (8033) while features in dotted line are bottom conductive layer (8011). Solid circles are conductive plugs (8022) connecting top and bottom conductive layers. FIG. 80E shows a three-dimensional view of a conductive coil fabricated by the novel semiconductor or integrated circuit methods, which is wrapping around a channel (8044) along which a body fluid (e.g., blood) flow through or stay for a desired period of time, for treating a disease. The conductive coils are connected through conductive features 8011, 8022, and 8033 as described above.

FIGS. 80E-80O illustrate novel fabrication processes for fabricating an apparatus for treating a disease, with a coil surrounding channel. As shown in FIG. 80F, an insulating layer 8111 is first deposited on a semiconductor or insulating substrate 8100. This cross-section shows a future coil area. Next, as shown in FIG. 80G, trenches are etched into insulating layer 8111, and conductive material(s) 8122 is deposited. The following step is to etch back or polish back conductive material(s) 8122 (shown in FIG. 80H). Afterwards, a thin stop layer (such as silicon nitride or poly-silicon) 8133 is deposited, and subsequently layer 8144 which is the same type of insulating material(s) as 8111 is deposited (as shown in FIG. 801). The next step is shown in FIG. 80J, to etch layer 8144 (applying lithography and etch) and form trench, deposit a sacrificial layer 8155, and then etch back or polishing back layer 8155. Notably, the cross-sectional view of the channel in FIG. 80J is at a different location on the chip from the above cross-sectional view (e.g., in FIG. 801). Then, as shown in FIG. 80K, an insulating layer 8166 is deposited. The material of layer 8166 is the same type of insulating material(s) as 8111.

As shown in FIG. 80L, an etching process is used to etch a small hole through layer 8166, and then an etching process is used to etch off sacrificial material 8155, thereby forming micro-channels 8211 in layer 8144. Next, as shown in FIG. 80M, a thin etch stop layer 8177 (which has the same material as layer 8133) and an insulating layer 8188 (which is the same type of insulating material(s) as 8111) are subsequently deposited. As shown in FIG. 80N, layer 8188 is patterned and etched to form trench, and then patterned and further etched to form hole through layers 8177, 8166, 8144, 8133 (using different etch chemistries with one chemistry for layers 8166, 8144, and another chemistry for layers 8133 and 8177) which stops on layer 8122. Alternatively, the second processing option is to use double lithography exposure (for trench area and deep hole area respectively) and do a double-etch, thereby etching trench area first and then hole area.

Finally, as shown in FIG. 80O, deposit conductive layer 8199 (which can be the same as conductive layer 8122) is deposited, thereby making contact with bottom conductive layer 8122. As such, conductive layers 8122 (bottom layer) and 8199 (contact and top layer) form a coil surrounding micro-channels 8211. The top view and three-dimensional schematic view of the coil surrounding micro-channel are described above in FIGS. 80D and 80E.

Although specific embodiments of this invention have been illustrated herein, it will be appreciated by those skilled in the art that any modifications and variations can be made without departing from the spirit of the invention. The examples and illustrations above are not intended to limit the scope of this invention. Any combination of detection apparatus, micro-devices, fabrication processes, and applications of this invention, along with any obvious their extension or analogs, are within the scope of this invention. Further, it is intended that this invention encompass any arrangement, which is calculated to achieve that same purpose, and all such variations and modifications as fall within the scope of the appended claims.

All publications or patent applications referred to above are incorporated herein by reference in their entireties. All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

1. An apparatus for simultaneously detecting presence or monitoring progression of two or more different types of cancer in a biological subject, comprising:
a channel or chamber in which the biological subject passes through;
at least one detector placed partially or completely alongside one or more side walls of the channel or chamber, wherein the at least one detector is configured to detect at least one bio-physical or physical property of the biological subject, and the detected bio-physical or physical property is collected for analysis to determine whether each of the two or more types of cancer is likely to be present with the biological subject or to determine the status of each cancer, thereby providing the ability to simultaneously determine or monitor progression of the two or more types of cancer,
wherein the bio-physical or physical property exits in each of the two or more different types of cancer, and is able to differentiate a normal or non-cancerous sample from abnormal samples with the cancers.

2. (canceled)

3. (canceled)

4. (canceled)

5. (canceled)

6. The apparatus of claim 1, wherein the apparatus detects one or more bio-physical or physical properties each selected from the group consisting of electrolyte, potassium ion concentration, sodium ion concentration, chloride ion concentration, calcium ion concentration and distribution, net charge in extracellular region, ion concentration in extracellular, gravitational field, mechanical field, quantum mechanical field, magnetic field, electro-magnetic field, electrical field, electrical current, electrical resistance, electrical conductance, capacitance, membrane ion channel property, resting membrane potential, trans-membrane potential, depolarized trans-membrane potential, membrane potential, magnetic field, electrical field, electro-magnetic field, and quantum field in the biological subject.

7. The apparatus of claim 6, wherein the apparatus directly or indirectly measures one or more bio-physical or physical properties comprising the quantum mechanical effects that affect gene replications and mutations, ions, ion levels, ion concentration, or ion permeability.

8. (canceled)

9. (canceled)

10. (canceled)

11. (canceled)

12. The apparatus of claim 7, wherein the ion comprises potassium ion, sodium ion, chloride ion, nitride ion or calcium ion; or wherein the ions are measured by a bio-chemistry or electrode method.

13. The apparatus of claim 12, wherein the ion comprises potassium ion.

14. (canceled)

15. The apparatus of claim 1, wherein the biophysical or physical property is related to and responsible for cell to cell interactions, cell signal, cell surface properties, cell electro-static force, cell repulsive force, DNA surface properties, DNA surface charge, DNA surrounding media electrical properties, quantum mechanical effects, gene mutation frequencies, or quantum mechanical effects.

16. The apparatus of claim 1, wherein the biological subject is a liquid sample comprising a body fluid or tissue; wherein the body fluid comprises whole blood, serum, plasma, sweat, tears, saliva, or urine.

17. (canceled)

18. (canceled)

19. (canceled)

20. (canceled)

21. (canceled)

22. The apparatus of claim 1, wherein the determination is by comparing the biophysical information of the detected biological subject with the same biological information of a confirmed disease-free or diseased biological subject; the status of each of the cancers comprises healthy stage, non-cancer disease stage, pre-cancer stage, early stage cancer stage, and mid to late stage cancer stage, with statistically significant detection or monitoring between any of the two stages; or the apparatus simultaneously detects presence or monitoring progression of three or more types of cancer in the biological subject.

23. (canceled)

24. (canceled)

25. The apparatus of claim 1, wherein the cancers comprise lung cancer, liver cancer, colorectal cancer, breast cancer, gastric cancer, esophageal cancer, brain tumor, prostate cancer, blood cancer, intestine cancer, stomach cancer, cervical cancer, ovarian cancer, rectum cancer, colon cancer, nasopharyngeal cancer, cardiac carcinoma, uterine cancer, oophoroma, pancreatic cancer, or circulating tumor cells.

26. The apparatus of claim 1, further comprising an additional device for adjusting the physical or bio-physical property in the biological subject.

27. The apparatus of claim 26, wherein the physical or bio-physical property is first measured and then adjusted.

28. (canceled)

29. (canceled)

30. The apparatus of claim 26, wherein the additional device adjusts the current to a higher value, adjusts the electrical conductance to a higher value, adjusts the electrical resistance to a lower value, or alters the quantum mechanical effect.

31. The apparatus of claim 30, wherein a reagent is injected to adjust bio-physical properties of the blood.

32. The apparatus of claim 31, wherein the reagent comprises ions, oxidizers, and components to impacting one or more electrical properties of the blood; the electrical property comprises electrical current, electrical conductance, capacitance, electrical resistance, ion types and/or concentrations, or quantum mechanical effect.

33. (canceled)

34. The apparatus of claim 31, wherein the reagent is a drug capable of adjusting the biological properties of the blood; or capable of releasing, upon intake, ions and charged components and capable of adjusting electrical properties of the blood; wherein the electrical property comprises electrical current, electrical conductance, capacitance, electrical resistance, ion types and/or concentrations, or quantum mechanical effect.

35. (canceled)

36. (canceled)

37. The apparatus of claim 1, wherein at least one bio-marker is added to the liquid sample for property measurement; wherein the bio-marker provides at least some indicative information of risks of two or more different types of cancer occurrence at a given organ and location; or the detected property is analyzed in conjunction with information and data obtained from test(s) comprising of bio-marker tests, genomics tests, circulating tumor DNA, circulating free tumor DNA, and circulating tumor cell tests, and overall cancer risks and location(s) of possible cancer occurrence are obtained.

38. (canceled)

39. (canceled)

40. The apparatus of claim 26, wherein the apparatus comprises an ion injector, configured to add a desired amount of ions to the biological subject.

41. The apparatus of claim 40, wherein the ions comprise potassium ion, sodium ion, chloride ion, nitride ion, or calcium ion.

42. (canceled)

43. The apparatus of claim 1, wherein the apparatus comprises one or more channels, and the one or more channels comprise one or more detectors and are configured to detect one or more bio-physical or physical properties of the biological subject.

44. The apparatus of claim 43, wherein the apparatus further comprises one or more ion injectors on sidewalls, and each of the one or more ion injectors is configured to add a desired amount of ions to the biological subject; wherein the reagent or ions added by injectors can be the same or different, and the bio-physical or physical properties detected by different detectors can be the same or different.

45. (canceled)

46. (canceled)

47. The apparatus of claim 1, wherein the detector comprises one or more sensors placed partially or completely alongside one or more side walls of the channel or chamber; each sensor is independently a thermal sensor, optical sensor, acoustical sensor, biological sensor, chemical sensor, electro-mechanical sensor, electro-chemical sensor, electro-optical sensor, electro-thermal sensor, electro-chemical-mechanical sensor, bio-chemical sensor, bio-mechanical sensor, bio-optical sensor, electro-optical sensor, bio-electro-optical sensor, bio-thermal optical sensor, electro-chemical optical sensor, bio-thermal sensor, bio-physical sensor, bio-electro-mechanical sensor, bio-electro-chemical sensor, bio-electro-optical sensor, bio-electro-thermal sensor, bio-mechanical-optical sensor, bio-mechanical thermal sensor, bio-thermal-optical sensor, bio-electro-chemical-optical sensor, bio-electro-mechanical optical sensor, bio-electro-thermal-optical sensor, bio-electro-chemical-mechanical sensor, physical sensor, mechanical sensor, piezo-electrical sensor, piezo-electro phototronic sensor, piezo-phototronic sensor, piezo-electro optical sensor, bio-electrical sensor, bio-marker sensor, electrical sensor, magnetic sensor, electromagnetic sensor, image sensor, or radiation sensor; wherein the thermal sensor comprises a resistive temperature micro-sensor, a micro-thermocouple, a thermo-diode and thermo-transistor, or a surface acoustic wave (SAW) temperature sensor; the image sensor comprises a charge coupled device (CCD) or a CMOS image sensor (CIS); the radiation sensor comprises a photoconductive device, a photovoltaic device, a pyro-electrical device, or a micro-antenna; the mechanical sensor comprises a pressure micro-sensor, micro-accelerometer, flow meter, viscosity measurement tool, micro-pyrometer, or micro flow-sensor; the magnetic sensor comprises a magneto-galvanic micro-sensor, a magneto-resistive sensor, a magneto diode, or magneto-transistor; the biochemical sensor comprises a conductimetric device, a bio-marker, a bio-marker attached to a probe structure, or a potentiometric device.

48. (canceled)

49. (canceled)

50. The apparatus of claim 47, wherein at least one sensor is a probing sensor and applies a probing or disturbing signal to the biological subject; and the same sensor or at least another different sensor detects a response from the biological subject upon which the probing or disturbing signal is applied.

51. (canceled)

52. The apparatus of claim 50, further comprising a read-out circuitry which is connected to at least one sensor and transfers data from the sensor to a recording device; wherein the connection between the read-out circuit and the sensor is digital, analog, optical, thermal, piezo-electrical, piezo-photronic, piezo-electrical photronic, opto-electrical, electro-thermal, opto-thermal, electric, electromagnetic, electromechanical, or mechanical.

53. (canceled)

54. The apparatus of claim 1, wherein the chamber or channel has a length ranging from 1 micron to 50,000 microns, from 1 micron to 15,000 microns, from 1 micron to 10,000 microns, from 1.5 microns to 5,000 microns, or from 3 microns to 1,000 microns; or a width or height ranging from 0.1 micron to 100 microns; from 0.1 micron to 25 microns, from 1 micron to 15 microns, or from 1.2 microns to 10 microns.

55. (canceled)

56. (canceled)

57. The apparatus of claim 1, wherein the apparatus further comprises an application specific integrated circuit chip which is internally bonded to or integrated into the detector, or a waste collection system for collecting a waste liquid; or wherein the apparatus is fabricated by integrated circuit technologies.

58. (canceled)

59. (canceled)

60. (canceled)

61. (canceled)

62. (canceled)

63. The apparatus of claim 37, wherein the overall cancer risk assessment can be pre-cancer or early stage cancer detection information; the bio-markers comprise alpha-fetoprotein, prostate specific antigen, arcinoembryonic antigen, carbohydrate antigen 50, carbohydrate antigen 242, carbohydrate antigen 125, carbohydrate antigen 153, carbohydrate antigen 199, or carbohydrate antigen 724.

64-172. (canceled)
